SEQUENCE LISTING (33 sequences)

SEQ ID NO: 1 - DNA, 42 bp, synthetic construct
  source 1..42: mol_type = other DNA
ggacgacgccagaagtttacgaggatatggtaacatagtcgt

SEQ ID NO: 2 - protein, 8 aa, synthetic construct
EKEKEPPC

SEQ ID NO: 3 - protein, 6 aa, synthetic construct
EKEPPC

SEQ ID NO: 4 - protein, 7 aa, synthetic construct
EKEKEPC

SEQ ID NO: 5 - protein, 5 aa, synthetic construct
EKEPC

SEQ ID NO: 6 - protein, 4 aa, synthetic construct
EPPC

SEQ ID NO: 7 - protein, 9 aa, synthetic construct
EKEKEPPPC

SEQ ID NO: 8 - protein, 7 aa, synthetic construct
EKEPPPC

SEQ ID NO: 9 - protein, 5 aa, synthetic construct
EPPPC

SEQ ID NO: 10 - protein, 8 aa, synthetic construct
  SITE 8: note = the C-terminus is amidated
EKEKEPPC

SEQ ID NO: 11 - protein, 6 aa, synthetic construct
  SITE 6: note = the C-terminus is amidated
EKEPPC

SEQ ID NO: 12 - protein, 7 aa, synthetic construct
  SITE 7: note = the C-terminus is amidated
EKEKEPC

SEQ ID NO: 13 - protein, 5 aa, synthetic construct
  SITE 5: note = the C-terminus is amidated
EKEPC

SEQ ID NO: 14 - protein, 4 aa, synthetic construct
  SITE 4: note = the C-terminus is amidated
EPPC

SEQ ID NO: 15 - protein, 9 aa, synthetic construct
  SITE 9: note = the C-terminus is amidated
EKEKEPPPC

SEQ ID NO: 16 - protein, 7 aa, synthetic construct
  SITE 7: note = the C-terminus is amidated
EKEPPPC

SEQ ID NO: 17 - protein, 5 aa, synthetic construct
  SITE 5: note = the C-terminus is amidated
EPPPC

SEQ ID NO: 18 - protein, 8 aa, synthetic construct
CPPKEKEK

SEQ ID NO: 19 - protein, 6 aa, synthetic construct
CPPKEK

SEQ ID NO: 20 - protein, 7 aa, synthetic construct
CPKEKEK

SEQ ID NO: 21 - protein, 5 aa, synthetic construct
CPKEK

SEQ ID NO: 22 - protein, 4 aa, synthetic construct
CPPK

SEQ ID NO: 23 - protein, 9 aa, synthetic construct
CPPPKEKEK

SEQ ID NO: 24 - protein, 7 aa, synthetic construct
CPPPKEK

SEQ ID NO: 25 - protein, 5 aa, synthetic construct
CPPPK

SEQ ID NO: 26 - protein, 8 aa, synthetic construct
  SITE 1: note = the N-terminus is acetylated
CPPKEKEK

SEQ ID NO: 27 - protein, 6 aa, synthetic construct
  SITE 1: note = the N-terminus is acetylated
CPPKEK

SEQ ID NO: 28 - protein, 7 aa, synthetic construct
  SITE 1: note = the N-terminus is acetylated
CPKEKEK

SEQ ID NO: 29 - protein, 5 aa, synthetic construct
  SITE 1: note = the N-terminus is acetylated
CPKEK

SEQ ID NO: 30 - protein, 4 aa, synthetic construct
  SITE 1: note = the N-terminus is acetylated
CPPK

SEQ ID NO: 31 - protein, 9 aa, synthetic construct
  SITE 1: note = the N-terminus is acetylated
CPPPKEKEK

SEQ ID NO: 32 - protein, 7 aa, synthetic construct
  SITE 1: note = the N-terminus is acetylated
CPPPKEK

SEQ ID NO: 33 - protein, 5 aa, synthetic construct
  SITE 1: note = the N-terminus is acetylated
CPPPK

(19) United States
(12) Patent Application Publication
(10) Pub. No.: US 2026/0013755 A1
(43) Pub. Date: Jan. 15, 2026

(54) APTAMER-BASED ANALYTE MONITORING SYSTEM

(71) Applicant: Biolinq Incorporated, San Diego, CA (US)

(72) Inventors: Jonathan HARRIS, San Diego, CA (US); Jonathan Everett KAVNER, San Diego, CA (US); Kyle Reed MALLIRES, San Diego, CA (US); Bo WANG, San Diego, CA (US)

(21) Appl. No.: 18/403,694

(22) Filed: Jan. 3, 2024

Related U.S. Application Data
(60) Provisional application No. 63/478,482, filed on Jan. 4, 2023.
(60) Provisional application No. 63/506,035, filed on Jun. 2, 2023.

(51) Int. Cl. A61B 5/1473; A61B 5/00

(52) U.S. Cl. CPC A61B 5/1473; A61B 5/685; A61B 2562/12

(57) ABSTRACT

Described herein are variations of a sensor configured to generate a signal that is indicative of a concentration of an analyte in a fluid. The sensor may include a working electrode comprising an electrode material, a biorecognition layer disposed at least partially on the electrode material and comprising a biorecognition element that selectively and reversibly binds to an analyte, and an at least partially desiccated hydrogel disposed on the biorecognition layer. In some variations, the biorecognition element may be functionalized with a redox-active molecule and is configured to change conformation upon binding the analyte to move the redox-active molecule, closer to, or further from, the electrode material. In some variations, the biorecognition element may be an aptamer.

### CROSS-REFERENCE TO RELATED APPLICATIONS

This application claims the benefit of U.S. Provisional Patent Application No. 63/478,482, filed Jan. 4, 2023, and U.S. Provisional Patent Application No. 63/506,035, filed Jun. 2, 2023, the content of which is herein incorporated by reference in its entirety.

### STATEMENT REGARDING FEDERAL SPONSORED RESEARCH

This Invention was made with U.S. Government support pursuant to a grant by Air Force Research Laboratory under agreement number FA8650-18-2-5402. The U.S. Government has certain rights in the Invention.

### REFERENCE TO AN ELECTRONIC SEQUENCE LISTING

The contents of the electronic sequence listing submitted electronically herewith (filename: BLNQ_003_03US_SeqList_ST26.xml; Size: 35,571 bytes; and Date of Creation: Jan. 2, 2024) are hereby incorporated by reference in their entirety.

### BACKGROUND

Nucleic acid-based electrochemical sensors are a versatile technology enabling affinity-based detection of a great variety of molecular targets, regardless of inherent electrochemical activity or enzymatic reactivity. Additionally, their modular interface and ease of fabrication enable rapid prototyping and sensor development. However, the technology has inhibiting limitations in terms of long-term stability that have complicated translation into clinically valuable platforms like continuous molecular monitors. For instance, because a biorecognition surface of a sensor for biomolecules typically has a relatively complex molecular structure with one or more macromolecules arranged in an ordered fashion, with the one or more macromolecules acting as a biorecognition element capable of selectively binding or otherwise interacting with a desired analyte, a biorecognition surface tends to be relatively delicate and susceptible to degradation and damage caused by, for example, mechanical perturbance, desiccation, radiation exposure, etc. There is, therefore, a need for methods and materials that can improve sensor stability while preserving sensor sensitivity.

### SUMMARY

Described herein are devices, systems, and methods related to a sensor configured to generate a signal that is indicative of a concentration of an analyte in a fluid.

According to an embodiment, the present disclosure further relates to a working electrode, comprising: an electrode material; a biorecognition element disposed on the electrode material and configured to selectively and reversibly bind to an analyte in a fluid; a first thiol-based passivation molecule disposed on the electrode material; and a second, different thiol-based passivation molecule disposed on the electrode material. In some embodiments, the first thiol-based passivation molecule is 6-mercapto-1-hexanol and the second, different thiol-based passivation molecule is 2-((3-((6-mercaptohexyl)thio)-2-methylpropanoyl)oxy)ethyl (2-trimethylammonium)ethyl)phosphate. In some embodiments, a distal end of the first thiol-based passivation molecule, the second, different thiol-based passivation molecule, or both, comprises one or more of a hydrophilic moiety, a hydrophobic moiety, a charged moiety, and a zwitterionic moiety. In some embodiments, the distal end of the first thiol-based passivation molecule, the second, different thiol-based passivation molecule, or both, comprises a zwitterionic moiety and the zwitterionic moiety is a zwitterionic phosphorylcholine head group. In some embodiments, the first thiol-based passivation molecule, the second, different thiol-based passivation molecule, or both, is a zwitterionic peptide. In some embodiments, the biorecognition element is an aptamer. In some embodiments, the sensor further comprises a microelectrode array, and the working electrode is part of the microelectrode array.

According to an embodiment, the present disclosure further relates to a method of manufacturing a working electrode for an analyte sensor configured to generate a signal indicative of a concentration of an analyte in a fluid, the method comprising: providing a working electrode comprising an electrode material and a biorecognition element deposited on the electrode material, wherein the biorecognition element is configured to selectively and reversibly bind to the analyte; applying, for a first predetermined time period, a first thiol-based passivation molecule to the electrode material; and applying, for a second predetermined time period, a second, different thiol-based passivation molecule to the electrode material. In some embodiments, the first thiol-based passivation molecule is 6-mercapto-1-hexanol (MCH) and the second, different thiol-based passivation molecule is 2-((3-((6-mercaptohexyl)thio)-2-methylpropanoyl)oxy)ethyl (2-trimethylammonium)ethyl)phosphate (PC). In some embodiments, applying the first thiol-based passivation molecule and the second thiol-based passivation molecule comprises contacting the electrode material with a solution comprising the first thiol-based passivation molecule and the second, different thiol-based passivation molecule at a ratio of MCH to PC of between about 1 mM and about 30 mM MCH to between about 2.5 mM and about 30 mM PC.

According to an embodiment, the present disclosure further relates to a wearable device comprising: an electrochemical, aptamer-based sensor comprising a first thiol-based passivation molecule and a second, different thiol-based passivation molecule on the electrode material, the first thiol-based passivation molecule comprising 6-mercapto-1-hexanol (MCH) and the second, different thiol-based passivation molecule comprising 2-((3-((6-mercaptohexyl)thio)-2-methylpropanoyl)oxy)ethyl (2-trimethylammonium)ethyl)phosphate (PC).

According to an embodiment, the present disclosure further relates to a sensor configured to generate a signal that is indicative of a concentration of an analyte in a fluid, the sensor comprising a working electrode comprising: an electrode material; a biorecognition layer disposed at least partially on the electrode material and comprising a biorecognition element that selectively and reversibly binds to an analyte; and an at least partially desiccated hydrogel disposed on the biorecognition layer. In some embodiments, the hydrogel comprises about 10%, about 20%, about 30%, about 40%, about 50%, about 60%, about 70%, about 80%, or about 90% of water compared to a fully hydrated state of the hydrogel. In some embodiments, the hydrogel is fully desiccated. In some embodiments, the sensor further comprises a microelectrode array, and the working electrode is part of the microelectrode array. In some embodiments, the biorecognition element is an aptamer.

According to an embodiment, the present disclosure further relates to a method of manufacturing a sensor configured to generate a signal that is indicative of a concentration of an analyte in a fluid, the method comprising: (a) providing a working electrode comprising an electrode material and a biorecognition layer disposed at least partially on the electrode material, wherein the biorecognition layer comprises a biorecognition element that selectively and reversibly binds to an analyte; (b) applying a hydrogel on the biorecognition layer; and (c) drying the hydrogel to an at least partially desiccated state. In some embodiments, the hydrogel comprises about 10%, about 20%, about 30%, about 40%, about 50%, about 60%, about 70%, about 80%, or about 90% of water compared to a fully hydrated state of the hydrogel. In some embodiments, the hydrogel is fully desiccated. In some embodiments, the sensor further comprises a microelectrode array, and the working electrode is part of the microelectrode array. In some embodiments, the biorecognition element is an aptamer.

According to an embodiment, the present disclosure further relates to a sensor comprising: a working electrode comprising an electrode material, a biorecognition layer disposed at least partially on the electrode material and comprising a biorecognition element that selectively and reversibly binds to an analyte, and a hydrogel disposed on the biorecognition layer, wherein the sensor is sterilized with exposure to a radiation, and the sterilized sensor is configured to generate a signal that is indicative of a concentration of an analyte in a fluid.

In some embodiments, the radiation is an ultraviolet radiation, a gamma radiation, an X-ray radiation, or an electron beam radiation. In some embodiments, the radiation is an electron beam radiation. In some embodiments, the biorecognition layer is degraded by the radiation exposure by not more than about 2%, not more than about 5%, not more than about 10%, not more than about 15%, or not more than about 20%. In some embodiments, the sensor further comprises a microelectrode array, and the working electrode is part of the microelectrode array. In some embodiments, the biorecognition element is an aptamer.

According to an embodiment, the present disclosure further relates to a method of sterilizing a sensor configured to generate a signal that is indicative of a concentration of an analyte in a fluid, the method comprising: (a) providing a working electrode comprising an electrode material and a biorecognition layer comprising a biorecognition element that selectively and reversibly binds to an analyte, wherein the biorecognition layer is disposed at least partially on the electrode material; (b) applying a hydrogel on the biorecognition layer; and (c) sterilizing the working electrode with exposure to a radiation. In some embodiments, the radiation is an ultraviolet radiation, a gamma radiation, an X-ray radiation, or an electron beam radiation. In some embodiments, the radiation is an electron beam radiation. In some embodiments, the biorecognition layer is degraded by the radiation exposure by not more than about 2%, not more than about 5%, not more than about 10%, not more than about 15%, or not more than about 20%. In some embodiments, the biorecognition element is an aptamer.

According to an embodiment, the present disclosure further relates to a sensor configured to generate a signal that is indicative of a concentration of an analyte in a fluid, the sensor comprising a working electrode comprising: an electrode material; a biorecognition layer disposed at least partially on the electrode material and comprising a biorecognition element that selectively and reversibly binds to an analyte; and a hydrogel disposed on the biorecognition layer, wherein the biorecognition layer degrades at a rate of not more than about 3% per day when stored at an ambient temperature of between about 15 degrees Celsius and about 30 degrees Celsius, and an ambient humidity of between about 10% and about 80% relative humidity. In some embodiments, the hydrogel comprises about 10%, about 20%, about 30%, about 40%, about 50%, about 60%, about 70%, about 80%, or about 90% of water compared to a fully hydrated state of the hydrogel. In some embodiments, the hydrogel is fully desiccated. In some embodiments, the sensor further comprises a microelectrode array, and the working electrode is part of the microelectrode array. In some embodiments, the biorecognition element is an aptamer.

According to an embodiment, the present disclosure further relates to a working electrode configured to generate a signal indicative of a concentration of an analyte in a fluid, the working electrode comprising: an electrode material; a biorecognition element disposed on the electrode material that selectively binds to the analyte, wherein the biorecognition element is functionalized with a redox-active molecule and is configured to change conformation upon binding the analyte to move the redox-active molecule, closer to, or further from, the electrode material; and a passivation element comprising a zwitterionic peptide disposed on the electrode material. In some embodiments, the C-terminus of the C-terminal cysteine is a free carboxyl group or a modified C-terminus having a neutral charge. In some embodiments, the N-terminus of the N-terminal cysteine is a free amine group or modified N-terminus having a neutral charge. In some embodiments, the zwitterionic peptide consists of a peptide sequence of X-(KX)x-PyC or CPy-(KX)x-K, wherein x is between 1 and 5, X is a glutamic acid residue or an aspartic acid residue, K is a lysine residue, P is a proline residue, and C is a cysteine residue. In some embodiments, the biorecognition element is an aptamer.

According to an embodiment, the present disclosure further relates to a working electrode configured to generate a signal indicative of a concentration of an analyte in a fluid, the working electrode comprising: an electrode material; a biorecognition element disposed on the electrode material that selectively binds to the analyte; and a passivation element comprising a zwitterionic peptide disposed on the electrode material that comprises an amino acid sequence of X-(KX)x-PyC or CPy-(KX)x-K, wherein x is 0, 1 or 2, X is a glutamic acid residue or an aspartic acid residue, K is a lysine residue, P is a proline residue, and C is a cysteine residue.

In some embodiments, the amino acid sequence of the zwitterionic peptide is X-(KX)x-PyC comprising a C-terminal cysteine, and the C-terminus of the C-terminal cysteine is a free carboxyl group or a modified C-terminus having a neutral charge. In some embodiments, the amino acid sequence of the zwitterionic peptide is CPy-(KX)x-K comprising an N-terminal cysteine, and the N-terminus of the N-terminal cysteine residue is a free amine group or a modified N-terminus having a neutral charge. In some embodiment, the zwitterion peptide consists of the amino acid sequence of any one of SEQ ID NO: 2-33. In some embodiments, the biorecognition element is an aptamer.

According to an embodiment, the present disclosure further relates to a zwitterionic peptide comprising an amino acid sequence of X-(KX)x-PyC or CPy-(KX)x-K, wherein x is 0, 1 or 2, X is a glutamic acid residue or an aspartic acid residue, K is a lysine residue, P is a proline residue, and C is a cysteine residue. In some embodiments, the amino acid sequence of the zwitterionic peptide is X-(KX)x-PyC comprising a C-terminal cysteine, and the C-terminus of the C-terminal cysteine is a free carboxyl group or a modified C-terminus having a neutral charge. In some embodiments, the amino acid sequence of the zwitterionic peptide is CPy-(KX)x-K comprising an N-terminal cysteine, and the N-terminus of the N-terminal cysteine residue is a free amine group or a modified N-terminus having a neutral charge. In some embodiment, the zwitterion peptide consists of the amino acid sequence of any one of SEQ ID NO: 2-33. In some embodiments, the biorecognition element is an aptamer.

According to an embodiment, the present disclosure further relates to a method for manufacturing a working electrode, comprising: depositing gold on a substrate to create a gold surface; depositing a biorecognition element on the gold surface, wherein the biorecognition element is functionalized with a redox-active molecule and is configured to change conformation upon binding the analyte to move the redox-active molecule, closer to, or further from, the gold surface; and depositing a passivation element comprising a zwitterionic peptide on the gold surface.

Both the foregoing general description and the following detailed description are exemplary and explanatory only and are not restrictive. Further features and/or variations may be provided in addition to those set forth herein. For example, the implementations described herein may be directed to various combinations and sub-combinations of the disclosed features.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an illustrative schematic of an analyte monitoring system with a microneedle array.

FIG. 2A depicts an illustrative schematic of an analyte monitoring device.

FIG. 2B depicts an illustrative schematic of microneedle insertion depth in an analyte monitoring device.

FIGS. 3A-3D depict an upper perspective view, a side view, a bottom view, and an exploded view, respectively, of an analyte monitoring device.

FIGS. 4A-4E depict a perspective exploded view, a side exploded view, a lower perspective view, a side view, and an upper perspective view, respectively, of a sensor assembly in an analyte monitoring device.

FIGS. 4F-4H depict a perspective exploded view, a side exploded view, and a side view, respectively, of a sensor assembly in an analyte monitoring device.

FIG. 5A depicts an illustrative schematic of a microneedle array. FIG. 5B depicts an illustrative schematic of a microneedle in the microneedle array depicted in FIG. 5A.

FIG. 6 depicts an illustrative schematic of a microneedle array used for sensing multiple analytes, of which at least one of the multiple analytes is cortisol.

FIG. 7A depicts a cross-sectional side view of a columnar microneedle having a tapered distal end. FIGS. 7B and 7C are images depicting perspective and detailed views, respectively, of an embodiment of the microneedle shown in FIG. 7A.

FIG. 8 depicts an illustrative schematic of a columnar microneedle having a tapered distal end.

FIG. 9 depicts a cross-sectional side view of a columnar microneedle having a tapered distal end.

FIGS. 10A and 10B depict illustrative schematics of a microneedle array and a microneedle, respectively. FIGS. 10C-10F depict detailed partial views of an illustrative variation of a microneedle.

FIGS. 11A and 11B depict an illustrative variation of a microneedle.

FIGS. 12A and 12B depict illustrative schematics of a microneedle array configuration.

FIGS. 12C and 12D depict illustrative schematics of a microneedle array configuration.

FIGS. 13A and 13B depict perspective and orthogonal views, respectively, of an illustrative variation of a die including a microneedle array.

FIGS. 14A-14J depict illustrative schematics of different variations of microneedle array configurations.

FIG. 15 depicts illustrative schematics of a planar microelectrode array.

FIGS. 16A-16C depict illustrative schematics of layered structures of a working electrode, a counter electrode, and a reference electrode, respectively.

FIGS. 16D-16F depict illustrative schematics of layered structures of a working electrode, a counter electrode, and a reference electrode, respectively.

FIGS. 16G-16I depict illustrative schematics of layered structures of a working electrode, a counter electrode, and a reference electrode, respectively.

FIG. 16J depict illustrative schematics of layered structures of a working electrode in which the biorecognition element is an analyte-binding aptamer.

FIG. 16K depicts an illustrative schematic of a layered structure of a working electrode in which the biorecognition element is an analyte-binding aptamer, as shown in FIG. 16J, with a biocompatible layer applied onto the biorecognition layer.

FIG. 16L depicts an illustrative schematic of a layered structure of a working electrode in which the biorecognition element is an analyte-binding aptamer, as shown in FIG. 16J, wherein the biorecognition layer comprises a multi-component passivation element.

FIG. 16M depicts an illustrative schematic of a layered structure of a working electrode in which the biorecognition element is an analyte-binding aptamer, as shown in FIG. 16J, and the biorecognition layer comprises a multi-component passivation element.

FIGS. 17A-17K depict illustrative schematics of layered structures of a working electrode in which the biorecognition element is an analyte-binding aptamer.

FIGS. 18A-18B are a flow diagram of a method of applying a passivation element to a working electrode as part of a biorecognition layer.

FIGS. 19A and 19B depict illustrative schematics of a housing of an analyte monitoring device including a user interface with indicator light elements.

FIGS. 20A-20C depict illustrative schematics of illumination modes in an analyte monitoring device for indicating analyte measurement data.

FIGS. 21A-21B depict illustrative schematics of an exemplary working electrode functionalized with cortisol-binding aptamers before (FIG. 21A) and after (FIG. 21B) application of a hydrogel biocompatible layer.

FIG. 21C depict illustrative schematics of an exemplary microelectrode array comprising the working electrode shown in FIG. 21B.

FIG. 22A depicts a plot showing cortisol detection with a hydrogel-covered working electrode comprising cortisol-binding aptamers before and after overnight drying.

FIG. 22B depicts a plot showing cortisol detection with a bare (non-hydrogel-covered) working electrode comprising cortisol-binding aptamers before and after overnight drying.

FIG. 23A depicts a plot showing cortisol detection with a hydrogel-covered working electrode comprising cortisol-binding aptamers before and after 2.5 days of drying, wherein the electrode is encapsulated with a hydrogel biocompatible layer.

FIG. 23B depicts a plot showing cortisol detection with a bare (non-hydrogel-covered) working electrode comprising cortisol-binding aptamers before and after 2.5 days of drying.

FIG. 24A depicts a plot showing cortisol detection with a hydrogel-covered working electrode comprising cortisol-binding aptamers before and after 2.5 days of drying.

FIG. 24B depicts a plot showing cortisol detection with a bare (non-hydrogel-covered) working electrode comprising cortisol-binding aptamers before and after 2.5 days of drying.

FIG. 24C depicts a plot showing initial cortisol detection with a bare or a hydrogel-covered working electrodes comprising cortisol-binding aptamers shortly after production.

FIG. 24D depicts a plot showing cortisol detection with a bare or a hydrogel-covered working electrodes comprising cortisol-binding aptamers after 2.5 days of drying.

FIG. 25A depicts a plot showing cortisol detection with a hydrogel-covered working electrode comprising cortisol-binding aptamers before and after electron beam sterilization.

FIG. 25B depicts a plot showing cortisol detection with a bare (non-hydrogel-covered) working electrode comprising cortisol-binding aptamers before and after electron beam sterilization.

FIG. 25C depicts a plot showing cortisol detection with a bare or a hydrogel-covered working electrodes comprising cortisol-binding aptamers after electron beam sterilization.

FIG. 26 depicts an exemplary zwitterionic peptide for use in the sensors described herein.

FIG. 27 depicts a plot showing cortisol detection in a control sensor using 6-merkaptohexanol as a passivation element, and an experimental cortisol sensor using the zwitterionic peptide shown in FIG. 26 as the passivation element.

FIGS. 28-30 depict plots of E-AB sensor stability over time when varying multi-component passivation elements are used.

FIGS. 31 and 33 depict plots of peak current measured for E-AB sensors at different frequencies, each of the E-AB sensors having different multi-component passivation elements.

FIGS. 32 and 34 depict plots of gain measured for E-AB sensors at different frequencies, each of the E-AB sensors having different multi-component passivation elements.

FIG. 35 depicts a plot of noise level for E-AB sensors at different frequencies, each of the E-AB sensors having different multi-component passivation elements.

FIG. 36 depicts a scanning electron micrograph of an exemplary biocompatible layer deposited onto a microneedle array.

### DETAILED DESCRIPTION

Non-limiting examples of various aspects and variations of the invention are described herein and illustrated in the accompanying drawings.

As used herein, the term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or for the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

An aptamer is a single-stranded oligonucleotide or a peptide that folds into a defined structure that selectively binds to a specific analyte (which may be referred to as target), which may be, by way of example, a protein, a peptide, a hormone, a nucleic acid, or a small molecule. Aptamers with affinity for a desired target may be conventionally selected from a large oligonucleotide library through a process called SELEX (Systematic Evolution of Ligands by Exponential Enrichment). Through an iterative process, non-binding aptamers are discarded and aptamers binding to the proposed target are amplified by polymerase chain reaction (PCR). The iterative process may include counter-selection (using interferents and structurally similar molecules) to discard aptamers with insufficient selectivity toward analytes. Moreover, the conformational change of the aptamer effected by target binding and dissociation can be used to effect electrical, electrochemical, or chemical changes that can be harnessed to visualize the target binding/dissociation through an assay or sensor. If needed, the selected aptamers can be further modified (e.g., introduce truncations and mutations) to improve the aptamer conformational changes, thereby improving sensor signals. These properties make aptamers an attractive “biorecognition” element for use in detecting one or more desired analytes.

Since the development of the continuous glucose monitor, electrochemical biosensors have gained widespread attention for their potential value in biomedical applications. While the glucose monitor achieves selectivity from a surface-immobilized enzyme, glucose oxidase, another class of electrochemical biosensors uses nucleic acids in place of enzymes. These nucleic acid-based electrochemical sensors (NBEs) rely on affinity of surface-bound oligonucleotides for specific target molecules. Upon target binding, signal is generated through a change in electron transfer kinetics of either a covalently-attached or solvated redox reporter. By way of example, in an NBE such as an electrochemical aptamer-based sensor, the surface of a working electrode may be functionalized with an aptamer (e.g., an analyte-binding aptamer) configured to selectively and reversibly bind a given analyte. Moreover, the aptamer is modified with the addition of a redox-active molecule. The aptamer may be configured so that, upon binding the analyte, the analyte-binding aptamer experiences a conformational change that moves the redox-active molecule closer, or further, from the electrode. The movement of the redox-active molecule may be detected as an analyte concentration-dependent electrochemical signal. Because NBEs rely on affinity instead of target reactivity, they can be developed for many molecules of interest: from a complementary nucleic acid to small molecule drugs, protein biomarkers, and even whole viruses or cells. However, NBEs have limitations preventing their translation into clinically valuable platforms, such as wearable analyte monitors.

One such limitation of NBEs is their lack of compatibility with long term storage. NBEs degrade quickly when stored dry. When the NBEs are dried, there may be damage to the sensing structure itself and/or to the chemistry linking the sensing chemistry to the electrode. Hydrating the sensing surface may improve the ability to preserve the sensing chemistry, but this is not feasible for a variety of reasons, including e.g., packing and shipping.

Accordingly, described herein is a biocompatible layer comprising a hydrogel, which can be added to an NBE platform and provide a means to preserve a biorecognition element (i.e., sensing chemistry) even when dried. The hydrogel biocompatible layer may allow the NBE platform to be safely and stably shipped, packed, and/or stored. When compared with a control sensor without a hydrogel biocompatible layer, sensors comprising the hydrogel biocompatible layer described herein may achieve zero loss in sensor signal when dried overnight. Further, the hydrogel biocompatible layer may also protect the biorecognition element from e-beam sterilization.

Another such limitation of NBEs is their lack of stability over time. For instance, while NBEs are relatively stable in blood and serum in vitro, they exhibit significant drift when deployed directly in the living body. This issue presumably arises due at least in part to degradation of the target-recognizing sensing chemistry and the non-specific adsorption of cells and other components to the sensor surface. Non-specific adsorption, which may be referred to as fouling, may increase undesirable background current. When sensors are exposed to blood or serum, proteins from those fluids deposit on the monolayer surface. These proteins, which form a fouling layer, simultaneously restrict the conformational dynamics of electrode attached nucleic acids and reduce electron transfer efficiency between the electrode and redox reporter. Fouling-induced hindering of conformational dynamics reduces the total signal gain possible from target binding, while worsening electron transfer reduces overall signal output, regardless of target concentration. Taken together, these effects limit the signaling accuracy and signaling lifetime of NBEs.

To address these concerns, mathematical corrections and/or sensor surface modifications have been used. As an example, surface modifications have included approaches to passivate the sensor surface. In the case of sensors comprising gold electrodes, this includes leveraging the chemistry of thiol on gold self-assembly to form monolayers containing short-chain alkylthiols for electrode surface passivation. With particular reference to NBE sensors, and further to the above, the typical E-AB sensor architecture comprises a sensing chemistry on a portion of an electrode surface with the remainder of the electrode surface being covered with a small molecule blocking group also attached with a thiol. In this way the small molecule blocking group serves as a passivation element on the sensor surface. To date, only a single type of small molecule thiol has been explored. Such approach, however, is often inadequate, as a passivation element must simultaneously achieve multiple, sometimes competing goals.

For instance, the passivation element must have enough intermolecular interaction with itself (meaning molecules of the same type) that it forms a densely packed layer on the gold surface, thereby providing good passivation. Good passivation is key for good resolution of the MB peak (which leads to more accurate measurements) and long-term stability. At the same time, the passivation element must not passivate so well that it blocks electron transfer from the redox probe. The longer each passivation molecule is (e.g., 6 carbons vs 10 carbons vs 16 carbons), the stronger its intermolecular attraction and the more densely it will pack, leading to better passivation and long-term stability but to less efficient electron transport from the redox probe. This is because the electron has to tunnel through the passivation element to reach the electrode surface and, as the thickness of the passivation element increases, electron transport from the redox reporter becomes less probable. Measured currents, accordingly, decrease across a homologous series. Thus, thick layers will have only a tiny signal from the MB (or no signal at all), leading to low signal-to-noise ratio (SNR). Conversely, thin layers will have a strong signal from MB but poor passivation and poor stability.

In another instance, the passivation element needs to be compatible with the aptamer and the redox probe so as not to interfere and prevent binding with the target analyte or prevent structural switching of the aptamer. For example, the use of a passivation element terminating in a hydrophobic moiety may reduce signal transduction, as the aptamer and/or redox probe may interact with the hydrophobic passivation element, ‘sticking’ to its surface or becoming partially embedded therein. Either interaction would prevent the structural switching mechanism (analogous to irreversible denaturing seen in proteins). The use of a passivation element terminating in a hydrophilic moiety (i.e., a polar or a charged moiety), which may be a polar hydroxyl group, a charged sulfonate, a carboxylate, a multiply charged group, or a zwitterionic group (i.e., equal number positive and negative charges), can result in a functional “blocking” layer. However, a hydrophilic head group may allow for more ingress of aqueous electrolyte into the passivation element. Thus, if the passivation element is a relatively thin (e.g., 6 carbons) thiol layer, then it may become prone to degradation.

In view of the above, the challenge of balancing thickness, packing density, and hydrophilicity of the passivation element can be readily appreciated. An ideal passivation element would provide additional benefits, such as preventing biofouling and providing points of covalent attachment (e.g., vinyl, epoxide, acrylate, methacrylate, benzophenone, azide) for a subsequent biocompatible protective layer, thereby improving the overall mechanical integrity of the analyte sensor. However, there is a limit to what can be accomplished with a single small molecule thiol. In fact, it is unlikely a single molecule can satisfy each of the requirements noted above. Moreover, there is a limit to the types of thiol molecules that are commercially available or that can easily be synthesized by a chemist. Many theoretical thiols may exist that could serve as blocking groups, but there are technical and practical barriers to discovering and utilizing them. Accordingly, described herein are a multi-component passivation elements that simultaneously provide improvements in thickness, packing density, and hydrophilicity. Generally, a multi-component passivation element may comprise a thiol-mixture comprising two or more thiols that form the multi-component passivation element, each thiol providing its own benefits while minimizing the other thiol's disadvantages. In this manner, the E-AB sensors described herein may exhibit improved fidelity, improved long-term stability, improved biocompatibility, reduced biofouling, and/or improved structural integrity.

Specifically, the multi-component passivation element described herein improves stability by suppressing oxygen reduction reactions at the electrode surface (thereby reducing sensor degradation), suppressing electrode capacitance, reducing variance in sensor response to target molecules (e.g., improving manufacturing batch yield and/or decreasing sensor degradation), and/or reducing variance in the limit of detection to target molecules. Moreover, the multi-component passivation element reduces sensor degradation by increasing the baseline current and changing the redox probe peak current value when measured against baseline.

Additionally or alternatively to the above-described multi-component passivation element, passivation elements described herein may include zwitterions. A zwitterion may in some cases be a peptide, and in those cases may be referred to as a “zwitterionic peptide”. As described herein, using a zwitterionic peptide as a component in the passivation element in a sensor as described herein may provide several advantages, including, but not limited to: (1) improved hydrophilicity of the electrode surface; (2) better blocking of the non-specific adsorption of proteins on the electrode surface in vivo; (3) reduced need of biologically incompatible reagents in the production process, (4) non-odorous, thereby removing the need for a fume hood during the production process; and (5) lower toxicity of an electrode functionalized therewith, when applied to or inserted within a subject.

In some embodiments, the multi-component passivation element described herein may comprise both small molecule thiols and zwitterions, such as, for example, a mixture of small molecule thiols and zwitterions. The small molecule thiols may include one or more different small molecule thiols described herein. Similarly, the zwitterions may include one or more different zwitterions described herein. Accordingly, the multi-component passivation element may include one or more small molecule thiol and one or more zwitterions, in any combination as described in more detail herein.

The following are analyte monitoring systems that may utilize hydrogel biocompatible layers and multi-component passivation elements. In some variations, the hydrogel additions and passivation elements may be utilized with analyte monitoring systems that may, include microneedle arrays. The following descriptions are meant to be exemplary, and aspects related to the aptamer-based approach for measuring and monitoring analyte consistent with the current subject matter are not limited to the example analyte monitoring device and the example microneedle arrays described herein.

### Analyte Monitoring System

As generally described herein, an analyte monitoring system may include an analyte monitoring device that is worn by a user and includes one or more sensors for monitoring an analyte in a user. The sensors may, for example, include one or more electrodes configured to perform electrochemical detection of the analyte. The analyte monitoring device may communicate sensor data to an external computing device for storage, display, and/or analysis of sensor data.

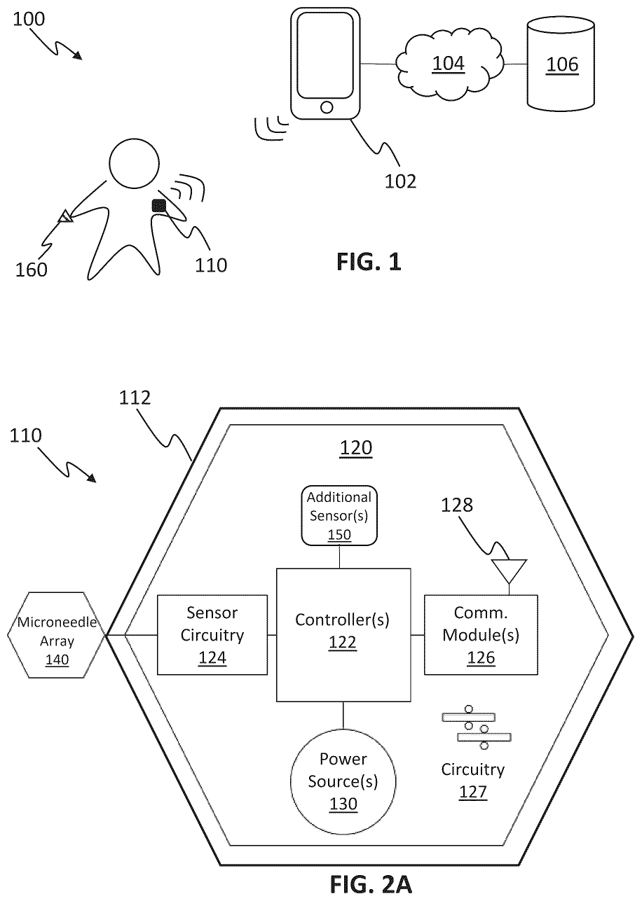

For example, as shown in FIG. 1, an analyte monitoring system 100 may include an analyte monitoring device 110 that is worn by a user, and the analyte monitoring device 110 may be a continuous analyte monitoring device. The analyte monitoring device 110 may include, for example, a microneedle array comprising at least one electrochemical sensor for detecting and/or measuring analyte in body fluid of a user. In some variations, the analyte monitoring device may be applied to the user using suitable applicator 160 or may be applied manually. The analyte monitoring device 110 may include one or more processors for performing analysis on sensor data, and/or a communication module (e.g., wireless communication module) configured to communicate sensor data to a mobile computing device 102 (e.g., smartphone) or another suitable computing device. In some variations, the mobile computing device 102 may include one or more processors executing a mobile application to handle sensor data (e.g., displaying data, analyzing data for trends, etc.) and/or provide suitable alerts or other notifications related to the sensor data and/or analysis thereof. It should be understood that while in some variations the mobile computing device 102 may perform sensor data analysis locally, other computing device(s) may alternatively or additionally remotely analyze sensor data and/or communicate information related to such analysis with the mobile computing device 102 (or other suitable user interface) for display to the user. Furthermore, in some variations the mobile computing device 102 may be configured to communicate sensor data and/or analysis of the sensor data over a network 104 to one or more storage devices 106 (e.g., server) for archiving data and/or other suitable information related to the user of the analyte monitoring device.

The analyte monitoring devices described herein have characteristics that improve a number of properties that are advantageous for a continuous analyte monitoring device. For example, the analyte monitoring devices described herein have reduced degradation of and/or damage to the sensor over time, and improved stability to help minimize change in sensor response through storage and operation of the analyte monitoring device.

Various aspects of example variations of the analyte monitoring systems, and methods of use thereof, are described in further detail below.

### Analyte Monitoring Device

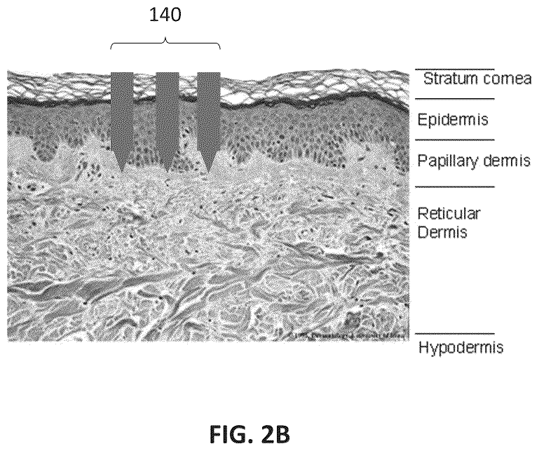

As shown in FIG. 2A, in some variations, an analyte monitoring device 110 may generally include a housing 112 and a microneedle array 140 extending outwardly from the housing. The housing 112, may, for example, be a wearable housing configured to be worn on the skin of a user such that the microneedle array 140 extends at least partially into the skin of the user. For example, the housing 112 may include an adhesive such that the analyte monitoring device 110 is a skin-adhered patch that is simple and straightforward for application to a user. The microneedle array 140 may be configured to puncture the skin of the user and include one or more electrochemical sensors (e.g., electrodes) configured for measuring analyte that is accessible after the microneedle array 140 punctures the skin of the user. In some variations, the analyte monitoring device 110 may be integrated or self-contained as a single unit, and the unit may be disposable (e.g., used for a period of time and replaced with another instance of the analyte monitoring device 110).

An electronics system 120 may be at least partially arranged in the housing 112 and include various electronic components, such as sensor circuitry 124 configured to perform signal processing (e.g., biasing and readout of electrochemical sensors, converting the analog signals from the electrochemical sensors to digital signals, etc.). The electronics system 120 may also include at least one microcontroller 122 for controlling the analyte monitoring device 110, at least one communication module 126, at least one power source 130, and/or other various suitable passive circuitry 127. The microcontroller 122 may, for example, be configured to interpret digital signals output from the sensor circuitry 124 (e.g., by executing a programmed routine in firmware), perform various suitable algorithms or mathematical transformations (e.g., calibration, etc.), and/or route processed data to and/or from the communication module 126. In some variations, the communication module 126 may include a suitable wireless transceiver (e.g., Bluetooth transceiver or the like) for communicating data with an external computing device 102 via one or more antennas 128. For example, the communication module 126 may be configured to provide uni-directional and/or bi-directional communication of data with an external computing device 102 that is paired with the analyte monitoring device 110. The power source 130 may provide power for the analyte monitoring device 110, such as for the electronics system. The power source 130 may include battery or other suitable source, and may, in some variations, be rechargeable and/or replaceable. Passive circuitry 127 may include various non-powered electrical circuitry (e.g., resistors, capacitors, inductors, etc.) providing interconnections between other electronic components, etc. The passive circuitry 127 may be configured to perform noise reduction, biasing and/or other purposes, for example. In some variations, the electronic components in the electronics system 120 may be arranged on one or more printed circuit boards (PCB), which may be rigid, semi-rigid, or flexible, for example. Additional details of the electronics system 120 are described further below.

In some variations, the analyte monitoring device 110 may further include one or more additional sensors 150 to provide additional information that may be relevant for user monitoring. For example, the analyte monitoring device 110 may further include at least one temperature sensor (e.g., thermistor) configured to measure skin temperature, thereby enabling temperature compensation for the sensor measurements obtained by the microneedle array electrochemical sensors.

In some variations, the microneedle array 140 in the analyte monitoring device 110 may be configured to puncture skin of a user. As shown in FIG. 2B, when the device 110 is worn by the user, the microneedle array 140 may extend into the skin of the user such that electrodes on distal regions of the microneedles rest in the dermis. Specifically, in some variations, the microneedles may be designed to penetrate the skin and access the upper dermal region (e.g., papillary dermis and upper reticular dermis layers) of the skin, in order to enable the electrodes to access interstitial fluid that surrounds the cells in these layers. For example, in some variations, the microneedles may have a height generally ranging between at least 350 μm and about 515 μm. In some variations, one or more microneedles may extend from the housing such that a distal end of the electrode on the microneedle is located less than about 5 mm from a skin-interfacing surface of the housing, less than about 4 mm from the housing, less than about 3 mm from the housing, less than about 2 mm from the housing, or less than about 1 mm from the housing.

In contrast to traditional continuous monitoring devices, which include sensors typically implanted between about 8 mm and about 10 mm beneath the skin surface in the subcutis or adipose layer of the skin, the analyte monitoring device 110 has a shallower microneedle insertion depth of about 0.25 mm (such that electrodes are implanted in the upper dermal region of the skin) that provides numerous benefits. These benefits include access to dermal interstitial fluid including many potential biomolecules that may serve as an analyte for detection.

Furthermore, when the microneedle array rests in the upper dermal region, the lower dermis beneath the microneedle array includes very high levels of vascularization and perfusion to support the dermal metabolism, which enables thermoregulation (via vasoconstriction and/or vasodilation) and provides a barrier function to help stabilize the sensing environment around the microneedles. Yet another advantage of the shallower insertion depth is that the upper dermal layers lack pain receptors, thus resulting in a reduced pain sensation when the microneedle array punctures the skin of the user, and providing for a more comfortable, minimally-invasive user experience.

Additionally, because of the shallower microneedle insertion depth of the analyte monitoring device 110, a reduced time delay in analyte detection is obtained compared to traditional continuous analyte monitoring devices. Such a shallower insertion depth positions the sensor surfaces in close proximity (e.g., within a few hundred micrometers or less) to the dense and well-perfused capillary bed of the reticular dermis, resulting in a negligible diffusional lag from the capillaries to the sensor surface. Diffusion time is related to diffusion distance according to t=x²/(2D) where is the diffusion time, x is the diffusion distance, and D is the mass diffusivity of the analyte of interest. Therefore, positioning an analyte sensing element twice as far away from the source of an analyte in a capillary will result in a quadrupling of the diffusional delay time. Accordingly, conventional analyte sensors, which reside in the very poorly vascularized adipose tissue beneath the dermis, result in a significantly greater diffusion distance from the vasculature in the dermis and thus a substantial diffusional latency (e.g., typically 5-20 minutes). In contrast, the shallower microneedle insertion depth of the analyte monitoring device 110 benefits from low diffusional latency from capillaries to the sensor, thereby reducing time delay in analyte detection and providing more accurate results in real-time or near real-time. For example, in some embodiments, diffusional latency may be less than 10 minutes, less than 5 minutes, or less than 3 minutes.

Furthermore, when the microneedle array rests in the upper dermal region, the lower dermis beneath the microneedle array includes very high levels of vascularization and perfusion to support the dermal metabolism, which enables thermoregulation (via vasoconstriction and/or vasodilation) and provides a barrier function to help stabilize the sensing environment around the microneedles. Yet another advantage of the shallower insertion depth is that the upper dermal layers lack pain receptors, thus resulting in a reduced pain sensation when the microneedle array punctures the skin of the user, and providing for a more comfortable, minimally-invasive user experience.

Thus, the analyte monitoring devices and methods described herein enable improved continuous monitoring of analyte of a user. For example, as described above, the analyte monitoring device may be simple and straightforward to apply, which improves ease-of-use and user compliance. Additionally, analyte measurements of dermal interstitial fluid may provide for highly accurate analyte detection. Furthermore, compared to traditional continuous analyte monitoring devices, insertion of the microneedle array and its sensors may be less invasive and involve less pain for the user. Additional advantages of other aspects of the analyte monitoring devices and methods are further described below.

### Housing

As described above, an analyte monitoring device may include a housing. The housing may at least partially surround or enclose other components of the analyte monitoring device (e.g., electronic components), such as for protection of such components. For example, the housing may be configured to help prevent dust and moisture from entering the analyte monitoring device. In some variations, an adhesive layer may attach the housing to a surface (e.g., skin) of a user, while permitting a microneedle array to extend outwardly from the housing and into the skin of the user. Furthermore, in some variations the housing may generally include rounded edges or corners and/or be low-profile so as to be atraumatic and reduce interference with clothing, etc. worn by the user.

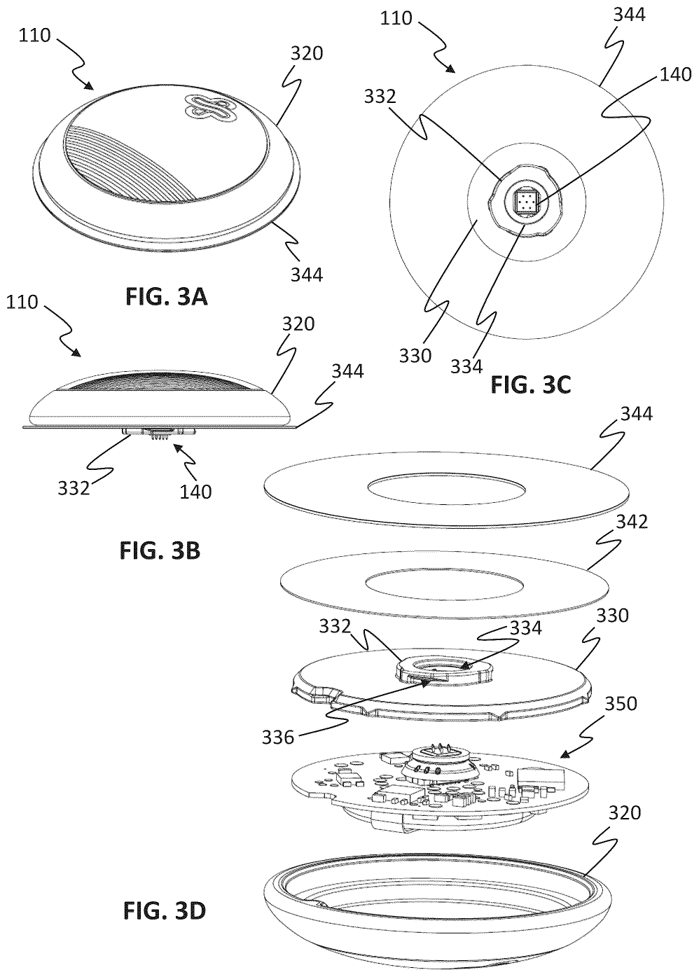

FIG. 3A-FIG. 3D depict aspects of the analyte monitoring device 110. FIGS. 3A-3D depict an upper perspective view, a side view, a bottom view, and an exploded view, respectively, of the analyte monitoring device 110.

The analyte monitoring device 110 may include a housing that at least partially surrounds or encloses other components (e.g., electronic components) of the analyte monitoring device 110, such as for protection of such components. For example, the housing may be configured to help prevent dust and moisture from entering the analyte monitoring device 110. In some variations, an adhesive layer may attach the housing to a surface (e.g., skin) of a user, while permitting the microneedle array 140 to extend outwardly from the housing and into the skin of the user. Furthermore, in some variations, the housing may generally include rounded edges or corners and/or be low-profile to reduce interference with clothing, etc. worn by the user.

For example, as shown in FIGS. 3A-3D, an example variation of the analyte monitoring device 110 may include a housing cover 320 and a base plate 330, configured to at least partially surround internal components of the analyte monitoring device 110. For example, the housing cover 320 and the base plate 330 may provide an enclosure for a sensor assembly 350 including the microneedle array 140 and electronic components. Once assembled, the microneedle array 140 extends outwardly from a portion of the base plate 330 in a skin-facing direction (e.g., an underside) of the analyte monitoring device 110.

The housing cover 320 and the base plate 330 may, for example, include one or more rigid or semi-rigid protective shell components that may couple together via suitable fasteners (e.g., mechanical fasteners), mechanically interlocking or mating features, and/or an engineering fit. The housing cover 320 and the base plate 330 may include radiused edges and corners and/or other atraumatic features. When coupled together, the housing cover 320 and the base plate 330 may form an internal volume that houses internal components, such as the sensor assembly 350. For example, the internal components arranged in the internal volume may be arranged in a compact, low-profile stack-up as the sensor assembly 350.

The analyte monitoring device 110 may include one or more adhesive layers to attach the analyte monitoring device 110 (e.g., the coupled together housing cover 320 and the base plate 330) to a surface (e.g., the skin) of a user. As shown in FIG. 3D, the one or more adhesive layers may include an inner adhesive layer 342 and an outer adhesive layer 344. The inner adhesive layer 342 may adhere to the base plate 330, and the outer adhesive layer 344 may adhere to the inner adhesive layer 342 and, on its outward facing side, provide an adhesive for adhering (e.g., temporarily) to the skin of the user. The inner adhesive layer 342 and the outer adhesive layer 344 together act as a double-sided adhesive for adhering the analyte monitoring device 110 to the skin of the user. The outer adhesive layer 344 may be protected by a release liner that the user removes to expose the adhesive prior to skin application. In some variations, a single adhesive layer is provided. In some variations, the outer adhesive layer 344, the inner adhesive layer 342, and/or the single adhesive layer may have a perimeter that extends farther than the perimeter or periphery of the housing cover 320 and the base plate 330. This may increase surface area for attachment and increase stability of retention or attachment to the skin of the user. The inner adhesive layer 342, the outer adhesive layer 344, and/or the single adhesive layer each have an opening that permits passage of the outwardly extending microneedle array 140, as further described below. The openings of the inner adhesive layer 342 and the outer adhesive layer 344 may generally align with one another but may, in some variations, differ in size such that one opening is smaller than the other opening. In some variations, the openings are substantially the same size.

The base plate 330 has a first surface (e.g., an outwardly exposed surface) opposite a second surface and serves as a support and/or connection structure and as a protective cover for the sensor assembly 350. The base plate 330 is sized and shaped to attach to the housing cover 320. The base plate 330 may be shaped to securely fit within the housing cover 320 such that outer edges of the base plate 330 align with corresponding edges of an opening of the housing 320. The alignment may be such that there is no gap between the outer edges of the base plate 330 and the corresponding edges of the opening of the housing cover 320.

A connection member 332 may be formed in a central or near central region of the first surface of the base plate 330. The connection member 332 is a protrusion (e.g., a projected hub) with sidewalls that extend from the first surface of the base plate 330 and with a first surface substantially parallel to the first surface of the base plate 330. Sidewalls extend from edges of the first surface of the connection member 332 to the first surface of the base plate 330. A remaining portion of the first surface of the base plate 330 surrounding the connection member 332 may be flat or substantially flat. One or more connector features 336 extend outwardly from the sidewalls of the connection member 332 to releasably engage with corresponding connectors of a microneedle enclosure, as further described below. The first surface and the sidewalls of the connection member 332 define, in part, a cavity. The cavity may be further defined through a portion of the base plate 330 adjacent (e.g., below) the connection member 332. The cavity has an opening, and is accessible, on the second surface of the base plate 330. An aperture 334 is formed through the first surface of the connection member 332. The aperture 334 may be sized and shaped such that the microneedle array 140 fits securely within and extends through the aperture 334. For example, sidewalls of the microneedle array 140 may align with corresponding sidewalls of the aperture 334. In some variations, the aperture 334 may be sized and shaped to correspond with an area surrounding the microneedle array 140. The openings in the inner adhesive layer 342 and the outer adhesive layer 344 (or the single adhesive layer) are sized such that the connection member 332 extends through the openings without interference with the adhesive layers. For example, the diameter of the opening of the inner adhesive layer 342 and the diameter of the opening of the outer adhesive layer 344 is larger than that of the connection member 332. In some variations, the opening of the inner adhesive layer 342 and/or the opening of the outer adhesive layer 344 (or that of the single adhesive layer) is in proximity with the sidewalls of the connection member 332 with a clearance to accommodate the one or more connector features 336. In some variations, one or more slits or notches may be formed in the inner adhesive layer 342, the outer adhesive layer 344, and/or the single adhesive layer, extending from the opening to aid in placement of the respective adhesive layer.

Although the housing cover 320 and the base plate 330 depicted in FIGS. 3A-3D are substantially circular with the housing cover 320 having a dome shape, in other variations, the housing cover 320 and the base plate 330 may have any suitable shape. For example, in other variations the housing cover 320 and the base plate 330 may be generally prismatic and have an elliptical, triangular, rectangular, pentagonal, hexagonal, or other suitable shape. The outer adhesive layer 344 (or the single adhesive layer) may extend outwardly from the housing cover 320 and the base plate 330 to extend beyond the perimeter of the housing cover 320. The outer adhesive layer 344 (or the single adhesive layer) may be circular, as shown in FIGS. 3A-3D or may have an elliptical, triangular, rectangular, pentagonal, hexagonal, or other suitable shape and need not be the same shape as the housing cover 320 and/or the base plate 330.

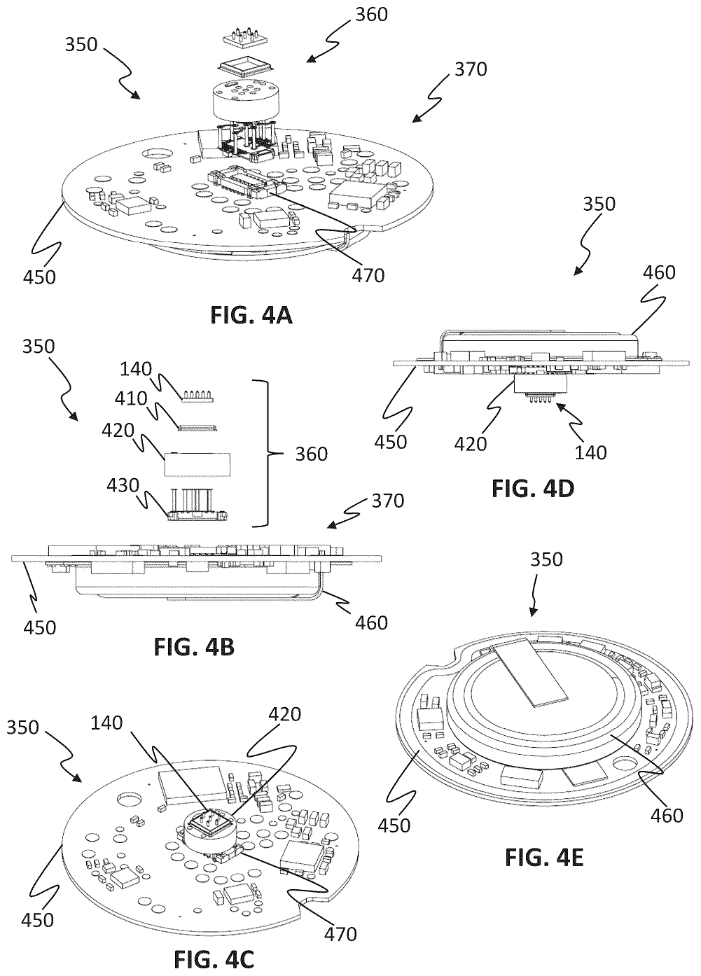

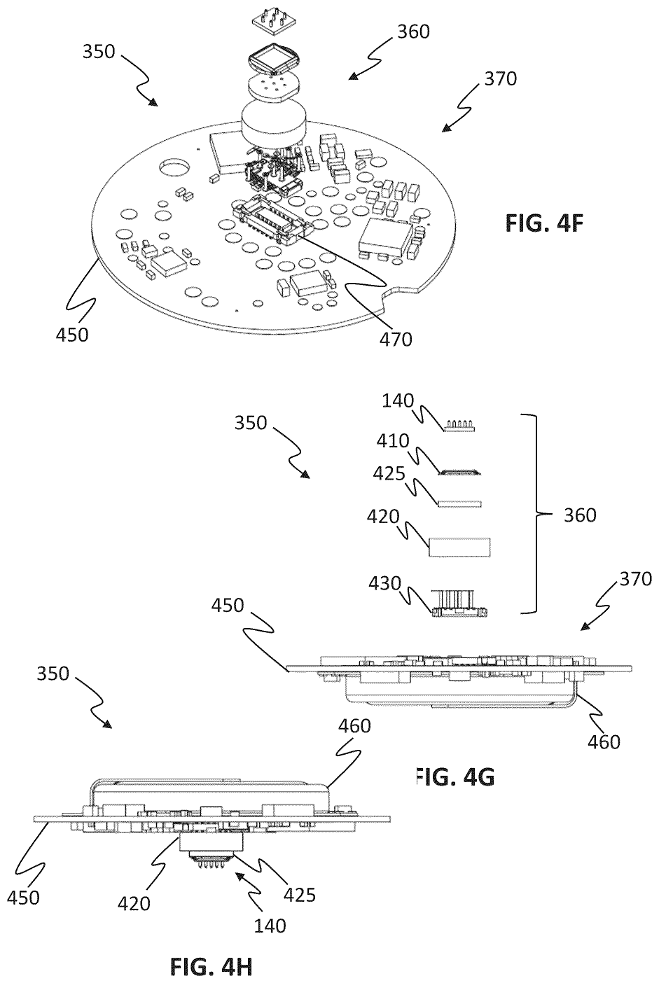

FIGS. 4A-4E depict aspects of the sensor assembly 350 of the analyte monitoring device 110 in a perspective exploded view, a side exploded view, a lower perspective view, a side view, and an upper perspective view, respectively.

The sensor assembly 350 includes microneedle array components and electronic components to implement analyte detection and processing aspects of the microneedle array-based continuous analyte monitoring device 110 for the detection and measuring of an analyte. In some variations, the sensor assembly 350 is a compact, low-profile stack-up that is at least partially contained within the internal volume defined by the housing cover 320 and the base plate 330.

In some variations, the sensor assembly 350 includes a microneedle array assembly 360 and an electronics assembly 370 that connect to one another to implement the microneedle array analyte detection and processing aspects further described herein. In some variations, the electronics assembly 370 includes a main printed circuit board (PCB) 450 on which electronic components are connected, and the microneedle array assembly 360 includes a secondary printed circuit board (PCB) 420 on which the microneedle array 140 is connected.

In some variations, the microneedle array assembly 360 includes, in addition to the secondary PCB 420 and the microneedle array 140, an epoxy skirt 410 and a secondary PCB connector 430. The microneedle array 140 is coupled to a top side (e.g., outer facing side) of the secondary PCB 420 so that the individual microneedles of the microneedle array 140 are exposed as described with reference to FIG. 3A-FIG. 3D. The secondary PCB connector 430 is coupled to a back side, opposite the top side, of the secondary PCB 420. The secondary PCB connector 430 may be an electromechanical connector and may communicatively couple to the primary PCB 450 through a primary PCB connector 470 on a top side (e.g., outer facing side) of the primary PCB 450 to allow for signal communication between the secondary PCB 420 and the primary PCB 450. For example, signals from the microneedle array 140 may be communicated to the primary PCB 450 through the secondary PCB 420, the secondary PCB connector 430, and the primary PCB connector 470.

The secondary PCB 420 may in part determine the distance to which the microneedle array 140 protrudes from the back plate 330 of the housing. Accordingly, the height of the secondary PCB 420 may be selected to help ensure that the microneedle array 140 is inserted properly into a user's skin. During microneedle insertion, the first surface (e.g., outer facing surface) of the connection member 332 of the back plate 330 may act as a stop for microneedle insertion. If the secondary PCB 420 has a reduced height and its top surface is flush or nearly flush with the first surface of the connection member 332, then the connection member 332 may prevent the microneedle array 140 from being fully inserted into the skin.

In some variations, other components (e.g., electronic components such as sensors or other components) may also be connected to the secondary PCB 420. For example, the secondary PCB 420 may be sized and shaped to accommodate electronic components on the top side or the back side of the secondary PCB 420.

In some variations, the epoxy skirt 410 may be deposited along the edges (e.g., the outer perimeter) of the microneedle array 140 to provide a secure fit of the microneedle array 140 within the aperture 334 formed in the connection member 332 of the base plate 330 and/or to relieve the sharp edges along the microneedle array 140, as shown in FIG. 3C and FIG. 3D. For example, the epoxy skirt 410 may occupy portions of the aperture 334 not filled by the microneedle array 140 and/or portions of the cavity defined in the base plate 330 not filled by the secondary PCB 420. The epoxy skirt 410 may also provide a transition from the edges of the microneedle array 140 to the edge of the secondary PCB 420. In some variations, the epoxy skirt 410 may be replaced or supplemented by a gasket (e.g., a rubber gasket) or the like.

The electronics assembly 370, having the primary PCB 450, includes a battery 460 coupled to a back side of the primary PCB 450, opposite the top side on which the primary PCB connector 470 is coupled. In some variations, the battery 460 may be coupled on the top side of the primary PCB 450 and/or in other arrangements.

FIGS. 4F-4H depict aspects of an alternate variation of the sensor assembly 350 of the analyte monitoring device 110. A perspective exploded view, a side exploded view, and a side view of the sensor assembly 350 are provided, respectively, in FIGS. 4F-4H.

As shown, in the sensor assembly 350, an additional PCB component, an intermediate PCB 425, is incorporated. In some variations, the intermediate PCB 425 is part of the microneedle array assembly 360 and is positioned between and connected to the secondary PCB 420 and the microneedle array 140. The intermediate PCB 425 may be added to increase the height of the microneedle array assembly 360 such that the microneedle array 140 extends at a further distance from the base plate 330, which may aid in insertion of the microneedle array 140 into the skin of a user. The microneedle array 140 is coupled to a top side (e.g., outer facing side) of the intermediate PCB 425 so that the individual microneedles of the microneedle array 140 are exposed as described with reference to FIG. 3A through FIG. 3D. The secondary PCB 420 is coupled to a back side, opposite the top side, of the intermediate PCB 425, and the secondary PCB connector 430 is coupled to a back side, opposite the top side, of the secondary PCB 420. The epoxy skirt 410 (which may be replaced or supplemented by a gasket of the like) provides a transition from the edges of the microneedle array 140 to the edge of the intermediate PCB 425.

The intermediate PCB 425 with the secondary PCB 420 in part determine the distance to which the microneedle array 140 protrudes through the aperture 334 of the back plate 330. The incorporation of the intermediate PCB 425 provides an additional height to help ensure that the microneedle array 140 is properly inserted into a user's skin. In some variations, the top side (e.g., outer facing side) of the intermediate PCB 425 extends through and out of the aperture 334 so that the first surface (e.g., top, exposed surface) of the connection member 332 surrounding the aperture 334 does not prevent the microneedle array from being fully inserted into the skin. In some variations, the top side (e.g., outer facing side) of the intermediate PCB 425 does not extend out of the aperture 334 but the increased height (by virtue of incorporating the intermediate PCB 425) ensures that the microneedle array 140 protrudes at a sufficient distance from the back plate 330 of the housing.

In some variations, a microneedle enclosure may be provided for releasable attachment to the analyte monitoring device 110. The microneedle enclosure may provide a protective environment or enclosure in which the microneedle array 140 may be safely contained, thereby ensuring the integrity of the microneedle array 140 during certain stages of manufacture and transport of the analyte monitoring device 110, prior to application of the analyte monitoring device 110. The microneedle enclosure is releasable or removable from the analyte monitoring device 110 to allow for the microneedle array 140 to be exposed and ready for insertion into the skin of the user, as further described herein.

In some variations, the microneedle enclosure, by providing an enclosed and sealed environment in which the microneedle array 140 may be contained, provides an environment in which the microneedle array 140 may be sterilized. For example, the microneedle enclosure with the microneedle array 140 may be subjected to a sterilization process, during which the sterilization penetrates the microneedle enclosure so that the microneedle array 140 is also sterilized. As the microneedle array 140 is contained in an enclosed environment, the microneedle array 140 remains sterilized until removed from the enclosed environment.

### User Interface

In some variations, an analyte monitoring system may provide user status, analyte monitoring device status, and/or other suitable information directly via a user interface (e.g., display, indicator lights, etc. as described below) on the analyte monitoring device. Thus, in contrast to analyte monitoring systems that may solely communicate information to a separate peripheral device (e.g., mobile phone, etc.) that in turn communicates the information to a user, in some variations such information may be directly provided by the analyte monitoring device. Advantageously, in some variations, such a user interface on the analyte monitoring device may reduce the need for a user to constantly maintain a separate peripheral device in order to monitor user status and/or analyte monitoring device status (which may be impractical due to cost, inconvenience, etc.). Additionally, the user interface on the analyte monitoring device may reduce risks associated with loss of communication between the analyte monitoring device and a separate peripheral device, such as a user having an inaccurate understanding of their current analyte levels (e.g., leading the user to assume their analyte levels are high when they are actually low, which could, for example, result in the user self-administering an inaccurate dose of drug or withholding a therapeutic intervention when it is medically necessary).

Additionally, the ability to communicate information to a user via the analyte monitoring device itself, independently of a separate peripheral device, may reduce or eliminate the need to maintain compatibility between the analyte monitoring device and separate peripheral devices as such peripheral devices are upgraded (e.g., replaced with new device models or other hardware, run new versions of operating systems or other software, etc.).

Accordingly, in some variations, the housing may include a user interface, such as an interface to provide information in a visual, audible, and/or tactile manner to provide information regarding user status based on analyte measurements and/or status of the analyte monitoring device, and/or other suitable information.

Examples of user status based on analyte measurements that may be communicated via the user interface include information representative of analyte measurement in the user, such as: analyte concentration in a bodily fluid such as dermal interstitial fluid or bloodstream; the analyte measurements being below a predetermined analyte measurement threshold or range, within a predetermined analyte measurement range, or above a predetermined analyte measurement threshold or range; increase or decrease of analyte measurement over time; rate of change of analyte measurement; analyte variability indicating a standard deviation of analyte measurements during a time period; information relating to trends of analyte measurements; and/or other suitable alerts associated with analyte measurement.

Examples of analyte monitoring device status that may be communicated via the user interface include device operation mode (e.g., associated with device warm-up state, analyte monitoring state, battery power status such as low battery, etc.), a device error state (e.g., operational error, pressure-induced sensing attenuation, fault, failure mode, etc.), device power status, device life status (e.g., anticipated sensor end-of-life), status of connectivity between device and a mobile computing device, and/or the like.

In some variations, the user interface may by default be in an enabled or “on” state to communicate such information at least whenever the analyte monitoring device is performing analyte measurements) or whenever the analyte monitoring device is powered on, thereby helping to ensure that information is continuously available to the user. For example, user interface elements may communicate through a display or indicator light(s) (e.g., as described below) not only alerts to flag user attention or recommend remedial action, but also when user status and/or device status are normal. Accordingly, in some variations, a user is not required to perform an action to initiate a scan to learn their current analyte measurement level(s), and such information may always readily be available to the user. In some variations, however, a user may perform an action to disable the user interface temporarily (e.g., similar to a “snooze” button) such as for a predetermined amount of time (e.g., 30 minutes, 1 hour, 2 hours, etc.) after which the user interface is automatically reenabled, or until a second action is performed to reenable the user interface.

In some variations, the user interface of the housing may include a display configured to visually communicate information. The display may, for example, include a display screen (e.g., LCD screen, OLED display, electrophoretic display, electrochromic display, etc.) configured to display alphanumeric text (e.g., numbers, letters, etc.), symbols, and/or suitable graphics to communicate information to the user. For example, the display screen may include a numerical information, textual information, and/or a graphics (e.g., sloped line, arrows, etc.) of information such as user status and/or status of the analyte monitoring device. For example, the display screen may include text or graphical representations of analyte measurement levels, trends, and/or recommendations. For example, the display screen may include text and/or graphical representations related to recommendations for physical activity, meditation, rest, food, dietary supplements, and/or medical consultation.

As another example, the display on the housing may include one or more indicator lights (e.g., including LEDs, OLEDs, lasers, electroluminescent material, or other suitable light source, waveguides, etc.) that may be controlled in one or more predetermined illumination modes to communicate different statuses and/or other suitable information. An indicator light may be controlled to illuminate with multiple colors (e.g., red, orange, yellow, green, blue, and/or purple, etc.) or in only one color. For example, an indicator light may include a multi-colored LED. As another example, an indicator light may include a transparent or semi-transparent material (e.g., acrylic) positioned over one or more different-colored light sources (e.g., LED) such that different-colored light sources may be selectively activated to illuminate the indicator light in a selected color. The activation of light sources can either occur simultaneously or in sequence. An indicator light may have any suitable form (e.g., raised, flush, recessed, etc. from housing body) and/or shape (e.g., circle or other polygon, ring, elongated strip, etc.). In some variations, an indicator light may have a pinhole size and/or shape to present the same intensity of the light as a larger light source, but with significantly less power requirements, which may help conserve onboard power in the analyte monitoring device.

Indicator light(s) on the display may be illuminated in one or more various manners to communicate different kinds of information. For example, an indicator light may be selectively illuminated on or off to communicate information (e.g., illumination “on” indicates one status, while illumination “off” indicates another status). Additionally, or alternatively, an indicator light may be illuminated in a selected color or intensity to communicate information (e.g., illumination in a first color or intensity indicates a first status, while illumination in a second color or intensity indicates a second status). Additionally, or alternatively, an indicator light may be illuminated in a selected temporal pattern to communicate information (e.g., illumination in a first temporal pattern indicates a first status, while illumination in a second temporal pattern indicates a second status). For example, an indicator light may be selectively illuminated in one of a plurality of predetermined temporal patterns that differ in illumination frequency (e.g., repeated illumination at a rapid or slow frequency), regularity (e.g., periodic repeated illumination vs. intermittent illumination), duration of illumination “on” time, duration of illumination “off” time, rate of change in illumination intensity, duty cycle (e.g., ratio of illumination “on” time to illumination “off” time), and/or the like, where each predetermined temporal pattern may indicate a respective status.

Additionally, or alternatively, in some variations, a display may include multiple indicator lights that may be collectively illuminated in one or more predetermined illumination modes or sequences in accordance with one or more predetermined spatial and/or temporal patterns. For example, in some variations, some or all of the indicator lights arranged on a display may be illuminated in synchrony or in sequence to indicate a particular status. Accordingly, the selected subset of indicator lights (e.g., the spatial arrangement of the indicator lights that are illuminated) and/or the manner in which they are illuminated (e.g., illumination order, illumination rate, etc.) may indicate a particular status. Additionally, or alternatively, a plurality of indicator lights may illuminate simultaneously or in sequence to increase the diversity of the color palette. For example, in some variations, red, green, and blue LEDs may be illuminated in rapid succession to create the impression of white light to a user.

It should furthermore be understood that one or more of the above-described illumination modes may be combined in any suitable manner (e.g., combination of varying color, intensity, brightness, luminosity, contrast, timing, location, etc.) to communicate information. Additionally, or alternatively, an ambient light sensor may be incorporated into the device body to enable dynamic adjustment light levels in the indicator light(s) to compensate for environmental light conditions to help conserve power. The ambient light sensor may, in some variations, be used in conjunction with a kinetic sensor (e.g., as described in further detail below) to further determine appropriate periods for the analyte monitoring device to enter into a power saving mode or reduced power state. For example, detection of darkness and no motion of the analyte monitoring device may indicate that the wearer of the analyte monitoring device is asleep, which may trigger the analyte monitoring device to enter into a power saving mode or reduced power state.

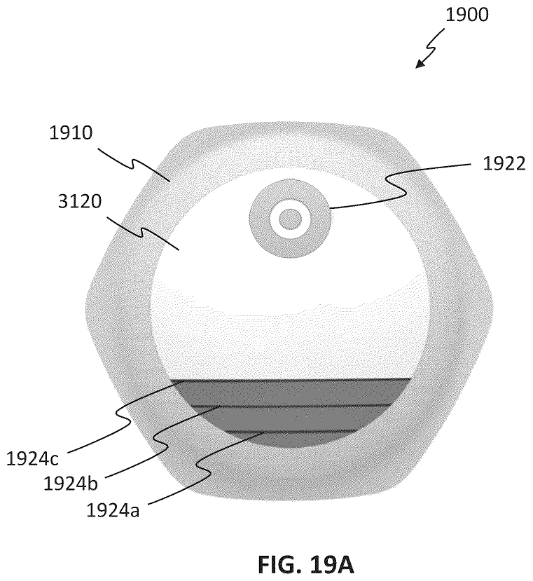

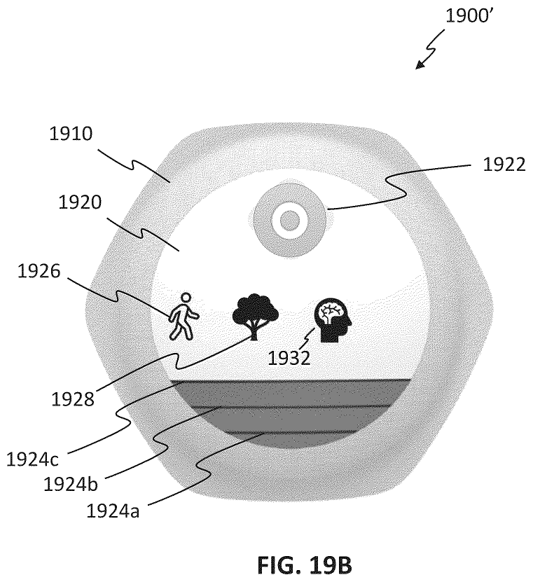

FIG. 19A illustrates an example variation of an analyte monitoring device 1900 including a user interface 1920 with multiple indicator lights (1922, 1924a-1924c). Indicator light 1922 may, for example, be selectively illuminated to indicate a device state (e.g., operation mode, error state, power status, life status, etc.). Although indicator light 1922 is in the shape of a symbol (e.g., logo), it should be understood that in other variations, the indicator light 1922 may have any suitable shape (e.g., text, other geometric shape, etc.). Indicator lights 1924a, 1924b, 1924c may be selectively illuminated to indicate a user status (e.g., information representative of analyte measurement). Although indicator lights 1924a, 1924b, 1924c are linear elements extending across the user interface (e.g., chords across a circular display), it should be understood that in other variations, the indicator lights 1924a, 1924b, 1924c have other suitable shapes (e.g., wavy lines, circular, etc.). In some variations, a 1-dimensional array of indicator lights of any suitable shape may be arranged on the housing (e.g., arranged in a row, a column, an arc, etc.). Alternatively, the housing may include a multi-dimensional array of indicator lights of any suitable shape.

Furthermore, in some variations, an indicator light may include an icon (e.g., symbol) that may be indicative of analyte information (e.g., up arrow to indicate rising analyte measurement level trend, down arrow to indicate falling analyte measurement level trend), analyte monitoring device status (e.g., exclamation point to indicate a device error state), and/or other suitable information. Additionally, or alternatively, iconography in the indicator light(s) may be used to communicate recommendations for the user such as behavioral recommendations. Iconography may, for example, have the advantage of communicating recommendations to a user in a more universal or language-agnostic manner (e.g., without the need for language translations to tailor the device to different geographical regions or user preferences, etc.). In an example, rising analyte levels may be correlated to an increase in user stress. For example, as shown in FIG. 19B, in some variations, a user interface for an analyte monitoring device 1900′ may include a running person icon 1926 to indicate a recommendation that the user engage in physical activity, a tree icon 3128 to indicate a recommendation that the user step outside, and/or a thinking head icon 1932 to indicate a recommendation that the user meditate.

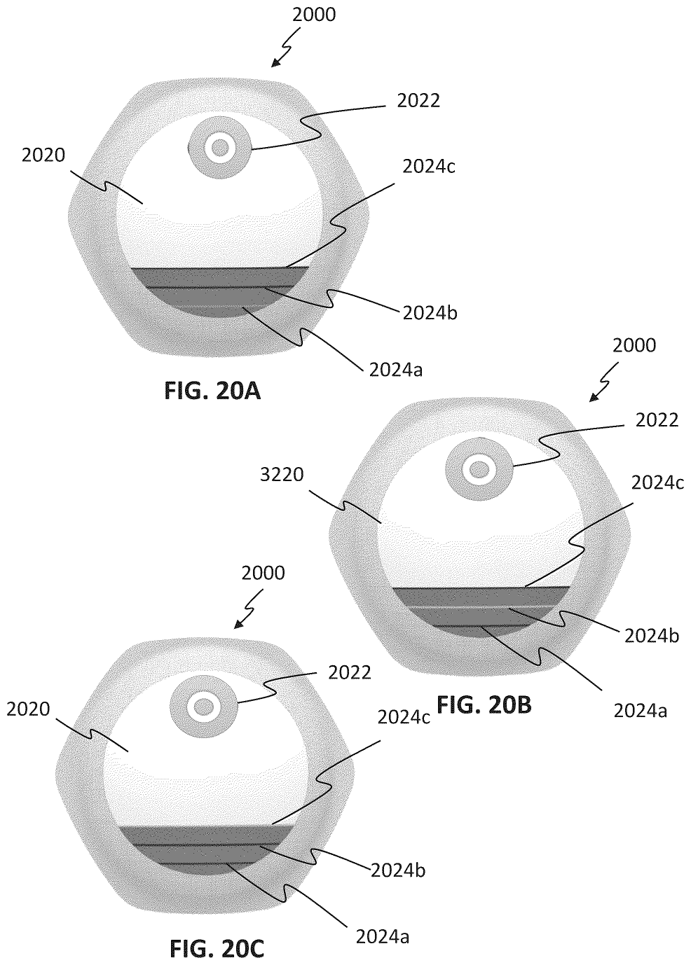

In the variations shown in FIGS. 19A and 19B, each of the indicator lights 1924a, 1924b, 1924c may be exclusively illuminated to indicate a different analyte measurement (e.g., in target range, below target range, significantly below target range, above target range, significantly above target range, etc.). Furthermore, the indicator lights 1924a, 1924b, 1924c may be arranged adjacent to each other, such that they may be selectively illuminated in a progressive sequence to communicate trend information of analyte measurements (e.g., progressive sequence of illumination in a first direction that corresponds to an increase in measured quantity of analyte, progressive sequence of illumination in a second direction that corresponds to a decrease in measured quantity of analyte, pace of illumination progression in the first direction or the second direction that corresponds to a rate of increase or decrease in measured quantity of analyte, etc.). While one device status indicator light 1922 and three user status indicator lights 1924a, 1924b, 1924c are shown in FIGS. 19A and 19B, it should be understood that in other variations, an analyte monitoring device may include any suitable number of indicator lights, such as one, two, three, four, five or more device status indicator lights, and one, two, three, four, five or more user status indicator lights. Further details regarding an example operation of the user interface 1920 to communicate device status and/or user status are described below (e.g., with reference to FIGS. 20A-20C).

### Microneedle Array

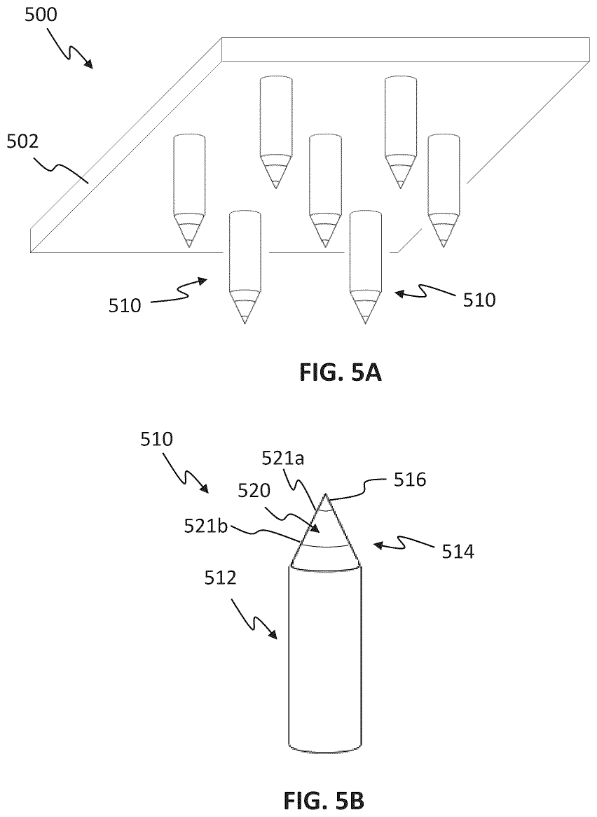

As shown in the schematic of FIG. 5A, in some variations, a microneedle array 510 for use in sensing an analyte may include one or more microneedles 510 projecting from a substrate surface 502. The substrate surface 502 may, for example, be a generally planar semiconductor (e.g., silicon) substrate and one or more microneedles 510 may project orthogonally from the planar surface. Generally, as shown in FIG. 5B, a microneedle 510 may include a body portion 512 (e.g., shaft) and a tapered distal portion 514 configured to puncture skin of a user. In some variations, the tapered distal portion 514 may terminate in an insulated distal apex 516. The microneedle 510 may further include an electrode 520 on a surface of the tapered distal portion. In some variations, electrode-based measurements may be performed at the interface of the electrode and interstitial fluid located within the body (e.g., on an outer surface of the overall microneedle). In some variations, the microneedle 510 may have a solid core (e.g., solid body portion), though in some variations the microneedle 510 may include one or more lumens, which may be used for drug delivery or sampling of the dermal interstitial fluid, for example. Other microneedle variations, such as those described below, may similarly either include a solid core or one or more lumens.

The microneedle array 500 may be at least partially formed from a semiconductor (e.g., silicon) substrate and include various material layers applied and shaped using various suitable microelectromechanical systems (MEMS) manufacturing techniques (e.g., deposition and etching techniques), as further described below. The microneedle array may be reflow-soldered to a circuit board, similar to a typical integrated circuit. Furthermore, in some variations the microneedle array 500 may include a three-electrode setup including a working (sensing) electrode having an electrochemical sensing coating (including a biorecognition element such as an aptamer or an enzyme) that enables detection of the analyte, a reference electrode, and a counter electrode. In other words, the microneedle array 500 may include at least one microneedle 510 that includes a working electrode, at least one microneedle 510 including a reference electrode, and at least one microneedle 510 including a counter electrode. Additional details of these types of electrodes are described in further detail below.

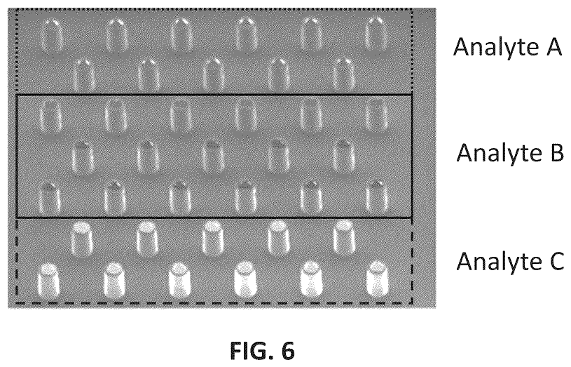

In some variations, the microneedle array 500 may include a plurality of microneedles that are insulated such that the electrode on each microneedle in the plurality of microneedles is individually addressable and electrically isolated from every other electrode on the microneedle array. The resulting individual addressability of the microneedle array 500 may enable greater control over each electrode's function, because each electrode may be separately probed. For example, the microneedle array 500 may be used to provide multiple independent measurements of a given analyte, which improves the device's sensing reliability and accuracy. Furthermore, in some variations the electrodes of multiple microneedles may be electrically connected to produce augmented signal levels. As another example, the same microneedle array 500 may additionally or alternatively be interrogated to simultaneously measure multiple analytes to provide a more comprehensive assessment of physiological status. For example, as shown in the schematic of FIG. 6, a microneedle array may include a portion of microneedles to detect s first analyte A, a second portion of microneedles to detect a second Analyte B, and a third portion of microneedles to detect a third Analyte C. It should be understood that the microneedle array may be configured to detect any suitable number of analytes (e.g., 1, 2, 3, 4, 5 or more, etc.), provided that at least one of the analytes is analyte.

In some variations of microneedles (e.g., microneedles with a working electrode), the electrode 520 may be located proximal to the insulated distal apex 516 of the microneedle. In other words, in some variations the electrode 520 does not cover the apex of the microneedle. Rather, the electrode 520 may be offset from the apex or tip of the microneedle. The electrode 520 being proximal to or offset from the insulated distal apex 516 of the microneedle advantageously provides more accurate sensor measurements. For example, this arrangement prevents concentration of the electric field at the microneedle apex 516 during manufacturing, thereby avoiding non-uniform electro-deposition of sensing chemistry on the electrode surface 520 that would result in faulty sensing. The electrode 520 may be configured to have an annular shape and may comprise a distal edge 521a and a proximal edge 521b.

As another example, placing the electrode 520 offset from the microneedle apex further improves sensing accuracy by reducing undesirable signal artefacts and/or erroneous sensor readings caused by stress upon microneedle insertion. The distal apex of the microneedle is the first region to penetrate into the skin, and thus experiences the most stress caused by the mechanical shear phenomena accompanying the tearing or cutting of the skin. If the electrode 520 were placed on the apex or tip of the microneedle, this mechanical stress may delaminate the electrochemical sensing coating on the electrode surface when the microneedle is inserted, and/or cause a small yet interfering amount of tissue to be transported onto the active sensing portion of the electrode. Thus, placing the electrode 520 sufficiently offset from the microneedle apex may improve sensing accuracy. For example, in some variations, a distal edge 521a of the electrode 520 may be located at least about 10 μm (e.g., between about 20 μm and about 30 μm) from the distal apex or tip of the microneedle, as measured along a longitudinal axis of the microneedle.

The body portion 512 of the microneedle 510 may further include an electrically conductive pathway extending between the electrode 520 and a backside electrode or other electrical contact (e.g., arranged on a backside of the substrate of the microneedle array). The backside electrode may be soldered to a circuit board, enabling electrical communication with the electrode 520 via the conductive pathway. For example, during use, the in-vivo sensing current (inside the dermis) measured at a working electrode is interrogated by the backside electrical contact, and the electrical connection between the backside electrical contact and the working electrode is facilitated by the conductive pathway. In some variations, this conductive pathway may be facilitated by a metal via running through the interior of the microneedle body portion (e.g., shaft) between the microneedle's proximal and distal ends. Alternatively, in some variations the conductive pathway may be provided by the entire body portion being formed of a conductive material (e.g., doped silicon). In some of these variations, the complete substrate on which the microneedle array 500 is built upon may be electrically conductive, and each microneedle 510 in the microneedle array 500 may be electrically isolated from adjacent microneedles 510 as described below. For example, in some variations, each microneedle 510 in the microneedle array 500 may be electrically isolated from adjacent microneedles 510 with an insulative barrier including electrically insulative material (e.g., dielectric material such as silicon dioxide) that surrounds the conductive pathway extending between the electrode 520 and backside electrical contact. For example, body portion 512 may include an insulative material that forms a sheath around the conductive pathway, thereby preventing electrical communication between the conductive pathway and the substrate. Other example variations of structures enabling electrical isolation among microneedles are described in further detail below.

Such electrical isolation among microneedles in the microneedle array permits the sensors to be individually addressable. This individually addressability advantageously enables independent and parallelized measurement among the sensors, as well as dynamic reconfiguration of sensor assignment (e.g., to different analytes). In some variations, the electrodes in the microneedle array can be configured to provide redundant analyte measurements, which is an advantage over conventional analyte monitoring devices. For example, redundancy can improve performance by improving accuracy (e.g., averaging multiple analyte measurement values from different microneedles which reduces the effect of extreme high or low sensor signals on the determination of analyte levels) and/or improving reliability of the device by reducing the likelihood of total failure.

In some variations, as described in further detail below with respective different variations of the microneedle, the microneedle array may be formed at least in part with suitable semiconductor and/or MEMS fabrication techniques and/or mechanical cutting or dicing. Such processes may, for example, be advantageous for enabling large-scale, cost-efficient manufacturing of microneedle arrays.

### Microneedle Structures

Described herein are further example variations of microneedle structures incorporating one or more of the above-described microneedle features for a microneedle array in an analyte monitoring device.

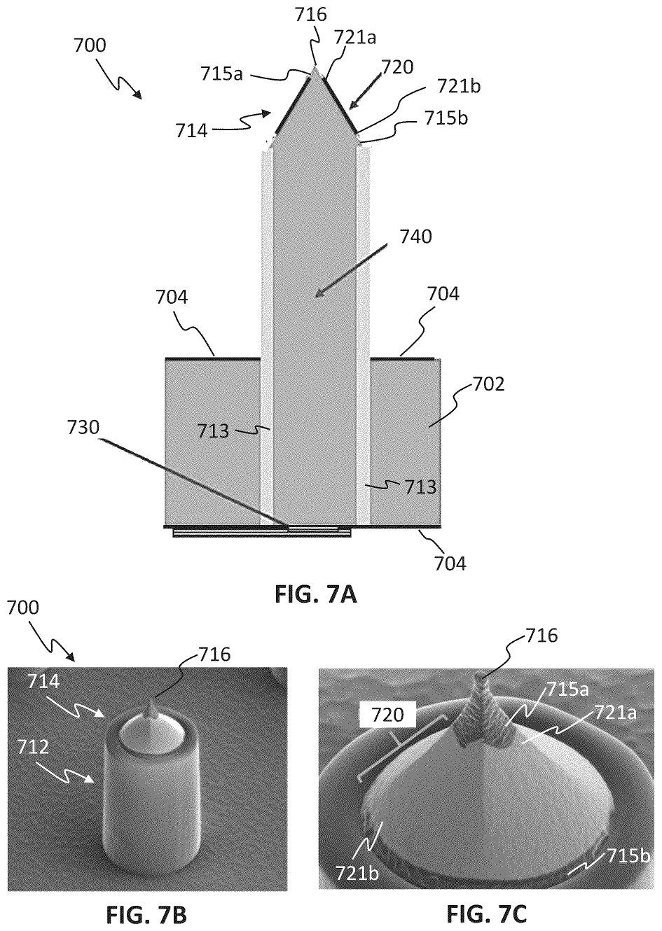

In some variations, a microneedle may have a generally columnar body portion and a tapered distal portion with an electrode. For example, FIGS. 7A-4C illustrate an example variation of a microneedle 700 extending from a substrate 702. FIG. 7A is a side cross-sectional view of a schematic of microneedle 700, while FIG. 7B is a perspective view of the microneedle 700 and FIG. 7C is a detailed perspective view of a distal portion of the microneedle 700. As shown in FIGS. 7B and 7C, the microneedle 700 may include a columnar body portion 712, a tapered distal portion 714 terminating in an insulated distal apex 716, and an annular electrode 720. The annular electrode 720 includes a conductive material (e.g., Pt, Ir, Au, Ti, Cr, Ni, combinations thereof, etc.) arranged on the tapered distal portion 714, such as, for example, on a segment thereof, and comprises a distal edge 721a and a proximal edge 721b. As shown in FIG. 7A, the annular electrode 720 may be proximal to (offset or spaced apart from) the distal apex 716. The annular electrode 720 may be electrically isolated from the distal apex 716 by a distal insulating surface 715a including an insulating material (e.g., SiO₂). For example, the distal edge 721a of the annular electrode 720 may be proximate to a proximal edge of the distal insulating surface 715a of the insulated distal apex 716. In some variations, the distal edge 721a of the annular electrode 720 may be proximal to (e.g., just proximal to, adjacent, abutting) a proximal edge of the distal apex 716 (a proximal edge of the distal insulating surface 715a), while in other variations, the distal edge 721a of the annular electrode 720 may be distal to (e.g., just distal to, adjacent) the proximal edge of the insulated distal apex 716 (proximal edge of the distal insulating surface 715a), but may remain proximal to the apex itself. Accordingly, in some variations, the annular electrode 720 may overlie a portion of the distal insulating surface 715a, but may remain proximal to (and offset from) the insulated distal apex itself.

Also as shown in FIG. 7A, the proximal edge 721b of the annular electrode 720 may be distal to, and in some variations, offset or spaced apart from, the columnar body portion 712. In some variations, the proximal edge 721b of the annular electrode 720 may also be electrically isolated from the columnar body portion 712 by a second distal insulating surface 715b comprising an insulating material (e.g., SiO₂) at a proximal end or region of the tapered distal portion 714. For example, the proximal edge 721b of the annular electrode 720 may be proximate to a distal edge of the second distal insulating surface 715b. In some variations, the proximal edge 721b of the annular electrode 720 may be proximal to (e.g., just proximal to, adjacent, abutting) a distal edge the second distal insulating surface 715b, while in other variations, the proximal edge 721b of the annular electrode 720 may be distal to (e.g., just distal to, adjacent) the distal edge of the second distal insulating surface 715b, but may remain proximal to the columnar body portion 712. Accordingly, in some variations, the annular electrode 720 may overlie a portion of the second distal insulating surface 715b but may remain proximal to (and offset from) the columnar body portion 712. As shown in FIG. 7A and in some other variations, the annular electrode 720 may be on only a segment of the surface of the tapered distal portion 714 and may or may not extend to the columnar boy portion 712.

The electrode 720 may be in electrical communication with a conductive core 740 (e.g., conductive pathway) passing along the body portion 712 to a backside electrical contact 730 (e.g., made of Ni/Au alloy) or another electrical pad in or on the substrate 702. For example, the body portion 712 may include a conductive core material (e.g., highly doped silicon). As shown in FIG. 7A, in some variations, an insulating moat 713 including an insulating material (e.g., SiO₂) may be arranged around (e.g., around the perimeter) of the body portion 712 and extend at least partially through the substrate 702. Accordingly, the insulating moat 713 may, for example, help prevent electrical contact between the conductive core 740 and the surrounding substrate 702. The insulating moat 713 may further extend over the surface of the body portion 712. Upper and/or lower surfaces of the substrate 702 may also include a layer of substrate insulation 704 (e.g., SiO₂). Accordingly, the insulation provided by the insulating moat 713 and/or substrate insulation 704 may contribute at least in part to the electrical isolation of the microneedle 700 that enables individual addressability of the microneedle 700 within a microneedle array. Furthermore, in some variations the insulating moat 713 extending over the surface of the body portion 712 may function to increase the mechanical strength of the microneedle 700 structure.

The microneedle 700 may be formed at least in part by suitable MEMS fabrication techniques such as plasma etching, also called dry etching. For example, in some variations, the insulating moat 713 around the body portion 712 of the microneedle may be made by first forming a trench in a silicon substrate by deep reactive ion etching (DRIE) from the backside of the substrate, then filling that trench with a sandwich structure of SiO₂/polycrystalline silicon (poly-Si)/SiO₂ by low pressure chemical vapor deposition (LPCVD) or other suitable process. In other words, the insulating moat 713 may passivate the surface of the body portion 712 of the microneedle and continue as a buried feature in the substrate 702 near the proximal portion of the microneedle. By including largely compounds of silicon, the insulating moat 713 may provide good fill and adhesion to the adjoining silicon walls (e.g., of the conductive core 740, substrate 702, etc.). The sandwich structure of the insulating moat 713 may further help provide excellent matching of coefficient of thermal expansion (CTE) with the adjacent silicon, thereby advantageously reducing faults, cracks, and/or other thermally-induced weaknesses in the insulating structure 713.

The tapered distal portion may be fashioned out by an isotropic dry etch from the frontside of the substrate, and the body portion 712 of the microneedle 700 may be formed from DRIE. The frontside metal electrode 720 may be deposited and patterned on the distal portion by specialized lithography (e.g., electron-beam evaporation) that permits metal deposition in the desired annular region for the electrode 720 without coating the distal apex 716. Furthermore, the backside electrical contact 730 of Ni/Au may be deposited by suitable MEMS manufacturing techniques (e.g., sputtering).

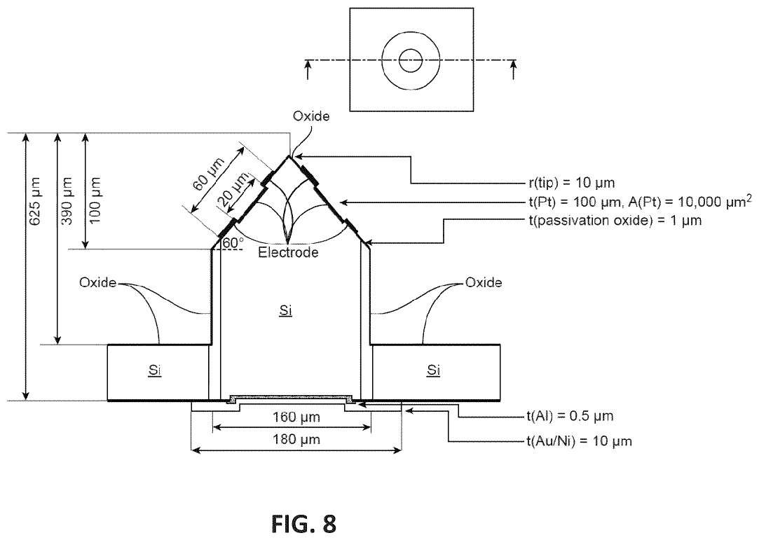

The microneedle 700 may have any suitable dimensions. By way of illustration, the microneedle 700 may, in some variations, have a height of between about 300 μm and about 500 μm. In some variations, the tapered distal portion 714 may have a tip angle between about 60 degrees and about 80 degrees, and an apex diameter of between about 1 μm and about 15 μm. In some variations, the surface area of the annular electrode 720 may include between about 9,000 μm² and about 11,000 μm², or about 10,000 μm². FIG. 8 illustrates various dimensions of an example variation of a columnar microneedle with a tapered distal portion and annular electrode, similar to microneedle 700 described above. As with the microneedle 700 described above, the columnar microneedle of FIG. 8 comprises a columnar body portion, a tapered distal portion terminating in an insulated distal apex, a contact trench formed within the tapered distal portion, and an annular electrode (denoted by “Pt” in FIG. 8) that is arranged on the tapered distal portion and overlays the contact trench. The annular electrode may comprise a conductive material (e.g., Pt, Ir, Au, Ti, Cr, Ni, combinations thereof, etc.). In some variations, the contact trench may have a width of about 1 μm (micrometer), about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, or, as shown in FIG. 8, about 20 μm. The annular electrode may comprise a distal edge and a proximal edge, and in some variations, a distance between the distal edge and the proximal edge of the annular electrode may be about 20 μm, about 30 μm, about 40 μm about 50 μm, about 60 μm, about 70 μm, about 80 μm, about 90 μm, about 100 μm, or, as shown in FIG. 8, about 60 μm. In some variations, and as shown in FIG. 8 by the dimensional callouts 60 μm and 20 μm, the annular electrode may overlie the contact trench and, in some instances, a portion of the insulating surfaces (denoted by “Oxide” in FIG. 8) of the tapered distal portion.

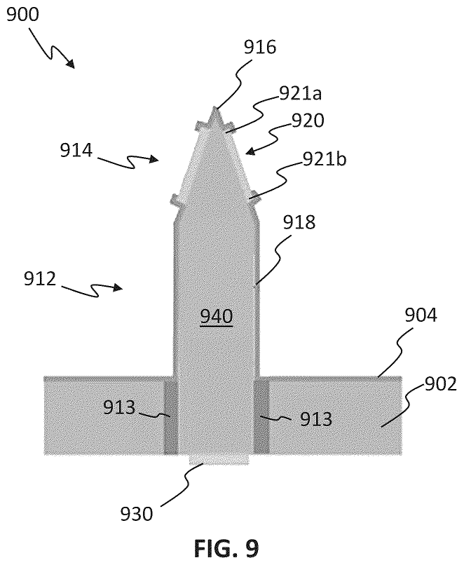

FIG. 9 illustrates another example variation of a microneedle 900 having a generally columnar body portion. The microneedle 900 may be similar to microneedle 700 as described above, except as described below. For example, like the microneedle 700, the microneedle 900 may include a columnar body portion 912, and a tapered distal portion 914 terminating in an insulated distal apex 916. The microneedle 900 may further include an annular electrode 920 that includes a conductive material and is arranged on the tapered distal portion 914 at a location proximal to (or offset from or spaced apart from) the distal apex 916. Other elements of microneedle 900 have numbering similar to corresponding elements of microneedle 700.

However, compared to the microneedle 700, the microneedle 900 may have a sharper tip at the distal apex 916 and a modified insulating moat 913. For example, the distal apex 916 may have a sharper tip angle, such as between about 25 degrees and about 45 degrees, and an apex radius of less than about 100 nm, which provides a sharper microneedle profile that may penetrate skin with greater ease, lower velocity, less energy, and/or less trauma. Furthermore, in contrast to the insulating moat 713 (which extends through the substrate 702 and along the height of the microneedle body portion 712 as shown in FIG. 7A), the modified insulating moat 913 may extend only through the substrate 902 such that the sandwich structure filling the trench (e.g., created by DRIE as described above) forms only the buried feature in the substrate. Although the sidewall of the microneedle 900 is shown in FIG. 9 as extending generally orthogonal to the substrate surface, it should be understood that because the modified insulating moat 913 need not extend the entire height of the microneedle body portion 712, in some variations the sidewall of the microneedle 900 may be angled at non-orthogonal angles relative to the substrate (e.g., the sidewall may have a slight positive taper of between about 1 degree to about 10 degrees, or between about 5 degrees and about 10 degrees).

In some variations, the rest of the microneedle surface 900 (aside from the annular electrode 920) may include an insulating material extending from substrate insulation 904. For example, a layer of an insulating material (e.g., SiO₂) may extend from a frontside surface of the substrate 902 to provide a body portion insulation 918 and may further extend up over a proximal edge 921b of the electrode 920 as shown in FIG. 9. Another region of insulating material may similarly cover a distal edge 921a of the electrode 920 and insulate the distal apex 916. Such region of insulating material and/or modified insulating moat 913 may help prevent electrical contact between the conductive core 940 and the surrounding substrate 902. Accordingly, like the microneedle 700, the microneedle 900 may maintain electrical isolation for individual addressability within a microneedle array. In some variations, the process to form microneedle 900 may result in higher yield and/or provide lower production cost compared to the process to form microneedle 700.

The microneedle 900 may have any suitable dimensions. By way of illustration, the microneedle 900 may, in some variations, include a height of between about 400 μm and about 600 μm, or about 500 μm. In some variations, the tapered distal portion 914 may have a tip angle of between about 25 degrees and about 45 degrees, with a tip radius of less than about 100 nm. Furthermore, the microneedle may have a shaft diameter of between about 160 μm and about 200 μm.

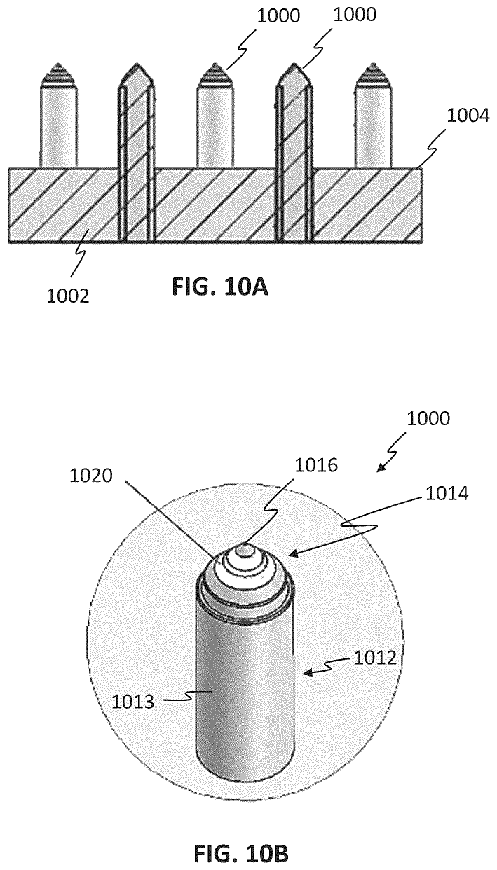

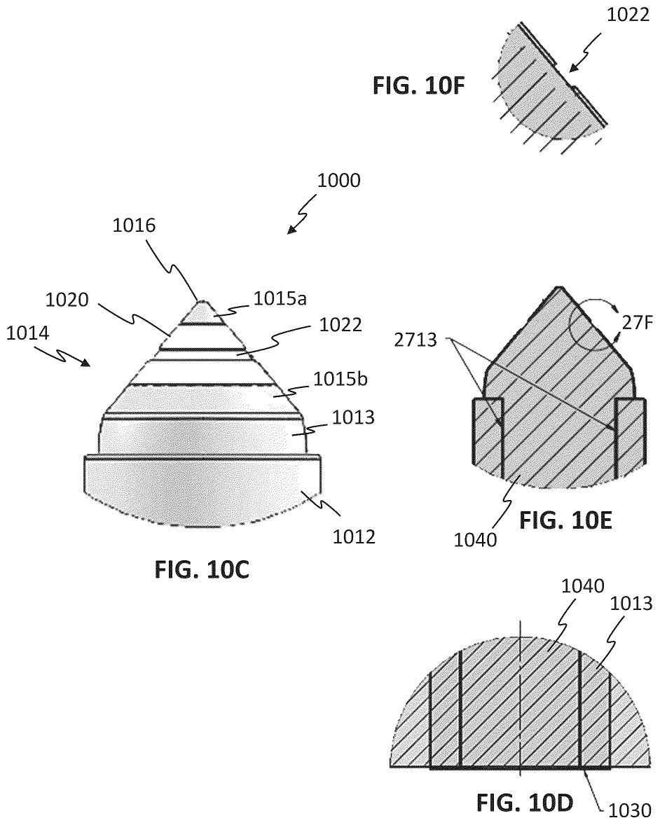

FIGS. 10A-10F illustrate another example variation of a microneedle 1000 having a generally columnar body portion extending from a substrate 1002 having a top surface 1004. The microneedle 1000 may be similar to microneedle 800 as described above, except as described below. For example, as shown in FIG. 10B, like the microneedle 800, the microneedle 1000 may include a columnar body portion 1012, and a tapered distal portion arranged on a cylinder 1013 and terminating in an insulated distal apex 1016. The cylinder 1013 may be insulated and have a smaller diameter than the columnar body portion 1012. The microneedle 1000 may further include an annular electrode 1020 that includes a conductive material and is arranged on the tapered distal portion at a location proximal to (or offset or spaced apart from) the distal apex 1016. Other elements of microneedle 1000 as shown in FIGS. 10A-10F have numbering similar to corresponding elements of microneedle 800.

As can most easily be seen in FIGS. 10B, 10C and 10F, the tapered distal portion 1014, and more specifically, the electrode 1020 on the tapered distal portion 1014 of the microneedle 1000, may include a tip contact trench 1022. This contact trench may be configured to establish ohmic contact between the electrode 1020 and the underlying conductive core 1040 of the microneedle. In some variations, the shape of the tip contact trench 1022 may include an annular recess formed in the surface of the tapered distal portion 1014. In some variations, the shape of the tip contact trench 1022 may include an annular recess formed in the surface of the conductive core 1040 (e.g., into the body portion of the microneedle, or otherwise in contact with a conductive pathway in the body portion). In some variations, the tip contact trench 1022 may be formed in the insulating material on the tapered distal portion 1014 and may have a depth about equal to the thickness of the insulating material (e.g., the distal insulating surface 1015a and/or the second distal insulating surface 1015b). In some instances, the depth of the contact trench may be greater than the thickness of the insulating material such that the contact trench extends beyond a surface of the conductive core 1040 (e.g., into the conductive core 1040). The electrode 1020 may overlie the tip contact trench 1022 such that ohmic contact is established between the electrode 1020 and the conductive core 1040. In some variations, the electrode 1020 may extend beyond the tip contact trench 1022 such that when the electrode 1020 material is deposited onto the conductive core 1040, the electrode 1020 with the tip contact trench 1022 may have a stepped profile when viewed from the side. The tip contact trench 1022 may thus advantageously help ensure contact between the electrode 1020 and the underlying conductive core 1040. Any of the other microneedle variations described herein may also have a similar tip contact trench to help ensure contact between the electrode (which may be, for example, a working electrode, reference electrode, counter electrode, etc.) with a conductive pathway within the microneedle.

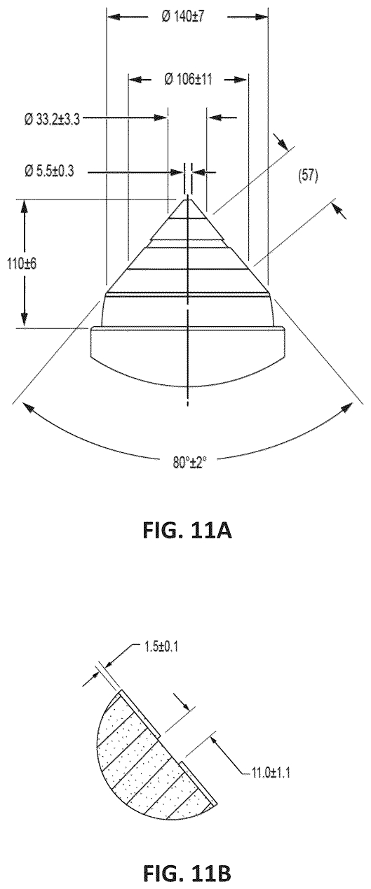

FIGS. 11A and 11B illustrate additional various dimensions of an example variation of a columnar microneedle with a tapered distal portion and annular electrode, similar to microneedle 1000 described above. For example, the variation of the microneedle shown in FIGS. 11A and 11B may have a tapered distal portion generally having a taper angle of about 80 degrees (or between about 78 degrees and about 82 degrees, or between about 75 degrees and about 85 degrees), and a cone diameter of about 140 μm (or between about 133 μm and about 147 μm, or between about 130 μm and about 150 μm). The cone of the tapered distal portion may be arranged on a cylinder such that the overall combined height of the cone and cylinder is about 110 μm (or between about 99 μm and about 116 μm, or between about 95 μm and about 120 μm). The annular electrode on the tapered distal portion may have an outer or base diameter of about 106 μm (or between about 95 μm and about 117 μm, or between about 90 μm and about 120 μm), and an inner diameter of about 33.2 μm (or between about 30 μm and about 36 μm, or between about 25 μm and about 40 μm). The length of the annular electrode, as measured along the slope of the tapered distal portion, may be about 57 μm (or between about 55 μm and about 65 μm), and the overall surface area of the electrode may be about 12,700 μm² (or between about 12,500 μm² and about 12,900 μm², or between about 12,000 μm² and about 13,000 μm²). As shown in FIG. 11B, the electrode may furthermore have a tip contact trench extending around a central region of the cone of the tapered distal portion, where the contact may have a width of about 11 μm (or between about 5 μm and about 50 μm, between about 10 μm and about 12 μm, or between about 8 μm and about 14 μm) as measured along the slope of the tapered distal portion, and a trench depth of about 1.5 μm (or between about 0.1 μm and about 5 μm, or between about 0.5 μm and about 1.5 μm, or between about 1.4 μm and about 1.6 μm, or between about 1 μm and about 2 μm). The microneedle has an insulated distal apex having a diameter of about 5.5 μm (or between about 5.3 μm and about 5.8 μm, or between about 5 μm and about 6 μm).

Details of example variations of microneedle array configurations are described in further detail below.

As described above, each microneedle in the microneedle array may include an electrode. In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode, at least one counter electrode, and at least one reference electrode. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of that electrode.

Generally, the working electrode is the electrode at which oxidation and/or reduction reaction of interest occurs for detection of an analyte of interest. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed potential relationship between the working electrode and reference electrode contingents within the electrochemical system (via an electronic feedback mechanism), while permitting the counter electrode to dynamically swing to potentials required to sustain the redox reaction of interest.

### Microneedle Array Configurations

Multiple microneedles (e.g., any of the microneedle variations described herein, each of which may have a working electrode, counter electrode, or reference electrode as described above) may be arranged in a microneedle array. Considerations of how to configure the microneedles include factors such as desired insertion force for penetrating skin with the microneedle array, optimization of electrode signal levels and other performance aspects, manufacturing costs and complexity, etc.

For example, the microneedle array may include multiple microneedles that are spaced apart at a predefined pitch (distance between the center of one microneedle to the center of its nearest neighboring microneedle). In some variations, the microneedles may be spaced apart with a sufficient pitch so as to distribute force (e.g., avoid a “bed of nails” effect) that is applied to the skin of the user to cause the microneedle array to penetrate the skin. As pitch increases, force required to insert the microneedle array tends to decrease and depth of penetration tends to increase. However, it has been found that pitch only begins to affect insertion force at low values (e.g., less than about 150 μm). Accordingly, in some variations the microneedles in a microneedle array may have a pitch of at least 200 μm, at least 300 μm, at least 400 μm, at least 500 μm, at least 600 μm, at least 700 μm, or at least 750 μm. For example, the pitch may be between about 200 μm and about 800 μm, between about 300 μm and about 700 μm, or between about 400 μm and about 600 μm. In some variations, the microneedles may be arranged in a periodic grid, and the pitch may be uniform in all directions and across all regions of the microneedle array. Alternatively, the pitch may be different as measured along different axes (e.g., X, Y directions) and/or some regions of the microneedle array may include a smaller pitch while other may include a larger pitch.

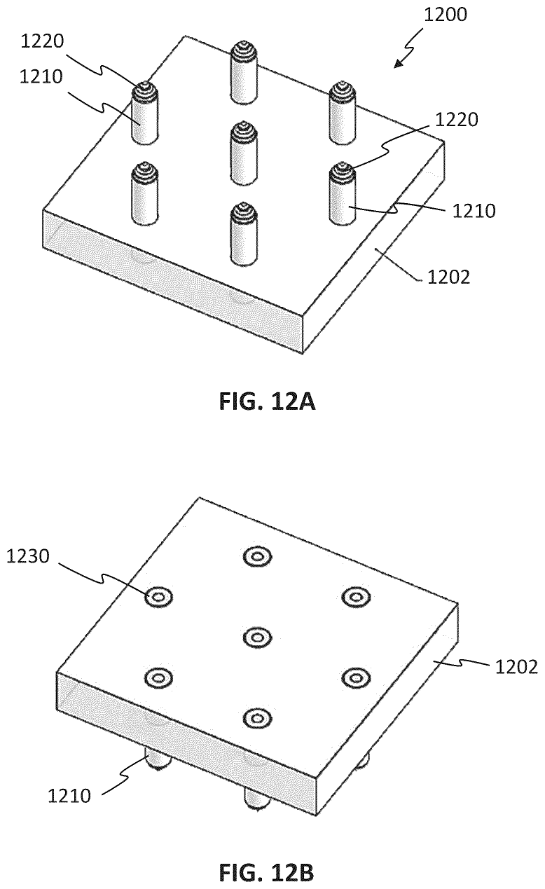

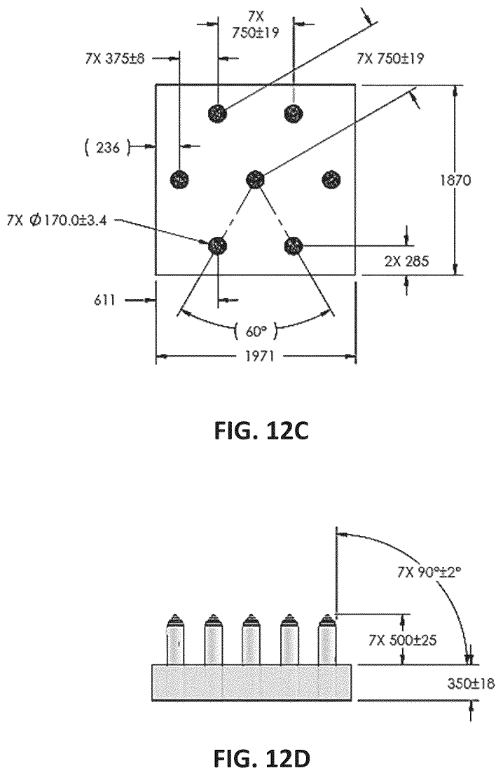

Furthermore, for more consistent penetration, microneedles may be spaced equidistant from one another (e.g., same pitch in all directions). To that end, in some variations, the microneedles in a microneedle array may be arranged in a hexagonal configuration as shown in FIGS. 12A-12C, 13A-13B, and 14A-14J. Alternatively, the microneedles in a microneedle array may arranged in a rectangular array (e.g., square array), or in another suitable symmetrical manner

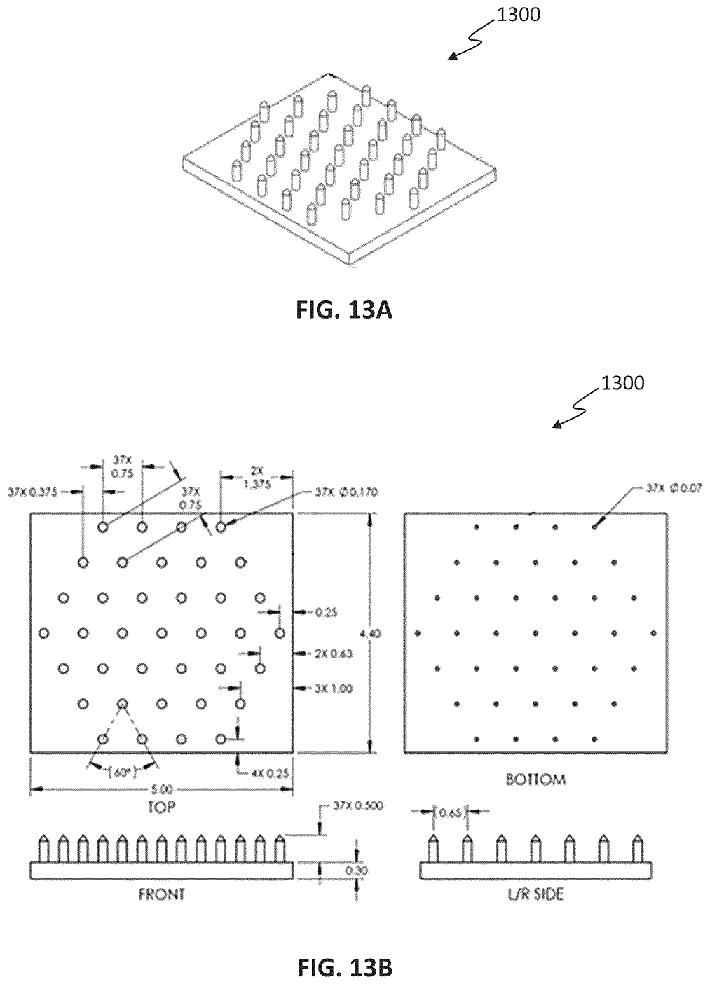

Another consideration for determining configuration of a microneedle array is overall signal level provided by the microneedles. Generally, signal level at each microneedle is invariant of the total number of microneedle elements in an array. However, signal levels can be enhanced by electrically interconnecting multiple microneedles together in an array. For example, an array with a large number of electrically connected microneedles is expected to produce a greater signal intensity (and hence increased accuracy) than one with fewer microneedles. However, a higher number of microneedles on a die will increase die cost (given a constant pitch) and will also require greater force and/or velocity to insert into skin. In contrast, a lower number of microneedles on a die may reduce die cost and enable insertion into the skin with reduced application force and/or velocity. Furthermore, in some variations a lower number of microneedles on a die may reduce the overall footprint area of the die, which may lead to less unwanted localized edema and/or erythema. Accordingly, in some variations, a balance among these factors may be achieved with a microneedle array including 37 microneedles as shown in FIGS. 13A-13B or a microneedle array including seven microneedles as shown in FIGS. 12A-12C. However, in other variations there may be fewer microneedles in an array (e.g., between about 5 and about 35, between about 5 and about 30, between about 5 and about 25, between about 5 and about 20, between about 5 and about 15, between about 5 and about 100, between about 10 and about 30, between about 15 and about 25, etc.) or more microneedles in an array (e.g., more than 37, more than 40, more than 45, etc.).

Additionally, as described in further detail below, in some variations only a subset of the microneedles in a microneedle array may be active during operation of the analyte monitoring device. For example, a portion of the microneedles in a microneedle array may be inactive (e.g., no signals read from electrodes of inactive microneedles). In some variations, a portion of the microneedles in a microneedle array may be activated at a certain time during operation and remain active for the remainder of the operating lifetime of the device. Furthermore, in some variations, a portion of the microneedles in a microneedle array may additionally or alternatively be deactivated at a certain time during operation and remain inactive for the remainder of the operating lifetime of the device.

In considering characteristics of a die for a microneedle array, die size is a function of the number of microneedles in the microneedle array and the pitch of the microneedles. Manufacturing costs are also a consideration, as a smaller die size will contribute to lower cost since the number of dies that can be formed from a single wafer of a given area will increase. Furthermore, a smaller die size will also be less susceptible to brittle fracture due to the relative fragility of the substrate.

Furthermore, in some variations, microneedles at the periphery of the microneedle array (e.g., near the edge or boundary of the die, near the edge or boundary of the housing, near the edge or boundary of an adhesive layer on the housing, along the outer border of the microneedle array, etc.) may be found to have better performance (e.g., sensitivity) due to better penetration compared to microneedles in the center of the microneedle array or die. Accordingly, in some variations, working electrodes may be arranged largely or entirely on microneedles located at the periphery of the microneedle array, to obtain more accurate and/or precise analyte measurements.

FIGS. 13A and 13B depict an illustrative schematic of 37 microneedles arranged in an example variation of a microneedle array 1300. The 37 microneedles may, for example, be arranged in a hexagonal array with an inter-needle center-to-center pitch of about 750 μm (or between about 700 μm and about 800 μm, or between about 725 μm and about 775 μm) between the center of each microneedle and the center of its immediate neighbor in any direction. FIG. 13A depicts an illustrative schematic of an example variation of a die including the microneedle arrangement. Example dimensions of the die (e.g., about 4.4 mm by about 5.0 mm) and the microneedle array 1300 are shown in FIG. 13B.

FIGS. 12A and 12B depict perspective views of an illustrative schematic of seven microneedles 1210 arranged in an example variation of a microneedle array 1200. The seven microneedles 1210 are arranged in a hexagonal array on a substrate 1202. As shown in FIG. 12A, the electrodes 1220 are arranged on distal portions of the microneedles 1210 extending from a first surface of the substrate 1202. As shown in FIG. 12B, proximal portions of the microneedles 1210 are conductively connected to respective backside electrical contacts 1230 on a second surface of the substrate 1202 opposite the first surface of the substrate 1202. FIGS. 12C and 12D depict plan and side views of an illustrative schematic of a microneedle array similar to microneedle array 1200. As shown in FIGS. 12C and 12D, the seven microneedles are arranged in a hexagonal array with an inter-needle center-to-center pitch of about 750 μm between the center of each microneedle and the center of its immediate neighbor in any direction. In other variations the inter-needle center-to-center pitch may be, for example, between about 700 μm and about 800 μm, or between about 725 μm and about 775 μm. The microneedles may have an approximate outer shaft diameter of about 170 μm (or between about 150 μm and about 190 μm, or between about 125 μm and about 200 μm) and a height of about 500 μm (or between about 475 μm and about 525 μm, or between about 450 μm and about 550 μm).

Furthermore, the microneedle arrays described herein may have a high degree of configurability concerning where the working electrode(s), counter electrode(s), and reference electrode(s) are located within the microneedle array. This configurability may be facilitated by the electronics system.

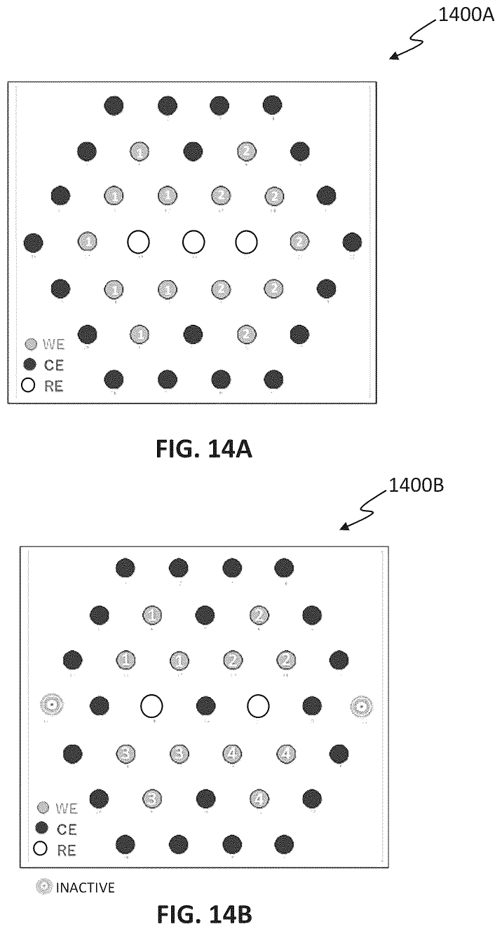

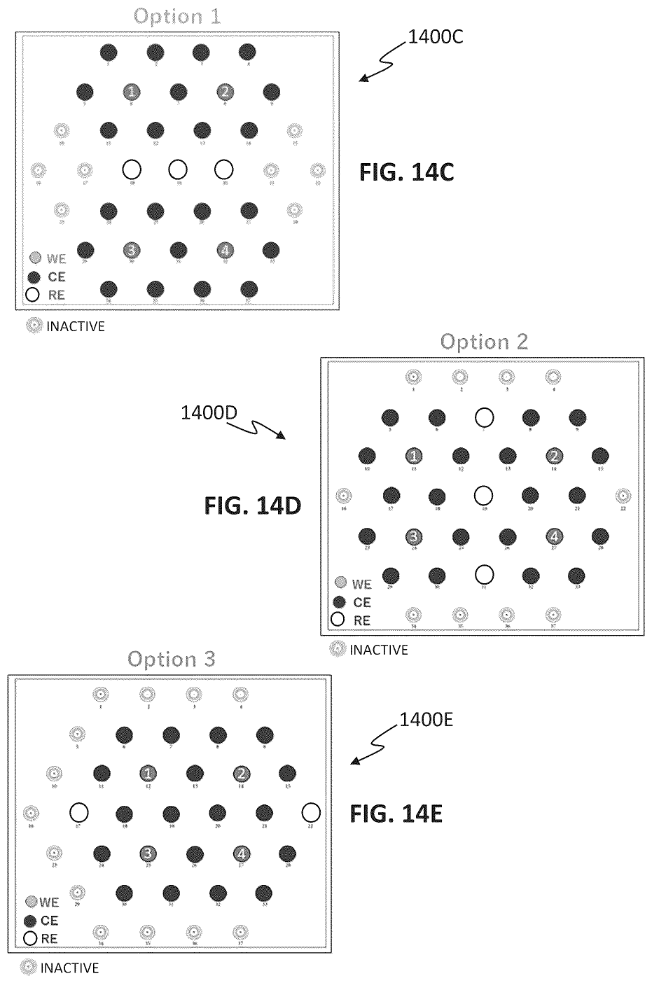

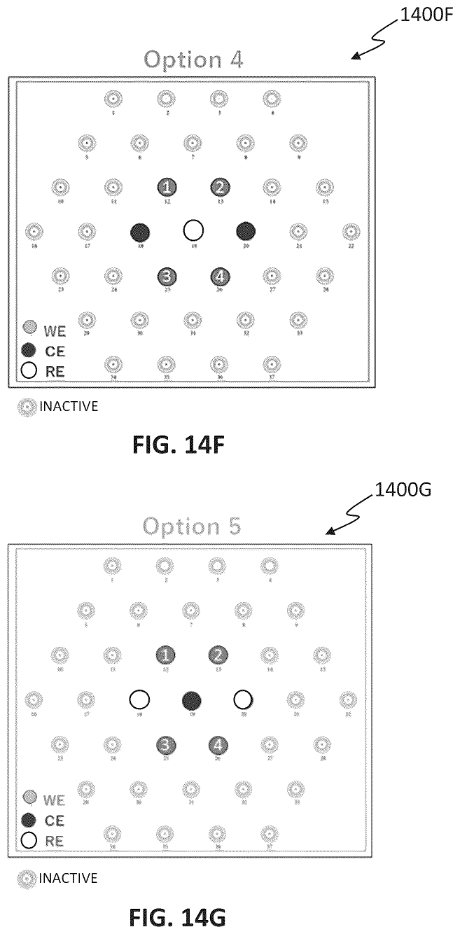

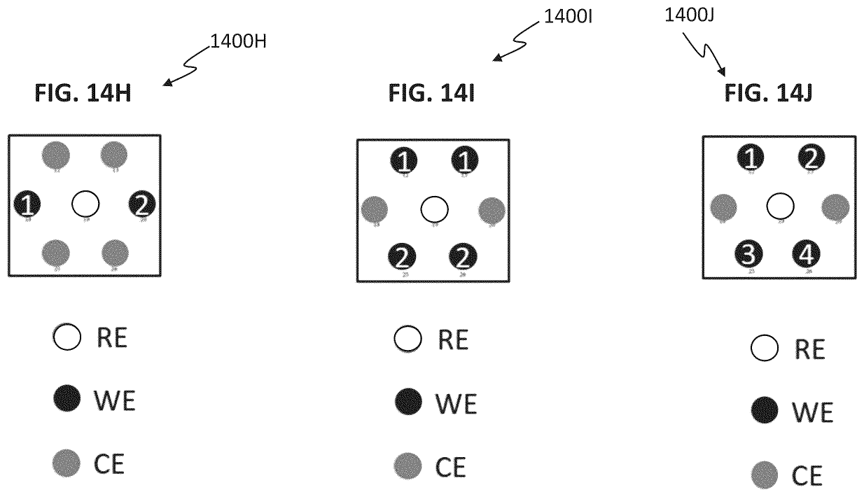

In some variations, a microneedle array may include electrodes distributed in two or more groups in a symmetrical or non-symmetrical manner in the microneedle array, with each group featuring the same or differing number of electrode constituents depending on requirements for signal sensitivity and/or redundancy. For example, electrodes of the same type (e.g., working electrodes) may be distributed in a bilaterally or radially symmetrical manner in the microneedle array. For example, FIG. 14A depicts a variation of a microneedle array 1400A including two symmetrical groups of seven working electrodes (WE), with the two working electrode groups labeled “1” and “2”. In this variation, the two working electrode groups are distributed in a bilaterally symmetrical manner within the microneedle array. The working electrodes are generally arranged between a central region of three reference electrodes (RE) and an outer perimeter region of twenty counter electrodes (CE). In some variations, each of the two working electrode groups may include seven working electrodes that are electrically connected amongst themselves (e.g., to enhance sensor signal). Alternatively, only a portion of one or both of the working electrode groups may include multiple electrodes that are electrically connected amongst themselves. As yet another alternative, the working electrode groups may include working electrodes that are standalone and not electrically connected to other working electrodes. Furthermore, in some variations the working electrode groups may be distributed in the microneedle array in a non-symmetrical or random configuration.

As another example, FIG. 14B depicts a variation of a microneedle array 1400B including four symmetrical groups of three working electrodes (WE), with the four working electrode groups labeled “1”, “2”, “3”, and “4.” In this variation, the four working electrode groups are distributed in a radially symmetrical manner in the microneedle array. Each working electrode group is adjacent to one of two reference electrode (RE) constituents in the microneedle array and arranged in a symmetrical manner. The microneedle array also includes counter electrodes (CE) arranged around the perimeter of the microneedle array, except for two electrodes on vertices of the hexagon that are inactive or may be used for other features or modes of operation.

In some variations, only a portion of microneedle array may include active electrodes. For example, FIG. 14C depicts a variation of a microneedle array 1400C with 37 microneedles and a reduced number of active electrodes, including four working electrodes (labeled “1”, “2”, “3”, and “4”) in a bilaterally symmetrical arrangement, twenty-two counter electrodes, and three reference electrodes. The remaining eight electrodes in the microneedle array are inactive. In the microneedle array shown in FIG. 19C, each of the working electrodes is surrounded by a group of counter electrodes. Two groups of such clusters of working electrodes and counter electrodes are separated by a row of the three reference electrodes.

As another example, FIG. 14D depicts a variation of a microneedle array 1400D with 37 microneedles and a reduced number of active electrodes, including four working electrodes (labeled “1”, “2”, “3”, and “4”) in a bilaterally symmetrical arrangement, twenty counter electrodes, and three reference electrodes, where the remaining ten electrodes in the microneedle array are inactive.

As another example, FIG. 14E depicts a variation of a microneedle array 1400E with 37 microneedles and a reduced number of active electrodes, including four working electrodes (labeled “1”, “2”, “3”, and “4”), eighteen counter electrodes, and two reference electrodes. The remaining thirteen electrodes in the microneedle array are inactive. The inactive electrodes are along a partial perimeter of the overall microneedle array, thereby reducing the effective size and shape of the active microneedle arrangement to a smaller hexagonal array. Within the active microneedle arrangement, the four working electrodes are generally in a radially symmetrical arrangement, and each of the working electrodes is surrounded by a group of counter electrodes.

FIG. 14F depicts another example variation of a microneedle array 1400F with 37 microneedles and a reduced number of active electrodes, including four working electrodes (labeled “1”, “2”, “3”, and “4”), two counter electrodes, and one reference electrode. The remaining thirty electrodes in the microneedle array are inactive. The inactive electrodes are arranged in two layers around the perimeter of the overall microneedle array, thereby reducing the effective size and shape of the active microneedle arrangement to a smaller hexagonal array centered around the reference electrode. Within the active microneedle arrangement, the four working electrodes are in a bilaterally symmetrical arrangement and the counter electrodes are equidistant from the central reference electrode.

FIG. 14G depicts another example variation of a microneedle array 1400G with 37 microneedles and a reduced number of active electrodes. The active electrodes in microneedle array 1400G are arranged in a similar manner as that in microneedle array 1400F shown in FIG. 14F, except that the microneedle array 1400G includes one counter electrode and two reference electrodes, and the smaller hexagonal array of active microneedles is centered around the counter electrode. Within the active microneedle arrangement, the four working electrodes are in a bilaterally symmetrical arrangement and the reference electrodes are equidistant from the central counter electrode.

FIG. 14H depicts another example variation of a microneedle array 1400H with seven microneedles. The microneedle arrangement contains two microneedles assigned as independent working electrodes (1 and 2), a counter electrode contingent comprised of 4 microneedles, and a single reference electrode. There is bilateral symmetry in the arrangement of working and counter electrodes, which are equidistant from the central reference electrode. Additionally, the working electrodes are arranged as far as possible from the center of the microneedle array (e.g., at the periphery of the die or array) to take advantage of a location where the working electrodes are expected to have greater sensitivity and overall performance.

FIG. 14I depicts another example variation of a microneedle array 1400I with seven microneedles. The microneedle arrangement contains four microneedles assigned as two independent groupings (1 and 2) of two working electrodes each, a counter electrode contingent comprised of 2 microneedles, and a single reference electrode. There is bilateral symmetry in the arrangement of working and counter electrodes, which are equidistant from the central reference electrode. Additionally, the working electrodes are arranged as far as possible from the center of the microneedle array (e.g., at the periphery of the die or array) to take advantage of a location where the working electrodes are expected to have greater sensitivity and overall performance.

FIG. 14J depicts another example variation of a microneedle array 1400J with seven microneedles. The microneedle arrangement contains four microneedles assigned as independent working electrodes (1, 2, 3, and 4), a counter electrode contingent comprised of 2 microneedles, and a single reference electrode. There is bilateral symmetry in the arrangement of working and counter electrodes, which are equidistant from the central reference electrode. Additionally, the working electrodes are arranged as far as possible from the center of the microneedle array (e.g., at the periphery of the die or array) to take advantage of a location where the working electrodes are expected to have greater sensitivity and overall performance.

While FIGS. 14A-14J illustrate example variations of microneedle array configurations, it should be understood that these figures are not limiting and other microneedle configurations (including different numbers and/or distributions of working electrodes, counter electrodes, and reference electrodes, and different numbers and/or distributions of active electrodes and inactive electrodes, etc.) may be suitable in other variations of microneedle arrays.

Additional details of example variations of microneedle array configurations are described in further detail below.

### Electrode(s)—Structure

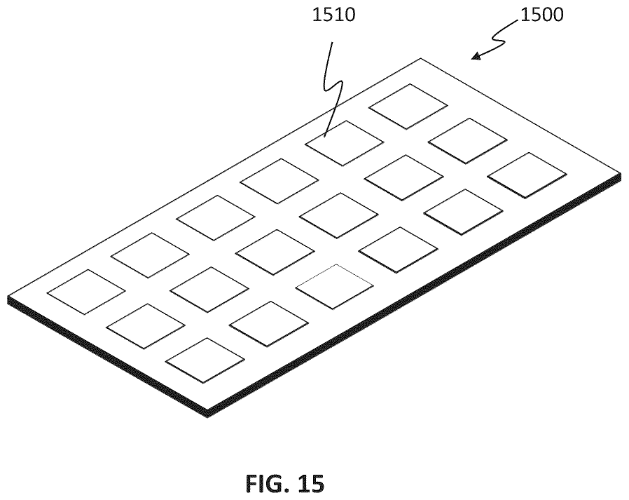

As described above, each microneedle in the microneedle array may include an electrode (by way of example electrode 520 of microneedle 510 as shown in FIG. 5B, electrode 720 of microneedle 710 as shown in FIGS. 7A and 7C, electrode 920 of microneedle 910 as shown in FIG. 9, electrode 1020 of microneedle 1010 as shown in FIGS. 10B and 10C, and electrode 1220 of microneedle 1210 as shown in FIG. 12A). In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode coupled to a first microneedle, at least one counter electrode coupled to a second microneedle, and at least one reference electrode coupled to a third microneedle. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of that electrode.

Whereas the electrodes (including working electrodes) have been primarily described herein above as being coupled to microneedles, it should be appreciated that the electrodes described herein, including working, counter, and reference electrodes, may be coupled to otherwise incorporated with other structures, such as a planar surface, a probe tip, an external or internal surface of a tubular structure, and the like. In some variations, one or more of the electrodes described herein may be planar electrodes, and may, in some instances, be planar microelectrodes (e.g., having a width or diameter less than 500 microns). FIG. 15 shows, by way of example, a microelectrode array 1500 comprising a plurality of planar microelectrodes 1510.

Generally, the working electrode is the electrode that selectively detects an analyte of interest through redox reactions with electrochemical methods. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed or controlled potential relationship between the working electrode and reference electrode contingents within the electrochemical system (via an electronic feedback mechanism), while permitting the counter electrode to dynamically swing to potentials required to sustain the redox reaction of interest.

Turning to the multi-thiol-based passivation approach of the E-AB sensors of the present disclosure, consistent with implementations of the current subject matter, aspects of a working electrode, a counter electrode, and a reference electrode are provided.

### Working Electrode

As described above, the working electrode is the electrode at which the detection of an analyte occurs. In some variations, the detection of the analyte may be performed at the interface of the working electrode and interstitial fluid located within the body (e.g., on an outer surface of the overall microneedle). In some variations, a working electrode may include an electrode material and a biorecognition layer in which a biorecognition element (e.g., an aptamer) is immobilized on the working electrode to facilitate selective analyte quantification. In some variations, the biorecognition layer may also function as an interference-blocking layer and may help prevent endogenous and/or exogenous species from directly oxidizing (or reducing) at the electrode. In some variations, the biorecognition layer may include a passivation element deposited on the electrode material. The passivation element prevents interfering electrochemical reactions at the electrode surface and minimizes capacitance. In some variations, the passivation element may comprise passivation molecules capable of being chemisorbed directly or indirectly onto the electrode material surface. In some variations, a passivation molecule may be a molecule that comprise, as a functional group, a thiol (which may be referred to herein as a “thiol-based passivation molecule”), for example a small molecule (“a thiol-bases small molecule”) or a peptide. In some variations, the passivation element may be a multi-component passivation element may comprise two or more types of thiol-based passivation molecules, which may be, for example, two or more types of thiol-based small molecules.

In some variations, the surface of the electrode is functionalized with a redox-active molecule via immobilization through an aptamer, and analyte binding to these surface sites follows the Michaelis-Menten model. Upon binding an analyte, an analyte-binding aptamer experiences a conformational change that moves the redox-active molecule closer, or further, from the electrode. The redox-active molecule is held in its oxidized state, and a sweep to more negative potential reduces the redox-active molecule within range of electron transfer. The final relationship is non-linear and is quasi-linear over a limited range when comparing the signal gain with the logarithm of analyte concentration. The relationship for redox current detected at the working electrode is represented by equation (3) below:

$$i = - nFA \cdot d Γ O / dt \qquad ( 3 )$$

where n is the stoichiometric number of electrons mitigating a redox reaction, F is Faraday's constant, A is electrode surface area, and dΓO/dt is the change in surface concentration of the oxidized form of the redox-active molecule with time.

Moreover, because the detected current is a direct function of electrode surface area A, the surface area of the electrode may be increased to enhance the sensitivity (e.g., amperes per molar of analyte) of the sensor. For example, multiple singular working electrodes may be grouped into arrays of two or more constituents to increase total effective sensing surface area. Additionally, or alternatively, to obtain redundancy, multiple working electrodes may be operated as parallelized sensors to obtain a plurality of independent measures of the concentration of an analyte of interest. The working electrode can either be operated as the anode (such that an analyte is oxidized at its surface) or as the cathode (such that an analyte is reduced at its surface).

i. Biorecognition Element

In some variations, the biorecognition element in the biorecognition layer may be a molecule that selectively binds to a given analyte. In some variations, the biorecognition element in the biorecognition layer may be a molecule that selectively and reversibly binds to a given analyte. In some instances, the biorecognition element may be an oligonucleotide. In some variations, the oligonucleotide may be DNA or RNA. The oligonucleotide may be functionalized at 3′ end or 5′ end. One end may provide a chemical moiety (“immobilization moiety”) for surface immobilization, such as an amine, aldehyde, carboxylic acid, thiol, disulfide, azide, n-hydroxysuccinimide (NHS), maleimide, vinyl, silane, chlorosilane, methoxysilane, ethoxysilane, or acetylene group. The immobilization moiety may be separated from the oligonucleotide sequence by a linker selected for its ability to create distance between the oligonucleotide sequence and the surface to which it is immobilized. The linker may also be chosen for its compatibility with other chemical layers on the electrode surface, for example, a hydrocarbon linker with equal or similar length to the hydrocarbon chain used in a self-assembled monolayer that is coating the remainder of the electrode surface. The opposite end of the oligonucleotide may be functionalized with one or more redox active molecules, by way of example methylene blue, ferrocene, pentamethyl ferrocene, C5-ferrocene, Nile blue, thionine, anthraquinone, hydroquinone, C5-anthraquinone, gallocyanine, indophenol, neutral red, dabcyl, π extended tetrathiafulvalene (exTTF), or carboxy-X-rhodamine, that serve as a probe. These redox-active molecules may also be attached to the oligonucleotide through a custom linker. The backbone of the oligonucleotide may be modified to increase stability in physiological conditions. For example, an RNA sequence incorporating L-ribose or a DNA sequence incorporating L-deoxyribose, as opposed to their natural respective dextrorotary sugars, may be used to protect the oligonucleotide from degradation by enzymes in the body. In some variations, a backbone modification may include replacing ribose in RNA or deoxyribose in DNA with 2′-O-methyl ribose, also with the effect of protection from enzyme cleavage in physiological conditions.

In some variations, the oligonucleotide may comprise a region having a nucleotide sequence complementary to a given nucleic acid analyte, by way of example a viral or bacterial gene or regulatory region. In some variations, the oligonucleotide may be an aptamer.

In some instances, the biorecognition element may be a peptide. The peptide may be an antibody or a portion thereof, such as a nanobody (also known as an VHH antibody) that comprises an antigen binding fragment of heavy chain only antibodies, that selectively binds a given analyte.

In some instances, the molecule may be an aptamer (an “analyte-binding aptamer”). An aptamer is a peptide or single-stranded oligonucleotide that folds into a defined structure that selectively binds to a specific analyte (which may be referred to as target), which may be, by way of example, a protein, a peptide, a hormone, a nucleic acid, or a small molecule. Recognition and binding of an aptamer to its target involve three-dimensional, shape-dependent interactions as well as hydrophobic interactions, base-stacking, and intercalation, and are typically reversible through dissociation. Aptamers with affinity for a desired target are conventionally selected from a large oligonucleotide library through a process called SELEX (Systematic Evolution of Ligands by Exponential Enrichment). Through an iterative process, non-binding aptamers are discarded and aptamers binding to the proposed target are amplified by polymerase chain reaction (PCR). The iterative process may include counter-selection (using interferents and structurally similar molecules) to discard aptamers with insufficient selectivity toward analytes. Multiple rounds of SELEX may be performed with increasing stringency to enhance enrichment of the oligonucleotide pool, until one or more oligonucleotides having a desired degree of affinity and selectivity for the desired target are selected for use.

In some variations, the analyte-binding aptamer is a cortisol-binding aptamer defined by the following DNA sequence, 5′-GGACGACGCCAGAAGTTTACGAGGATATGGTAACATAGTCGT-3′ (SEQ ID NO: 1), where G, A, C, and T represent the typical DNA nucleotides containing guanine, adenine, cytosine, and thymine, respectively.

In some variations, the analyte-binding aptamer may be selected not for maximal affinity for analyte, but for an intermediate degree of affinity such that the portion of a population of the selected aptamer having an analyte molecule bound to it is sensitive to a physiological concentration range of analyte within dermal interstitial fluid, which may be between about 1 pmol/L and about 10 mmol/L or between about 0.001 μmol/L and about 1 μmol/L. In some variations, selection criteria of the analyte-binding aptamer may include the analyte-binding aptamer having between about 10% and about 75% “on” gain from minimum to maximum analyte concentrations and/or having between about 10% to about 40% “off” gain from minimum to maximum analyte concentrations. A signal “on gain” may refer to a set of square wave voltammetry parameters (frequency, peak value, step height) selected towards maximizing a current signal obtained in the presence of a target analyte. A signal “off gain” may refer to a set of square wave voltammetry parameters selected towards minimizing the current signal obtained in the presence of a target analyte. Sensitivity of the aptamer to analyte in dermal interstitial fluid advantageously allows for avoiding interference or signal degradation over time from biofouling or irreversible changes to the aptamer structure due to folding or damage.

The analyte-sensing aptamer may be functionalized with a redox-active molecule, by way of example methylene blue, ferrocene, pentamethyl ferrocene, C5-ferrocene, Nile blue, thionine, anthraquinone, C5-anthraquinone, hydroquinone, gallocyanine, indophenol, neutral red, dabcyl, exTTF, or carboxy-X-rhodamine, Where the aptamer is an oligonucleotide, the redox-active molecule may be functionalized at the 3′ end or 5′ end of the aptamer. A specific and reversible binding of analyte to the analyte-binding aptamer and the resultant conformational change of the analyte-binding aptamer may leads to a change in the proximity, and thus electron transfer characteristics, between the redox-active molecule and the working electrode to which the aptamer is bound. that is corresponding to the analyte concentration. Due to the analyte-binding property of the aptamer, the change in the electron transfer characteristics of the electrode corresponds to analyte concentration, and the electron transfer characteristics may be interrogated by various electrochemical techniques such as voltammetry, potentiometry, chronoamperometry, and/or electrochemical impedance spectroscopy. Voltammetry techniques vary the potential as a function of time and the resulting current is plotted as a function of potential. For example, cyclic voltammetry (CV) sweeps the potential of the cell linearly across a voltage range, while a fast scan CV (FSCV) technique does this at a faster rate. Alternating current voltammetry (ACV) uses application of a sinusoidally oscillating voltage to an electrochemical cell. Square wave voltammetry (SWV) uses a square wave superimposed over a staircase function to provide a sweeping measurement that provides two sampling instances per potential. As a result of this sampling technique, the contribution to the total current that results from non-faradic currents is minimized in SWV. In potentiometry, an open circuit potential is measured between a reference electrode and a working electrode. In chronoamperometry, the potential is stepped at the beginning of a measurement and then remains constant throughout the duration of the measurement, and the current that results from this stimulus may be plotted as a function of time. In electrochemical impedance spectroscopy, the complex impedance of the electrode is determined at one or more frequencies. Contributions to impedance (or admittance) from resistive and reactive circuit elements may be dependent on the position of redox probes tethered to surface-bound aptamers and correlate with analyte concentration.

ii. Passivation Element

In some variations, the devices described herein may include a passivation element to assist in reducing or preventing interference within the biorecognition layer and/or at the surface of the electrode material. More specifically, in some instances, functionalization of an available surface of an electrode material with a biorecognition element only may result in biorecognition element molecules that are too close to one another and may thus interfere with one another's ability to function as intended. In these instances, it may be disadvantageous or non-optimal to have all available surfaces of an electrode material occupied by biorecognition element molecules. On the other hand, the presence of exposed electrode material surfaces can cause an unwanted increase in electrochemical interactions unrelated to the binding of the biorecognition element with analytes, resulting in increased noise in the signal generated by the electrode. Accordingly, in some variations of the sensors described herein, the biorecognition layer may further comprise, with the biorecognition element, a passivation element acting to shield the exposed electrode material surfaces from unwanted chemical reactions. The passivation element may comprise a plurality of passivation molecules capable of being chemisorbed directly or indirectly onto the electrode material surface. In some variations, the passivation molecules may be thiol-group comprising molecules (“thiol-based passivation molecules”), for example small molecules or peptides. In some variations, the number of plurality of passivation molecules is based on a density of passivation molecules on the surface of the electrode. For instance, the density may be between about 200 molecules per nm² and about 1000 molecules per nm², between about 300 molecules per nm² and about 900 molecules per nm², between about 400 molecules per nm² and about 800 molecules per nm², and/or between about 500 molecules per nm² and about 700 molecules per nm². In some variations, the passivation molecules may form a monolayer on the electrode material surface. In some variations, the passivation molecules may form a bi-layer or other multi-layered (e.g., three, four, five or more) structure on the electrode material surface. The passivation element may function to prevent or reduce direct interaction between the electrode material surface and molecules dissolved within a biological fluid, without significantly interfering with the function of the biorecognition element. In some variations, the passivation element described herein may also act to provide or improve biocompatibility as well as provide anti-fouling properties of the working electrode. In some embodiments, the passivation element may also serve to provide a desired surface density of biorecognition elements (e.g., aptamers) interspersed amid passivation molecules on the surface of the electrode material.

In some variations, the plurality of passivation molecules of the passivation element may comprise a thiol-group-comprising small molecule (which may be referred to interchangeably herein as “small-molecule thiol”). In some variations, the plurality of passivation molecules may comprise one, two, three, four, five, six, seven, eight, or more different types of small-molecule thiols. A passivation element comprising two or more types of passivation molecules may be referred to as a “multi-component passivation element”, Each small-molecule thiol may be tethered to the electrode material and may also act to passivate the exposed electrode material (e.g., gold) surface from unwanted chemical reactions, such as the reduction of oxygen. In some variations, each small-molecule thiol may have the following structure:

$$HS - CX - Z \qquad ( formula 1 )$$

where X has a value of 2 to 16, which composes a linear, non-branching hydrocarbon chain, and where Z is a terminal functional group selected from the groups consisting of: hydrogen, hydroxyl, carboxyl, amine, trimethyl ammonium, phosphatidylcholine, sulfonate, sulfate ester, phosphonate, phosphate ester, an oligomer of polyethylene glycol (PEG) 1 to 50 units long (also known as PEG/polyethylene oxide (PEO)). For example, the small-molecule thiol may comprise a hydrophilic moiety on the end opposite from the thiol group tethered to the electrode material. In some variations, the small-molecule thiol may be one selected from the group consisting of: 1-hexanethiol, 6-mercapto-1-hexanamine, 6-mercapto-1-phosphatidylcholine hexane, 6-mercapto-1-hexanol (MCH), 7-mercapto-1-heptanol, 8-mercapto-1-octanol (MCO), 9-mercapto-1-nonanol, 10-mercapto-1-decanol, 11-mercapto-1-undecanol, 6-amino-1-hexanethiol, 7-amino-1-heptanethiol, 8-amino-1-octanethiol, 9-amino-1-nonanethiol, 10-amino-1-decanethiol, 11-amino-1-undecanethiol, (6-mercaptohexyl)-N,N,N-trimethylammonium bromide, (7-mercaptoheptyl)-N,N,N-trimethylammonium bromide, (8-mercaptooctyl)-N,N,N-trimethylammonium bromide, (9-mercaptononyl)-N,N,N-trimethylammonium bromide, (10-mercaptodecyl)-N,N,N-trimethylammonium bromide, (11-mercaptoundecyl)-N,N,N-trimethylammonium bromide, 6-mercaptohexylphosphoric acid, 7-mercaptoheptylphosphoric acid, 8-mercaptooctylphosphoric acid, 9-mercaptononylphosphoric acid, 10-mercaptodecylphosphoric acid, 11-mercaptoundecylphosphoric acid, 6-mercaptohexanoic acid, 7-mercaptoheptanoic acid, 8-mercaptooctanoic acid, 9-mercaptononanoic acid, 10-mercaptodecanoic acid, 11-mercaptoundecanoic acid, 6-mercaptohexanesulfonate, 7-mercaptoheptanesulfonate, 8-mercaptooctanesulfonate, 9-mercaptononanesulfonate, 10-mercaptodecanesulfonate, 11-mercaptoundecanesulfonate, poly(ethylene glycol) dithiol, 1,6-hexanedithiol, 1,7-heptanedithiol, 1,8-octanedithiol, 1,9-nonanedithiol, 1,10-decanedithiol, 1,11-undecanedithiol, 2-((3-((6-mercaptohexyl)thio)-2-methylpropanoyl)oxy)ethyl (2-trimethylammonium)ethyl)phosphate (PC), thiolated oligoethylene glycol (OEG), thiolated polyethylene glycol, (3-((6-mercaptohexyl)thio)-2-methylpropanoate-(2-ethyltrimethylammonium) chloride (AC), (3-((6-mercaptohexyl)thio)-2-methylpropanoate-(2-ethylsulfonate) potassium (SP), and (3-((6-mercaptohexyl)thio)-2-methylpropanoate-(2-ethyldimethylammonio-(3-propanesulfonate)) (AP), 1,2-bis-(11-sulfanyloundecanoyl)-sn-glycero-3-phosphocholine, methacrylate alkyl thiol ester, methacrylate polyethylene glycol (PEG) thiol ester, acrylate alkyl thiol ester, acrylate PEG thiol ester, vinyl terminated alkyl thiol, vinyl terminated PEG thiol, acetylene terminated alkyl thiol, acetylene terminated PEG thiol, benzophenone terminated alkyl thiol, benzophenone terminated PEG thiol, azide terminated alkyl thiol, azide terminated PEG thiol, N-hydroxysuccinimide terminated alkyl thiol, N-hydroxysuccinimide terminated PEG thiol, ferrocene terminated alkyl thiol, ferrocene terminated PEG thiol, methylene blue terminated alkyl thiol, methylene blue terminated PEG thiol, anthraquinone terminated alkyl thiol, anthraquinone terminated PEG thiol, hydroquinone terminated alkyl thiol, hydroquinone terminated PEG thiol, RGD peptide terminated thiol, YIGSR peptide terminated thiol, and the entire homologous series of any of the previously mentioned thiols with different carbon chain lengths.

In some variations, in the case of multi-component passivation elements, the plurality of passivation molecules of the passivation element may comprise a plurality of a first small-molecule thiol, or first thiol-based passivation molecule, and a plurality of a second, different small-molecule thiol, or second, different thiol-based passivation molecule. Each first thiol-based passivation molecule and each second, different thiol-based passivation molecule may be selected from the above-described listing of small-molecule thiols. For instance, the plurality of the first thiol-based passivation molecule may be MCH and the plurality of the second, different thiol-based passivation molecule may be PC. In some variations, the passivation element may further comprise a plurality of a third, different thiol-based passivation molecule, a plurality of a fourth, different thiol-based passivation molecule, a plurality of a fifth, different thiol-based passivation molecule, and/or a plurality of a sixth, different thiol-based passivation molecule. For instance, the first thiol-based passivation molecule may be MCH, the second, different thiol-based passivation molecule may be PC, and the third, different thiol-based passivation molecule may be ferrocene terminated alkyl thiol.

In some variations, the inclusion of a multi-component passivation element (e.g., comprising two or more small-molecule thiols). extends sensor lifetime. For instance, sensor lifetime of the sensors described herein may be at least 7 days, at least 8 days, at least 9 days, at least 10 days, at least 11 days, at least 12 days, at least 13 days, and/or at least 14 days. Similarly, sensor half-life, a measure of sensor lifetime, may be at least 3 days, at least 4 days, at least 5 days, at least 6 days, and/or at least 7 days.

In some variations, a distal end of each of the plurality of passivation molecules may comprise one or more of a hydrophilic moiety, a hydrophobic moiety, a charged moiety, and a zwitterionic moiety. For instance, the distal end of at least one of the plurality of passivation molecules may comprise a zwitterionic moiety. In some variations, the zwitterionic moiety may be a zwitterionic phosphorylcholine head group.

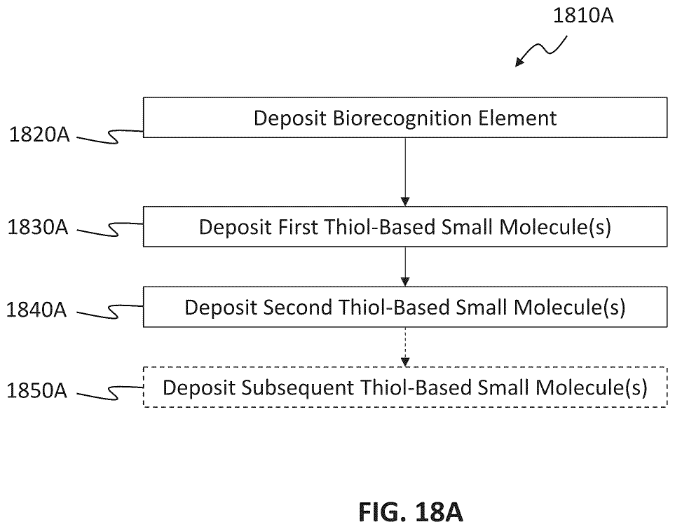

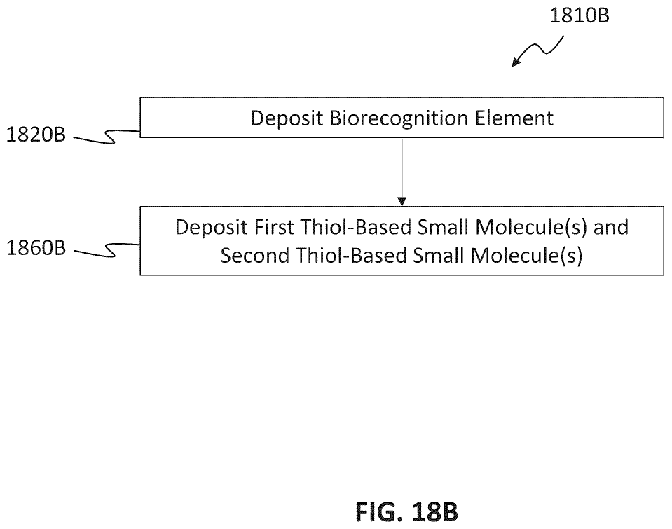

In some variations, such as, for example, upon deposition of the passivation element according to methods described herein with reference to FIG. 18A and FIG. 18B, a pseudo-film may form on the electrode material surface. Though a thickness of the pseudo-film may be based in part on whether a monolayer or a multilayer is formed, and thus may be dependent on a natural length of the small-molecule thiols, the thickness may also be predetermined and controllable during deposition. Regardless, the thickness of the pseudo-film formed by the passivation element may be between about 0.001 nm and about 1000 nm, between about 0.01 nm and about 100 nm, between about 0.1 nm and about 10 nm, between about 0.05 nm and about 500 nm, between about 0.1 nm and about 200 nm, between about 1 nm and about 1000 nm, between about 1 μm and about 500 μm, between about 2 μm and about 400 μm, between about 3 μm and about 300 μm, between about 4 μm and about 200 μm, between about 5 μm and about 100 μm, between about 10 μm and about 50 μm, or between about 20 μm and about 40 μm. For instance, the thickness of the pseudo-film formed by the passivation element may be between about 0.1 nm and about 10 nm.

Without being bound by any particular theory, the anti-fouling properties provided by utilizing a passivation element on the surface of the electrode material may be improved by configuring the electrode material to have a low surface net charge, e.g., a surface net charge of zero. When used in biosensors, self-assembled monolayers with net positive or net negative charges are susceptible to biofouling due to non-specific adsorption of proteins such as fibrinogen and lysozymes. One possible approach to reduce biofouling, as described herein, may be to use a zwitterionic passivation element, as introduced above. Accordingly, in some variations, the plurality of passivation molecules of the passivation element may comprise zwitterions. A zwitterion, which may be referred to in the art as an “inner salt” or “dipolar ion”, is a molecule that contains a mixture of positively charged (cationic) and negatively charged (anionic) functional groups. In some variations, the zwitterion may have an equal number of positively- and negatively-charged functional groups, so that the net charge of the zwitterion is neutral (that is, the positively charged and negatively charged functional groups balance each other such that the molecule has a net charge of zero). A zwitterion is typically characterized with an isoelectric point, often represented as pI, pH (I), or IEP, which is the pH value at which the net charge of the molecule is zero. In some variations, the passivation element may be or may comprise a zwitterion.

A zwitterion may in some cases be a peptide, and in those cases may be referred to as a “zwitterionic peptide”. In some variations, the plurality of passivation molecules of the passivation element may comprise a zwitterionic peptide. In some variations, the passivation element may comprise a mixture of one or more types of small molecule thiols and one or more types of zwitterionic peptides. In some variations, the passivation element may consist of one or more types of zwitterionic peptides. The zwitterionic nature of a peptide may be based on the presence of a plurality of charged functional groups, which may be negatively charged (anionic) and positively charged (cationic). One or more of the charged functional groups may be an R-group (side chain) of a given peptide's constituent amino acids. Certain amino acids have a side chain that is positively charged (cationic) or negatively charged (anionic). By way of example, a glutamate or aspartate residue comprises a carboxyl group as a side chain that is negatively charged, and a lysine residue comprises an anime group as a side chain that is positively charged. Moreover, the N-terminus of a peptide, unless modified to be neutral in charge, is a free amine group that is a positively charged functional group, and the C-terminus of a peptide, unless modified to be neutral in charge, is a free carboxyl group that is a negatively charged functional group. A zwitterionic peptide as used herein refers to a peptide of three or more amino acids that comprises at least one negatively charged functional group and at least one positively charged functional group. In some variations, the zwitterionic peptide comprises at least one amino acid with a positively charged side chain, at least one amino acid with a negatively charged side chain, or a combination thereof. In some variations, the zwitterionic peptide may have a length of 3, 4, 5, 6, 7, 8, 9, 10, 11, 12, 13, 14, 15, 16, 17, 18, 19 or 20 amino acids. In some variations, the N-terminus may be an unmodified free amine group or a modified N-terminus that is neutral in charge. In some variations, the C-terminus may be an unmodified free carboxyl group or a modified C-terminus that is neutral in charge. For example, for the C-terminus of the zwitterionic peptide to be neutral, the C-terminus may be modified to be an amide group, an ester, a methoxy ester, or a methoxide group. For the N-terminus of the zwitterionic peptide to be neutral, it may be modified to be an amide (e.g., an acetamide through an N-terminal acetyl modification) or an alkylamine (through an N-terminal alkylamine modification). In some variations, the number of positive functional groups may exceed the number of negative functional groups by not more than two, or not more than one. In some variations, the zwitterionic peptide may have an equal number of positively- and negatively-charged functional groups, so that the net charge of the zwitterionic peptide is neutral. The charge of a functional group, side chain, or peptide is dependent on the pH of the surrounding environment. As such, functional groups, side chains, or peptides are described herein as having positive, negative, or neutral charge with respect to when the peptide is in a surrounding environment that is at physiologic pH (approximately pH 7.4). Using a zwitterionic peptide as a component of the passivation element in a sensor as described herein may provide several advantages. These advantages include but are not limited to: (1) improved hydrophilicity of the electrode surface; (2) better blocking of the non-specific adsorption of proteins on the electrode surface in vivo; (3) reduced need of biologically incompatible reagents in the production process, (4) non-odorous, thereby removing the need for a fume hood during the production process; and (5) lower toxicity of an electrode functionalized therewith, when applied to or inserted within a subject.

In some variations, the amino acids of the zwitterionic peptide may be selected, and the N-terminus, the C-terminus, or both may be optionally modified to have a neutral charge as appropriate, in order for the net charge of the peptide to be neutral In some variations of the zwitterionic peptide, the count of amino acids with a negatively charged R-group may equal the count of amino acids with a positively charged R-group, so that the zwitterionic peptide has a net neutral charge. In some variations, the count of amino acids with a negatively charged R-group may exceed by one the count of amino acids with a positively charged R-group, the N-terminus may be a positively charged free amine group, and the C-terminus (which in an unmodified state is a negatively charged free carboxyl group) may be a modified C-terminus that is a neutral functional group (i.e., an amide group), so that the zwitterionic peptide has a net neutral charge. In some variations, the count of amino acids with a positively charged R-group may exceed by one the count of amino acids with a negatively charged R-group, the C-terminus may be a negatively charged free carboxyl group, and the N-terminus (which in an unmodified state is a positively charged amine group) may be a modified such that the N-terminus may have a neutral functional group, such as amide group, so that the zwitterionic peptide has a net neutral charge.

In some variations, the at least one negatively charged R-group may be a carboxyl group. The carboxyl group may be the side chain of a glutamate residue or an aspartate residue. In some variations, the at least one negatively charged functional group may be located at the N-terminal amino acid of the peptide. In some variations, the at least one R-group having the negative charged charge may be comprised in an N-terminal glutamate residue or an N-terminal aspartate residue.

In some variations, the at least one positively charged (cationic) functional group may be one of or a combination of two or more of an amine group, a guanidine group, and an imidazole group. The amine group may be the side chain of a lysine residue, or the side chain of an ornithine residue. The guanidine group may be the side chain of an arginine residue. The imidazole group may be the side chain of a histidine residue.

In some variations, the zwitterionic peptide may comprise at least one thiol group, which may be, or may be capable of being, chemisorbed to the surface of the electrode material. As such, the at least one thiol group may form, or may be capable or forming, a thiol linker tethering the zwitterionic peptide to the electrode material. The thiol group may be the side chain of a cysteine residue. In some variations, the cysteine residue may be a terminal cysteine, that is located at the C-terminal position or the N-terminal position of the zwitterionic peptide. In some variations, the cysteine residue may be a chemically modified cysteine residue, e.g., with an extended carbon chain of between 1 and 20 additional carbon atoms comprised in its thiol group, e.g., between the cysteine residue's central alpha carbon atom and the sulfur atom of the thiol group. In some variations, N-terminus or the C-terminus of the terminal cysteine residue may be modified to maintain charge neutrality. Without being bound by theory, the terminal cysteine having a negatively charged free amine at the N-terminus or a positively charged free carboxyl group may, in at least some instances, interfere with the chemisorption of the thiol group to the electrode material. As such, it may be advantageous to make neutral whichever terminus (N-terminus or C-terminus) the terminal cysteine is located so that it does not detract from the peptides' ability to be chemisorbed onto the electrode material. By way of example, where the terminal cysteine is a C-terminal cysteine, the C-terminus of a C-terminal cysteine may be modified to be an amide group, an ester, a methoxy ester, or a methoxide group. By way of another example, where the terminal cysteine is an N-terminal cysteine, the N-terminus of an N-terminal cysteine may be modified to be an amide (e.g., an acetamide) or an alkylamine.

In some variations, the zwitterionic peptide may comprise at least one non-canonical amino acid. For example, the at least one non-canonical amino acid may be ornithine, Beta-alanine, γ-aminobutyric acid (GABA), 4-aminobenzoic acid, taurine, or a combination thereof.

In some variations, the zwitterionic peptide may comprise or may consist of an amino acid sequence (N-terminal to C-terminal) of A-(BA)ₓ-P_{y}C or CP_{y}-(BA)ₓ-B, where A is an amino acid comprising a negatively charged side chain, B is an amino acid comprising a positively charged side chain, P is a proline residue, and C is a cysteine residue. In some variations, A and/or B may be one of the 20 canonical amino acids, a chemically modified canonical amino acid, or a non-canonical amino acid. In some variations, A may be a glutamic acid residue or an aspartic acid residue. In some variations, B may be a lysine residue, an arginine residue, a histidine residue, or an ornithine residue. In some variations, one or more amino acids may be chemically modified. In some variations, the proline residue may be a chemically modified proline residue. In some variations, the cysteine residue may be a chemically modified cysteine residue, e.g., with an extended carbon chain of between 1 and 20 additional carbon atoms comprised in its thiol group, e.g., between the cysteine residue's central alpha carbon atom and the sulfur atom of the thiol group. In some variations, a C-terminus of a C-terminal cysteine in the case of sequence A-(BA)ₓ-P_{y}C or an N-terminus of an N-terminal cysteine in the case of sequence CP_{y}-(BA)ₓ-B may be modified to have a neutral functional group. For example, the C-terminus of the C-terminal cysteine may be an amide group (through amidation), or the N-terminus of the N-terminal cysteine may be an acetamide group (through acetylation). In some variations, x may be between 0 and 5, between 0 and 2, between 0 and 3, between 0 and 4, between 1 and 5, between 1 and 3, between 1 and 4, or between 1 or 2, or may be 0, 1, 2, 3, 4, or 5. In some variations, y may be between 0 and 5, between 1 and 5, between 1 and 3 or may be 0, 1, 2, 3, 4, or 5.

In some variations, the zwitterionic peptide may comprise or may consist of an amino acid sequence of (N-terminal to C-terminal) X-(KX)ₓ-P_{y}C or CP_{y}-(KX)ₓ-K, where X is a glutamic acid residue or an aspartic acid residue, K is a lysine residue, P is a proline residue, and C is a cysteine residue. In some variations, the zwitterionic peptide may comprise or may consist of an amino acid sequence of (N-terminal to C-terminal) E-(KE)ₓ-P_{y}C or CP_{y}-(KE)ₓ-K, where E is a glutamic acid residue, K is a lysine residue, P is a proline residue, and C is a cysteine residue. In some variations, one or more amino acids may be chemically modified. In some variations, the proline residue may be a chemically modified proline residue. In some variations, the cysteine residue may be a chemically modified cysteine residue, e.g., with an extended carbon chain of between 1 and 20 additional carbon atoms comprised in its thiol group, e.g., between the cysteine residue's central alpha carbon atom and the sulfur atom of the thiol group. In some variations, the C-terminus of a C-terminal cysteine in the case of sequence X-(KX)ₓ-P_{y}C or E-(KE)ₓ-P_{y}C, or an N-terminus of an N-terminal cysteine in the case of sequence CP_{y}-(KX)ₓ-K or CP_{y}-(KX)ₓ-K, may be modified to have a neutral functional group. For example, the C-terminus of the C-terminal cysteine may be an amide group (through amidation), or the N-terminus of the N-terminal cysteine may be an acetamide group (through acetylation). In some variations, x may be between 0 and 5, between 0 and 2, between 0 and 3, between 0 and 4, between 1 and 5, between 1 and 3, between 1 and 4, or between 1 or 2, or may be 0, 1, 2, 3, 4, or 5. In some variations, y may be between 0 and 5, between 1 and 5, between 0 and 3, or between 1 and 3, or may be 0, 1, 2, 3, 4, or 5.

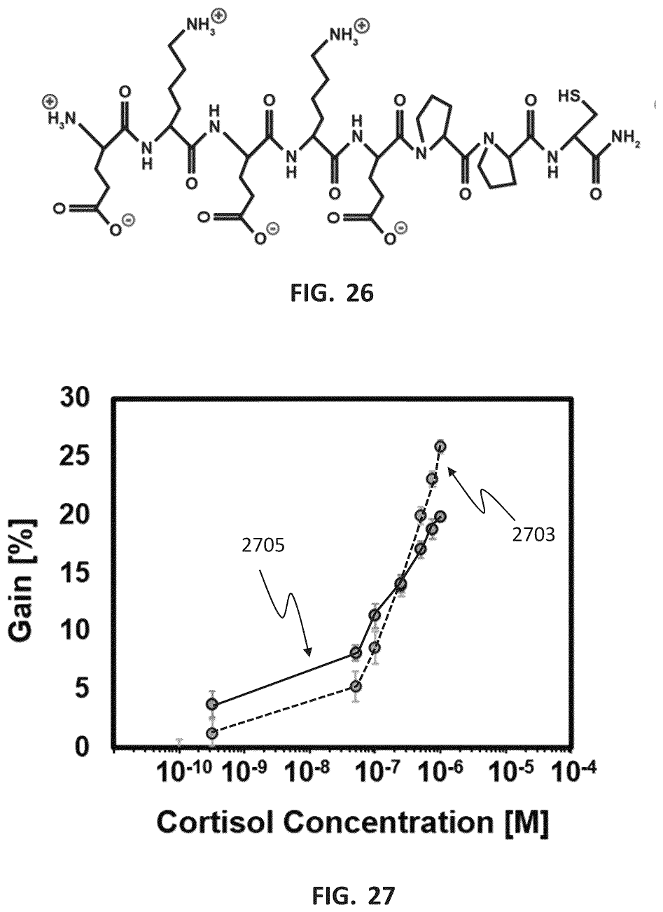

In some variations, the zwitterionic peptide may consist of the amino acid sequence of EKEKEPPC (SEQ ID NO: 2). In some variations, the zwitterionic peptide may consist of the amino acid sequence of EKEPPC (SEQ ID NO: 3), EKEKEPC (SEQ ID NO: 4), EKEPC (SEQ ID NO: 5), EPPC (SEQ ID NO: 6), EKEKEPPPC (SEQ ID NO: 7), EKEPPPC (SEQ ID NO: 8), or EPPPC (SEQ ID NO: 9). In some variations, the C-terminus of the C-terminal cysteine residue of SEQ ID NOS: 2-9 may be modified to be neutral in charge. By way of example, as provided in SEQ ID NOS: 10-17, the C-terminus of the C-terminal cysteine residue of SEQ ID NOS: 2-9 may be modified through amidation to be an amide group. An example of a zwitterionic peptide consisting of the amino sequence of SEQ ID NO: 2, with the C-terminal cysteine residue modified with an amide group at the C-terminus through amidation (SEQ ID NO: 10), is shown in FIG. 26. As shown there, the C-terminal cysteine provides a thiol group.

In some variations, the zwitterionic peptide may consist of the amino acid sequence of CPPKEKEK (SEQ ID NO: 18), CPPKEK (SEQ ID NO: 19), CPKEKEK (SEQ ID NO: 20), CPKEK (SEQ ID NO: 21), CPPK (SEQ ID NO: 22), CPPPKEKEK (SEQ ID NO: 23), CPPPKEK (SEQ ID NO: 24). CPPPK (SEQ ID NO: 25). In some variations, the N-terminus of the N-terminal cysteine residue of SEQ ID NOS: 18-25 may be modified to be neutral in charge. By way of example, as provided in SEQ ID NOS: 26-33, the N-terminus of the N-terminal cysteine residue of SEQ ID NOS: 18-25 may be modified through acetylation to be an amide group (specifically an acetamide group).

iii. Biocompatible Layer

In some variations, the working electrode may further comprise a biocompatible layer disposed on the biorecognition layer. The biocompatible layer may allow passage of small molecular weight analyte (e.g., cortisol) therethrough but may block passage of relatively high molecular weight biological components (e.g., albumin). In this manner, the biocompatible layer may enable the small molecular weight analyte to reach the analyte-binding aptamers while blocking unwanted high molecular weight components.

In some variations, the biocompatible layer may be a hydrogel comprising a hydrophilic polymer. The hydrophilic polymer may be selected from the group consisting of an agarose, a poly(urethane), a poly(N-vinylpyrrolidone), a poly(acrylamide), a poly(acrylic acid), a poly(methacrylic acid), a poly(2-hydroxyethyl methacrylate), a poly(acrylic acid-co-acrylamide), a poly(N-isopropyl acrylamide), a poly(2-acrylamido-2-methylpropane sulfonic acid), a poly(ethylene glycol), a poly(vinyl alcohol), a poly(lactic acid), a poly(glycolic acid), a poly(glycolic acid-co-lactic acid), a collagen, an alginate, a hyaluronic acid, a heparin, a glycosaminoglycans, a chitosan, Nafion, a carboxymethylcellulose, or a cellulose acetate. In some embodiments, the hydrophilic polymer may be an agarose. The agarose may have a sulfate content of less than about 0.5% w/w, less than about 0.4% w/w, less than about 0.3% w/w, less than about 0.2% w/w, and/or less than about 0.1% w/w. The agarose may be a low electroendosmosis agarose characterized by an electroendosmosis of between about 0.09 and about 0.14, between about 0.09 and between about 0.13, between about 0.09 and about 0.12, or between about 0.09 and about 0.11. In some variations, the agarose may have a melting point of between about 75 degrees Celsius (° C.) and about 97° C., between about 80° C. and about 95° C., or between about 85° C. and about 90° C. Agarose forms into a hydrogel based on a temperature transition where hydrogen bonds form and a physical structure emerges. This process is relatively mild compared to other polymers that require chemical cross-linking with reactive molecules to form bridges between the polymer strands. Chemical cross-linking and chemical polymerization typically require harsh chemicals, initiators, and possibly heat or UV light which could all contribute to damaging the sensor architecture and result in less robust and stable aptamer sensors. As such, using agarose advantageously reduces chemical wear and tear.

In some variations, the hydrogel may comprise, by way of example at the time the hydrogel is applied onto the biorecognition layer, the agarose at a concentration of between about 0.5% w/w and about 4% w/w or between about 0.5% w/w and about 2% w/w in a buffered saline solution, by way of example, in PBS. In some variations, the agarose may be dissolved in the buffered saline solution, by way of example a PBS, at a temperature of between about 85° C. and about 95° C., or about 90° C. In some variations, the hydrogel may have a pore size of between about 10 nm and about 1000 nm. Between about 100 nm and about 1000 nm. Or between about 10 nm and about 100 nm.

The hydrogel may further comprise an aqueous solvent into which the hydrophilic polymer is dissolved when the hydrogel is formed. The aqueous solvent may be water or a saline solution comprising one or more salts, by way of example NaCl, KCl, NazHPO₄, KH₂PO₄, or a combination thereof. In some embodiments, the saline solution may be a buffered saline, such as, for example. A phosphate buffered solution (PBS), an acetate buffer, a Tris buffer, a citric acid buffer, a McIlvaine buffer, a Tris-acetate-EDTA buffer, or a Tris-EDTA buffer.

The biocompatible layer may be applied over the biorecognition layer and/or diffusion limiting layer (if present) by means of at least one of spray coating, dip coating, chemical vapor deposition, drop casting, plasma vapor deposition, chemical vapor deposition, and electro-deposition.

A hydrogel is generally a continuous three-dimensional network of hydrophilic polymer with water dispersed throughout. The degree of hydration of the hydrogel may range from fully hydrated to fully desiccated. A fully hydrated hydrogel may be sufficiently hydrated so that the degree of hydration is in equilibrium with respect to an aqueous environment in which the hydrogel is placed. A partially hydrated (or partially desiccated hydrogel) will take up more water and thus “swell” when placed in an aqueous environment, whereas a fully hydrated hydrogel will not swell further. The partially desiccated hydrogel may be sufficiently desiccated so the degree of hydration of the hydrogel is in equilibrium with respect to the degree of humidity of the air the hydrogel (e.g., on a working electrode) is exposed to, by way of example, during storage. A fully desiccated hydrogel as used herein is substantially devoid of water. Generally, a desiccated hydrogel is capable of being rehydrated and be reconstituted into a fully hydrated state when placed into an aqueous environment. While the hydrogel of the biocompatible layer will generally be fully hydrated when the sensor is in use, the hydrogel of the biocompatible layer may be fully or partially desiccated during storage, i.e., after an electrode (or a sensor comprising one or more electrodes) is produced and prior to use on a subject.

In some variations, by way of example during storage, the hydrogel of the biocompatible layer may be at least partially desiccated. In some variations, the at least partially desiccated hydrogel may comprise about 10%, about 20%, about 30%, about 40%, about 50%, about 60%, about 70%, about 80%, or about 90% of water compared to a fully hydrated state of the hydrogel. In some variations, the at least partially desiccated hydrogel may be fully desiccated.

In some variations, the at least partially desiccated hydrogel may have a thickness has a thickness of between about 0.001 μm and about 1000 μm, between about 0.01 μm and about 100 μm, between about 0.1 μm and about 10 μm, between about 1 μm and about 50 μm, between about 5 μm and about 30 μm, between about 0.05 μm and about 500 μm, between about 0.1 μm and about 200 μm, or between about 1 μm and about 1000 μm (including all values and sub-ranges in any of the foregoing). It will be appreciated that the thickness of the hydrogel will depend on the degree of desiccation.

### Working Electrode—Exemplary Architectures

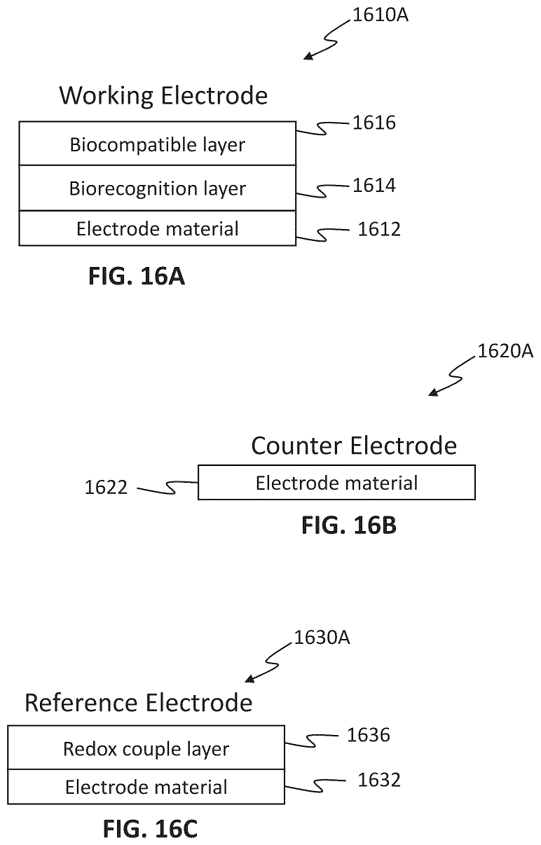

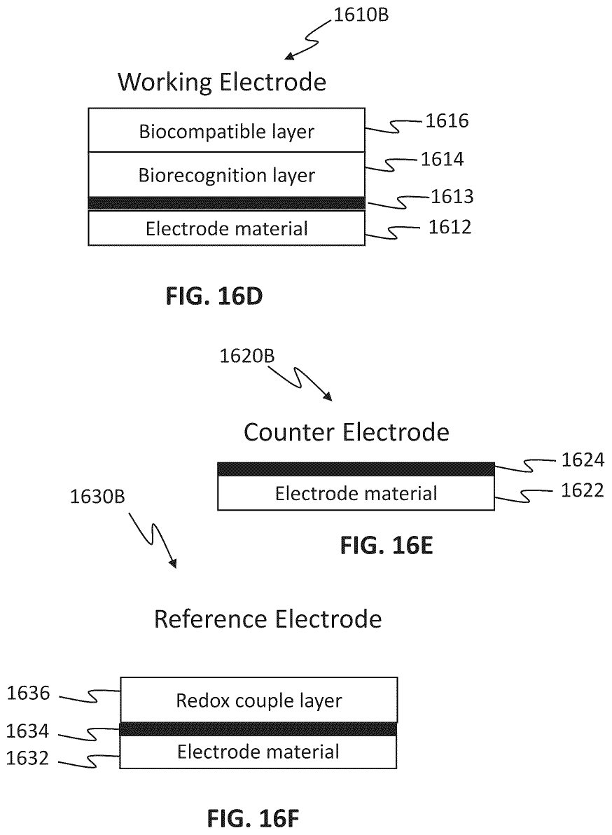

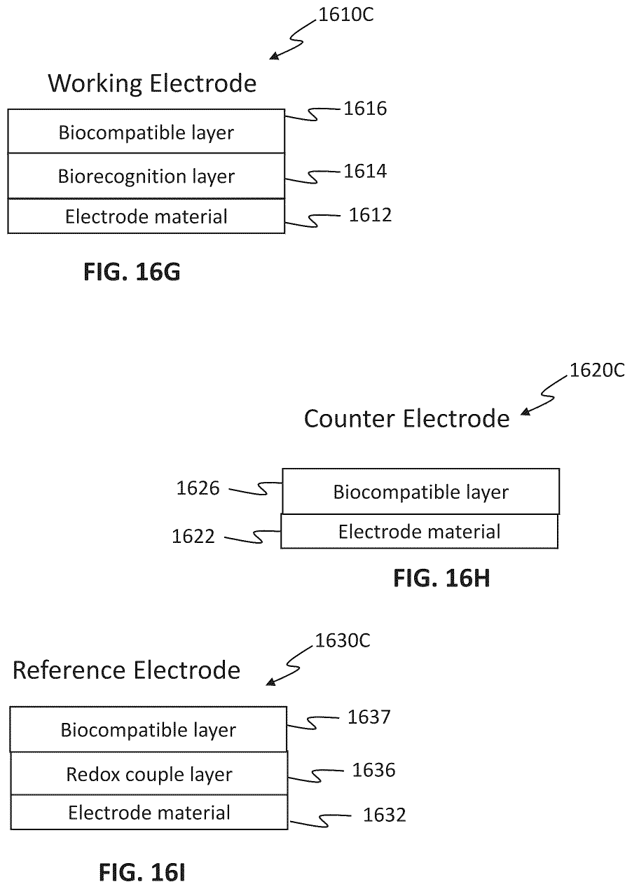

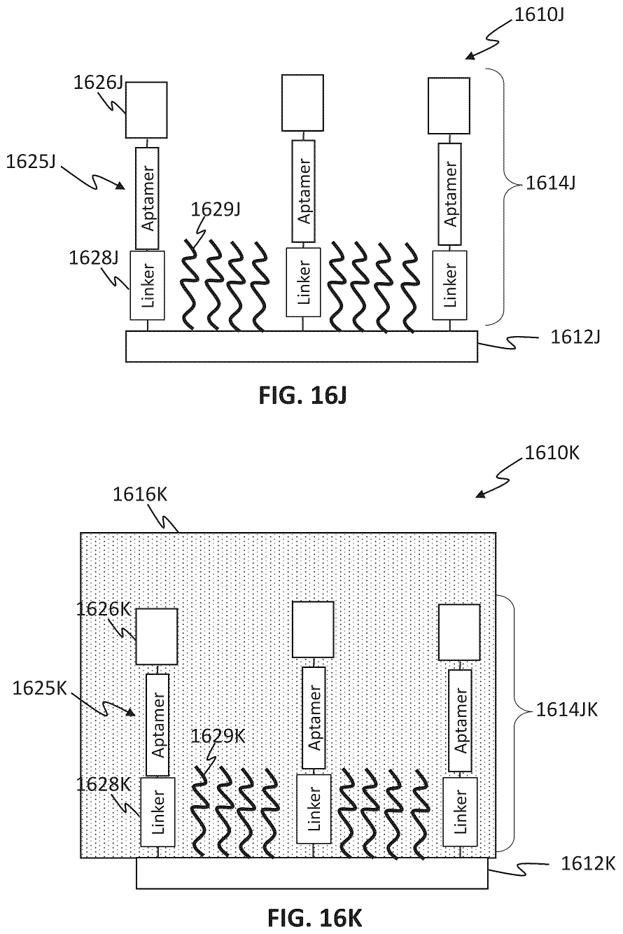

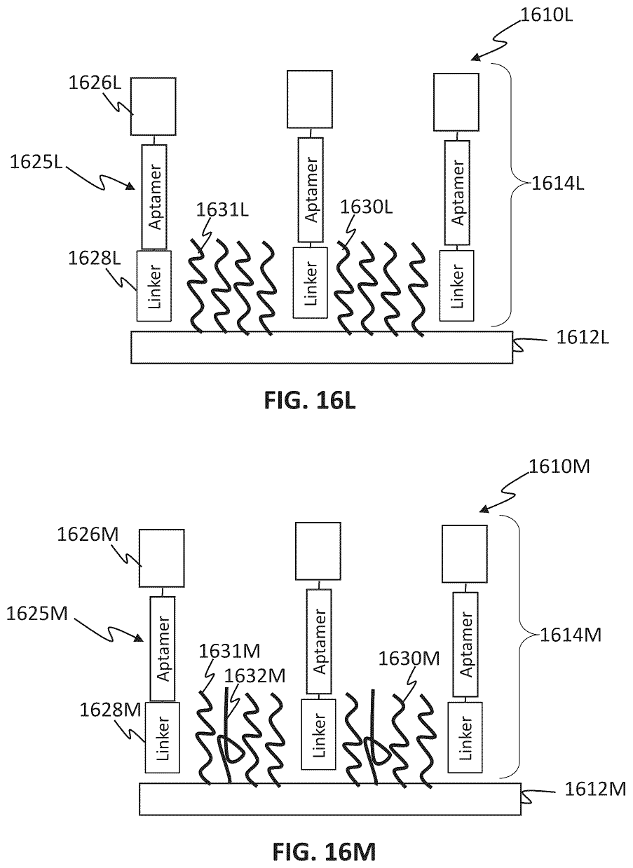

FIG. 16A depicts a schematic of an exemplary set of layers for an analyte-sensing working electrode 1610A. For example, as described above, in some variations the working electrode 1610A may include an electrode material 1612 and a biorecognition layer 1614 comprising a biorecognition element immobilized on the surface of the electrode material 1612. In some variations, the biorecognition layer 1614 further comprises a passivation element, which may include one or more of the small-molecule thiols described above, bound to the surface of the electrode material 1612 around the immobilized biorecognition element of the biorecognition layer 1614. In some variations, the biorecognition element may be an analyte-binding aptamer functionalized by a redox-active molecule. The electrode material 1612 may be used to electrically detect the change in electron transfer properties between the redox-active molecule attached to the analyte-binding aptamer comprised in biorecognition layer 1614 and the electrode material 1612 caused by analyte binding to the analyte-binding aptamer. The electrode material 1612 also provides ohmic contact and routes an electrical signal from the electrocatalytic reaction to processing circuitry. In some variations, the passivation element may comprise a multi-component passivation element (e.g., comprise two or more small-molecule thiols). In some variations, the electrode material 1612 may be platinum, as shown in FIG. 16A, or may be gold. In other variations, electrode material 1612 may include, for example, silver, palladium, iridium, rhodium, ruthenium, titanium, nickel, alloys of the aforementioned metals, cobalt chrome alloy, cobalt chrome molybdenum alloy, stainless steel 316L, iridium oxide, titanium nitride, carbon, doped diamond, boron-doped diamond, silicon, doped silicon, or other suitable material. The carbon may be pyrolytic carbon, pyrolytic graphite, or glassy carbon.

In some variations, the electrode material 1612 may be coated with a highly porous electrocatalytic layer, such as a platinum black layer 1613, which may augment the electrode surface area for enhanced sensitivity (as shown in FIG. 16D, for example). In some variations the platinum black layer 1613 may be omitted (as shown in FIGS. 16A and 16G, for example). In some variations, the electrode material 1612 may be, or include at the surface, unpolished electroplated gold, which has a rough surface and may thereby augment electrode surface area.

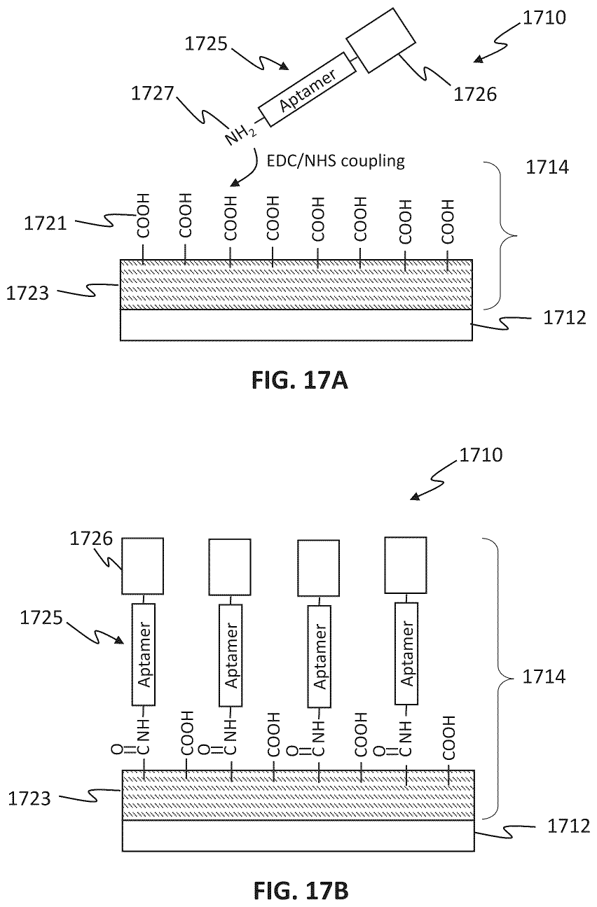

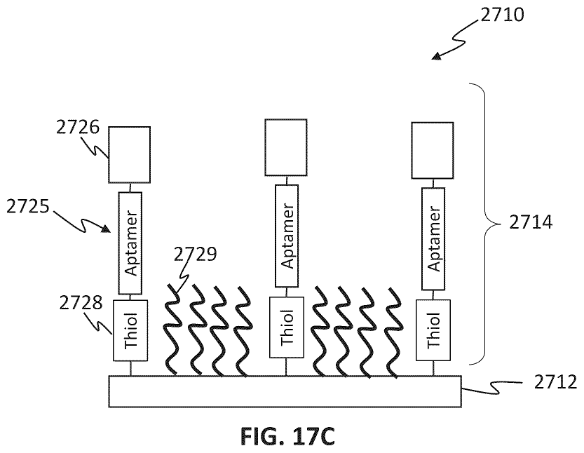

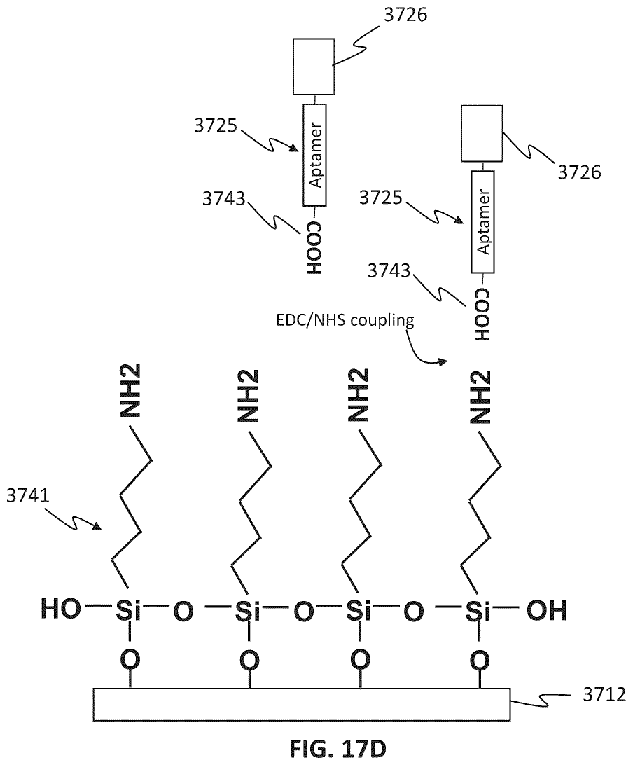

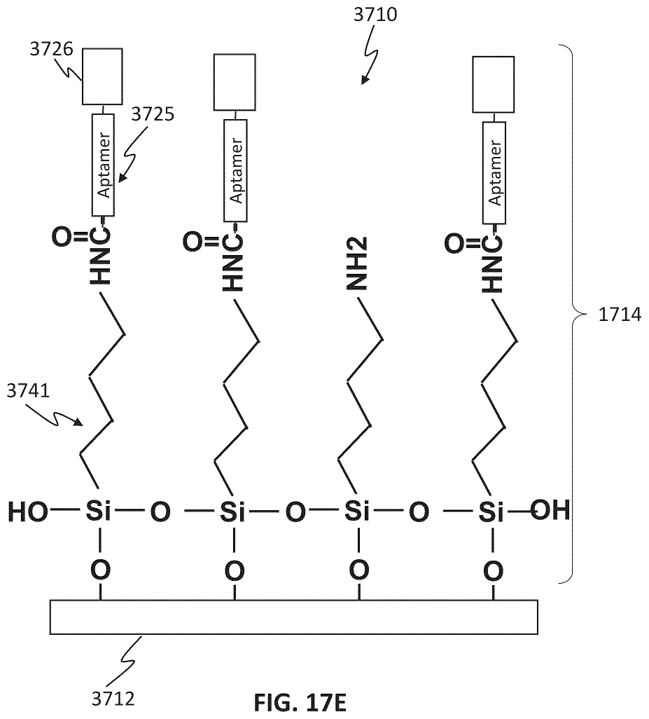

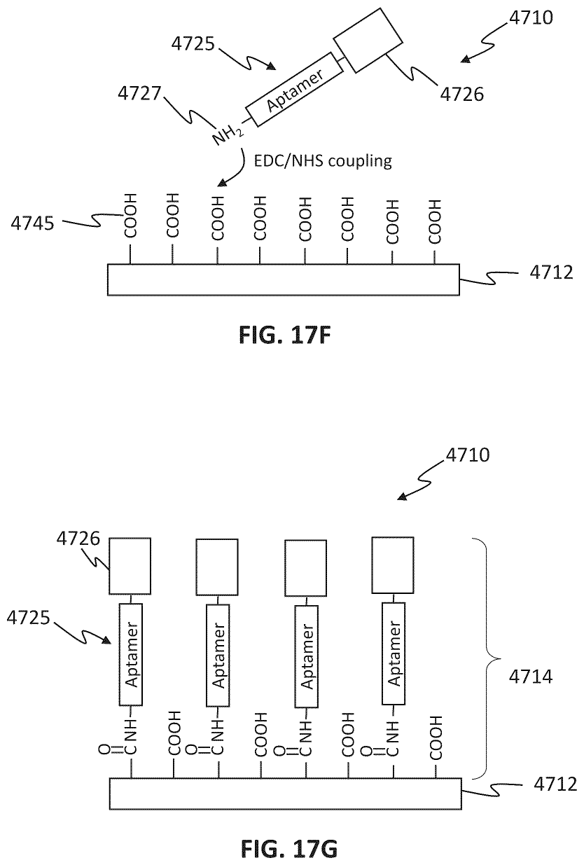

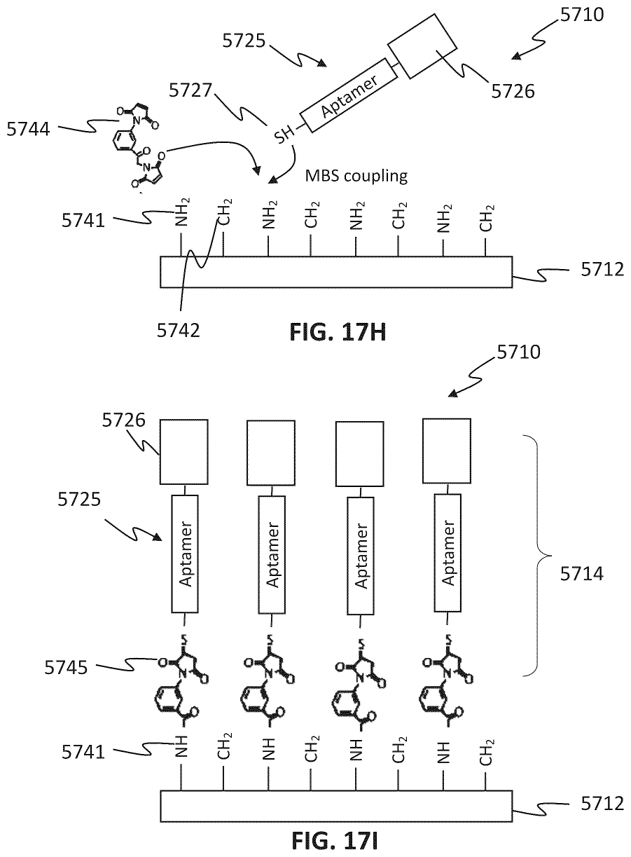

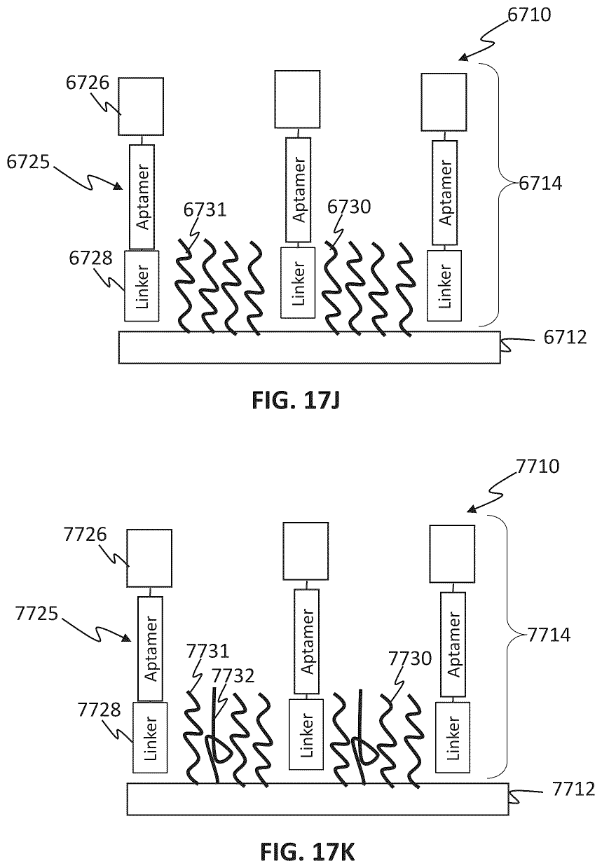

In some variations, the biorecognition layer 1614 may comprise a conducting polymer. The conducting polymer may be permselective to contribute to the biorecognition layer's robustness against circulating endogenous electroactive species (e.g., ascorbic acid, vitamin C, etc.), fluctuations of which may adversely affect the sensitivity of the sensor. Such a permselective conducting polymer in the biorecognition layer may further be more robust against pharmacological interferences (e.g., acetaminophen) in the interstitial fluid that may affect sensor accuracy. Conducting polymers may be made permselective by, for example, removing excess charge carriers by an oxidative electropolymerization process, or by intentionally overoxidizing the conductive polymer at an elevated potential after its polymerization, disrupting its conjugated backbone and rendering it non-conductive. These oxidatively-polymerized conducting polymers exhibit permselectivity and are hence able to reject ions of similar charge polarity to the dopant ion (net positive or negative) or by size exclusion due to the dense and compact form of the conducting polymers. In some variations, the conductive polymer may include one or more of aniline, pyrrole, pyrrole-3-carboxylic acid, pyrrole-1-propionic acid, acetylene, phenylene, phenylene vinylene, phenylenediamine, thiophene, 3-methylthiophene, 3-hexylthiophene, 3-thiophene carboxylic acid, 3,4-ethylenedioxythiophene (EDOT), EDOT carboxylic acid, and aminophenylboronic acid. An example of a working electrode in which a biorecognition layer 1714 comprises a film 1723 of conductive polymer is shown in FIGS. 17A and 17B.

In some variations, the conducting polymer may exhibit self-sealing and/or self-healing properties. For example, the conducting polymer may undergo oxidative electropolymerization, during which the conducting polymer may lose its conductivity as the thickness of the deposited conducting polymer on the electrode increases, until the lack of sufficient conductivity causes the deposition of additional conducting polymer to diminish. In the event that the conducting polymer has succumbed to minor physical damage (e.g., during use), the polymeric backbone may re-assemble to neutralize free charge and thereby lower overall surface energy of the molecular structure, which may manifest as self-sealing and/or self-healing properties.

Examples of a working electrode comprising electrode material and an aptamer-based analyte-sensing biorecognition layer are shown in FIGS. 16J-M, 17A-C, 17E, 17G and 17I.

Generally, an aptamer-based analyte sensor may comprise a working electrode comprising an electrode material, a passivation element, and analyte-sensing aptamers. The analyte-sensing aptamers and the passivation element may be disposed on the electrode material. The aptamers may be functionalized with a redox reporter molecule and tethered, directly or indirectly, to the electrode material. In some variations, the passivation element of FIG. 16J, FIG. 16L, and FIG. 16M may act to passivate an exposed surface of the electrode material from unwanted chemical reactions, such as, for example, the reduction of oxygen. The passivation element may also be used to provide a desired surface density of analyte-sensing aptamers on the surface of electrode material.

FIG. 16J shows an aptamer-based analyte sensor comprising a working electrode 1610J comprising a biorecognition layer 1614J including passivation element 1629J and analyte-sensing aptamers 1625J functionalized with a redox reporter molecule 1626J. The passivation element 1629J may comprise a single type of thiol-based small molecule and may be tethered thereby to an electrode material 1612J of the working electrode 1610J and the analyte-sensing aptamers 1625J may be tethered to the electrode material 1612J optionally via a linker 1628J. The redox reporter molecule 1626J may be, by way of example, methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine. The linker 1628J may be any functional group or molecule (including polymers) that provides a covalent link between an end of aptamer 1625J and the electrode material 1612J. The identity of the linker 1628J may be different based on the composition of the electrode material. For example, linker chemistries available for a platinum electrode surface may differ from linker chemistries for a gold electrode surface.

FIG. 16L shows an aptamer-based analyte sensor comprising a working electrode 1610L comprising a biorecognition layer 1614L including a multi-component passivation element and analyte-sensing aptamers 1625L functionalized with a redox reporter molecule 1626L. The multi-component passivation element may comprise a first small-molecule thiol 1630L and a second, different small-molecule thiol 1631L, each of which may be tethered to an electrode material 1612L of the working electrode 1610L. The analyte-sensing aptamers 1625L may be tethered to the electrode material 1612L optionally via a linker 1628. The redox reporter molecule 1626L may be, by way of example, methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine. The linker 1628L may be any functional group or molecule (including polymers) that provides a covalent link between an end of aptamer 1625L and the electrode material 1612L. The identity of the linker 1628L may be different based on the composition of the electrode material. For example, linker chemistries available for a platinum electrode surface may differ from linker chemistries for a gold electrode surface.

FIG. 16M shows an aptamer-based analyte sensor comprising a working electrode 1610M comprising a biorecognition layer 1614M including a multi-component passivation element and analyte-sensing aptamers 1625M functionalized with a redox reporter molecule 1626M. The multi-component passivation element may comprise a first small-molecule thiol 1630M, a second, different small-molecule thiol 1631M, each of which may be tethered to an electrode material 1612M of the working electrode 1610M, and a zwitterion 1632M (which may in some variations be a zwitterionic peptide or a thiol-based small molecule with a zwitterionic phosphorylcholine head group). The analyte-sensing aptamers 1625M may be tethered to the electrode material 1612M, for example, via a linker 1628M. The redox reporter molecule 1626M may be, by way of example, methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine. The linker 1628M may be any functional group or molecule (including polymers) that provides a covalent link between an end of the aptamer 1625M and the electrode material 1612M. The identity of the linker 1628M may be different based on the composition of the electrode material. For example, linker chemistries available for a platinum electrode surface may differ from linker chemistries for a gold electrode surface, as will be described in more detail herein.

Generally, linker chemistries, which may include thiols, silanes, covalent bonding chemistries, and oxidation-based amines, are selected according to the molecule to be linked and based on the particular electrode material used. For instance, thiols may attach on a number of electrode materials, including platinum electrodes. Silanes may attach to oxide films on the surface of a variety of electrode materials. Any carbon containing electrode material may form covalent bonds directly with organic molecules. For example, the carbon-based electrode material may be oxidized to carboxylic acids and then organic molecules can be attached with amines, forming amide bonds connecting them to the electrode material surface. Any of the electrode materials described herein may have a polymer deposited on their surface, which may be used as an insulating material and/or as a site for attachment of biorecognition elements. In these variations, the polymer may be a conducting polymer. Various examples of linker chemistries are described below with respect to FIGS. 17A-17K.

In a first example variation shown in FIG. 17B, the electrode material 1712 may be platinum, gold, silver, palladium, iridium, rhodium, ruthenium, titanium, nickel, alloys of the aforementioned metals, cobalt chrome alloy, cobalt chrome molybdenum alloy, stainless steel 316L, iridium oxide, indium oxide, titanium nitride, carbon, boron-doped diamond, doped diamond, silicon, doped silicon, or other suitable material. The carbon may be pyrolytic carbon, pyrolytic graphite, or glassy carbon.

The biorecognition layer 1714 may comprise a conducting polymer layer 1723 with analyte-binding aptamers 1725 tethered to the conducting polymer layer 1723, optionally via a linker. The linker may be an amide linker formed from a carboxyl group and a primary amine group. In some variations, the carboxyl group may be replaced with NHS-ester, isocyanate, isothiocyanate, or benzoyl fluoride. The present disclosure also provides for aptamers tethered via other linkers, such as a triazole linker, or a thioether linker. The thioether linker may be based on a combination of a maleimide moiety on the conductive polymer and a thiol moiety on an end of the aptamer, a combination of a vinyl surface attached to the conductive polymer layer and a thiol moiety on an end of the aptamer, or a combination of epoxide moiety on the conductive polymer and a thiol moiety on an end of the aptamer.

The analyte-binding aptamers 1725 may be functionalized with a redox-active molecule 1726, by way of example methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine.

The conducting polymer layer 1723 may be between about 1 nm and about 100 nm in thickness and comprises a conducting polymer and optionally a counter ion(s). Examples of the conducting polymer include one or more of aniline, pyrrole, pyrrole-3-carboxylic acid, pyrrole-1-propionic acid, acetylene, phenylene, phenylene vinylene, phenylenediamine, thiophene, 3-methylthiophene, 3-hexylthiophene, 3-thiophene carboxylic acid, EDOT, EDOT carboxylic acid, and aminophenylboronic acid. The conducting polymer may also be a copolymer of two or more of the monomers listed. Examples of the counter-ion(s) include chloride, phosphate, acetate, sulfate, bisulfate, nitrate, bromide, perchlorate, hexafluorophosphate, tetrafluoroborate, para-toluenesulfonate, benzenesulfonate, camphor-10-sulfonate, trifluoromethanesulfonate, bis(trifluoromethylsulfonyl)imide, dodecylbenzenesulfonate, poly(styrene sulfonate), poly(styrene sulfonate-co-acrylic acid), poly(acrylic acid), poly(methacrylic acid), poly(acrylic acid-co-acrylamide), sulfonated branched polytetrafluoroethylene (i.e. Nafion), poly(maleic acid), poly(maleic acid-co-acrylic acid), poly(maleic acid-co-acrylamide), poly(2-acrylamido-2-methylpropane sulfonic acid), poly(styrene sulfonate-block-butylene-ran-ethylene-block-styrene sulfonate), alginate, glycosaminoglycans, hyaluronic acid, collagen, and any combination of the aforementioned polymer as copolymers or block copolymers not explicitly mentioned.

In a second example variation shown in FIG. 17C, the electrode material 2712 may be gold, and the biorecognition layer 2714 may comprise analyte-sensing aptamers 2725 functionalized with a redox reporter molecule 2726, by way of example methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine. The analyte-sensing aptamers may be tethered to the electrode material 2712 via a thiol linker 2728. Optionally, the biorecognition layer 2714 may further comprise a passivation element comprising passivation molecules 2729. The passivation molecules 2729 may provide a desired surface density of analyte-sensing aptamers 2725 on the surface of electrode material 2712. In some variations, the passivation molecules 2729 may comprise small molecule thiols and/or zwitterions (e.g., zwitterionic peptides or thiol-based small molecules with a zwitterionic phosphorylcholine head group). as described herein, which may be tethered to the electrode material 2712.

In a third example variation shown in FIG. 17E, the electrode material 3712 may be a silicon that is optionally doped. Biorecognition layer 3714 may comprise a monolayer of silanes 3741 bound to the silicon surface, and analyte-sensing aptamers 3725 functionalized with a redox reporter molecule 3726 (by way of example methylene blue, ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine) that are tethered to the silicon electrode material optionally via amide linkers.

In a fourth example variation shown in FIG. 17G, the electrode material 4712 may be a carbon, optionally pyrolytic carbon, pyrolytic graphite, or glassy carbon. Biorecognition layer 4714 may comprise analyte-sensing aptamers 4725 functionalized with a redox reporter molecule 4726 (by way of example methylene blue (MB), ferrocene, pentamethyl ferrocene, C5-ferrocene, Nile blue, thionine, anthraquinone, C5-anthraquinone, gallocyanine, indophenol, neutral red, dabcyl, exTTF, or carboxy-X-rhodamine) that are tethered to the carbon electrode material optionally via amide linkers.

Whereas FIGS. 17B, 17C, 17E, and 17G (as well as FIGS. 17I-17K) show working electrodes with respective biorecognition layers without a biocompatible layer, it will be appreciated that each of the exemplary working electrodes may further comprise a biocompatible layer applied and disposed thereupon, as shown in FIG. 16K. The biocompatible layer may be a hydrogel biocompatible layer, as described below.

As shown in FIGS. 16A, 16D, 16G, and 16K, in some variations, the working electrode may further include a biocompatible layer 1616. The biocompatible layer 1616 may allow passage of small molecular weight analyte (e.g., cortisol) to reach the analyte-binding aptamers 1625 while blocking passage of relatively high molecular weight biological components, such as, for example, cells, cell debris, or macromolecular aggregations. The size selectivity of biocompatibility layer 1616 depends on aspects of the material comprising the layer, by way of example pore size. Such selective passage may prevent or mitigate non-specific adsorption and accumulation of high molecular weight biological components to the biorecognition layer 1614. Adsorption and/or accumulation of relatively high molecular weight biological components may interfere with the structure-switching mechanism of the analyte-sensing aptamers 1625 and degrade responsiveness of the working electrode. The biocompatible layer 1616 may also, for example, reduce the foreign body response.

The biocompatible layer 1616 may generally be applied on the biorecognition layer 1614 after the biorecognition layer 1614 has been deposited (i.e., after the biorecognition layer is already in place). As such, the biocompatible layer 1616 is schematically depicted in FIGS. 16A, 16D, and 16G as disposed on and above the biorecognition layer 1614. That being said, the biorecognition layer, as shown for example in FIGS. 17A-17K, may be a nanoscale layer consisting of certain molecular or macromolecular components, and may be substantially thinner than the biocompatible layer. As such, it will be appreciated that the depiction of the biorecognition layer 1614 in FIGS. 16A, 16D, and 16G are schematic in nature and may not reflect actual size.

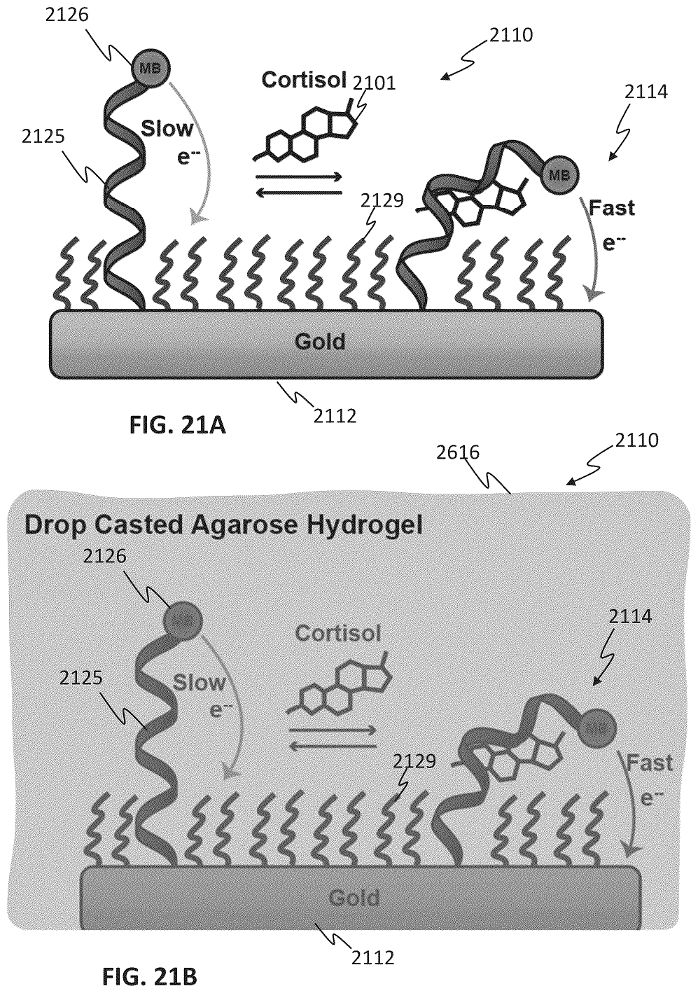

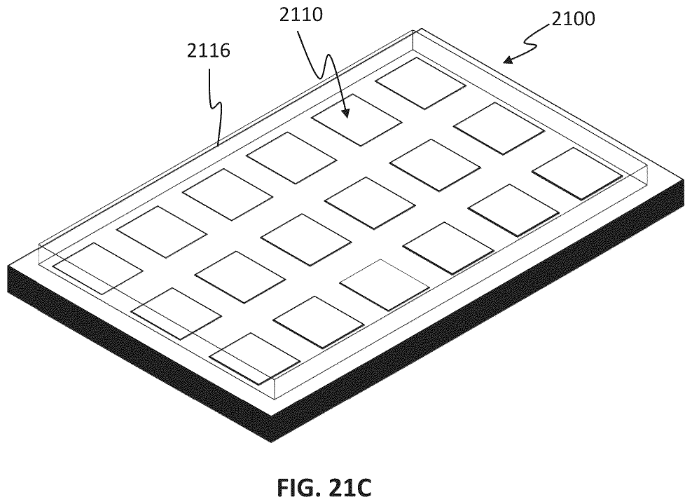

In some variations the biocompatible layer may be individually applied on each working electrode. In some variations, the biocompatible layer may be disposed generally on all or a portion of an electrode and/or microneedle array comprising a plurality of electrodes. As a result, the biocompatible layer may have a substantially larger surface area than an individual electrode. It will be appreciated that the biocompatible layer may in fact extend laterally beyond the surface of a given electrode. Moreover, in some variations, multiple electrodes may be covered with a same biocompatible layer, by way of example as shown in FIG. 21C.

In some variations, the application of the biocompatible layer may be limited to portions of the electrode array comprising working electrodes. FIGS. 16A-16C by way of example show a working electrode 1610A (FIG. 16A) comprising a biocompatible layer 1616 disposed on the biorecognition layer 1614, and a counter electrode 1620A (FIG. 16B) and a reference electrode 1630A (FIG. 16C) each lacking a biocompatible layer. In other variations, the biocompatible layer may be applied generally to one or more surfaces (e.g., skin-facing or penetrating surfaces) of an electrode array or microneedle array, so that one or more of the counter and reference electrodes, including, for example, all electrodes in the array, comprise a biocompatible layer. FIGS. 16G-16I by way of example show each of a working electrode 1610C (FIG. 16G), a counter electrode 1620C (FIG. 16H), and a reference electrode 1630C (FIG. 16I) each comprising a biocompatible layer.

### Counter Electrode

As described above, the counter electrode is the electrode that is sourcing or sinking electrons (via an electrical current) required to sustain the electrochemical reaction at the working electrode. The number of counter electrode constituents can be augmented in the form of a counter electrode array to enhance surface area such that the current-carrying capacity of the counter electrode does not limit the change in electron transfer properties between the redox-active molecule and the electrode material 1612 of the working electrode. It thus may be desirable to have an excess of counter electrode area versus the working electrode area to circumvent the current-carrying capacity limitation. If the working electrode is operated as an anode, the counter electrode will serve as the cathode and vice versa. Similarly, if an oxidation reaction occurs at the working electrode, a reduction reaction occurs at the counter electrode and vice versa. Unlike the working or reference electrodes, the counter electrode is permitted to dynamically swing to electrical potentials required to sustain the change in electron transport properties of the working electrode.

As shown in FIG. 16B, a counter electrode 1620A may include an electrode material 1622, similar to electrode material 1612. For example, like the electrode material 1612, the electrode material 1622 in counter electrode 1620A may include gold, platinum, palladium, iridium, rhodium, ruthenium, titanium, nickel, alloys of the aforementioned metals, cobalt chrome alloy, cobalt chrome molybdenum alloy, stainless steel 316L, iridium oxide, titanium nitride, carbon, doped diamond, silicon, doped silicon, or other suitable material.

In some variations, the counter electrode may have few or no additional layers over the electrode material 1622 (for example counter electrode 1620A as shown in FIG. 16B). However, in some variations the counter electrode 1620 may benefit from increase surface area to increase the amount of current it can support. For example, the counter electrode material 1622 may be textured or otherwise roughened in such a way to augment the surface area of the electrode material 1622 for enhanced current sourcing or sinking ability. Additionally, or alternatively, the counter electrode may include a layer of platinum black 1624 as shown in FIG. 16E as counter electrode 1620B, which may augment electrode surface as described above with respect to some variations of the working electrode. However, in some variations of the counter electrode, the layer of platinum black may be omitted (by way of example counter electrode 1620A and 1620C as shown in FIGS. 16B and 16H, respectively).

Additionally, or alternatively, in some variations as shown in FIG. 16H, the counter electrode 1620C may include a biocompatible layer 1626 arranged over the electrode in order to, for example, reduce the foreign body response. The biocompatible layer 1626 may, for example, be similar in structure and composition to the biocompatible layer 1616 described above with respect to FIG. 16A.

### Reference Electrode

As described above, the reference electrode functions to provide a reference potential for the system; that is, the electrical potential at which the working electrode is biased is referenced to the reference electrode. A fixed or at least controlled potential relationship may be established between the working and reference electrodes, and within practical limits no current is sourced from or sinked to the reference electrode.

As shown in FIG. 16C, a reference electrode 1630A may include an electrode material 1632, similar to electrode material 1612. In some variations, like the electrode material 1612, the electrode material 1632 in the reference electrode 1630A may include a metal salt or metal oxide, which serves as a stable redox coupled with a well-known electrode potential. For example, the metal salt may, for example, include silver-silver chloride (Ag/AgCl) and the metal oxide may include iridium oxide (IrOx/Ir₂O₃/IrO₂). In other variations, noble and inert metal surfaces, and those coated with a conductive polymer such as poly(3,4-ethylenedioxythiophene), may function as quasi-reference electrodes and include gold, platinum, palladium, iridium, carbon, doped diamond, and/or other suitable catalytic and inert material. Furthermore, in some variations the reference electrode 1630A may be textured or otherwise roughened in such a way to enhance adhesion with any subsequent layers. Such subsequent layers on the electrode material 1632 may include a platinum black layer 1634 (as shown in FIG. 16F as reference electrode 1630B), and/or biocompatible layer 1637 (as shown in FIG. 16I as reference electrode 1630C). The biocompatible layer 1637 may, for example, be similar to the biocompatible layer 1616 described above with respect to FIG. 16A. In some variations, the platinum black layer may be omitted (e.g., as shown in FIGS. 16C and 16I as reference electrodes 1630A and 1630B, respectively).

The reference electrode 1630A may, in some variations, further include a redox-couple layer 1636, which may contain a surface-immobilized, solid-state redox couple with a stable thermodynamic potential. For example, the reference electrode may operate at a stable standard thermodynamic potential with respect to a standard hydrogen electrode (SHE). The high stability of the electrode potential may be attained by employing a redox system with constant (e.g., buffered or saturated) concentrations of each participant of the redox reaction. For example, the reference electrode may include a metal with an Ag/AgCl salt film (E=+0.197V vs. SHE) or IrOx (E=+0.177 vs. SHE, pH=7.00) in the redox-couple layer 1636. In some variations, the reference electrode may be used as a half-cell to construct a complete electrochemical cell.

### Electrode(s)—Methods

Various layers of the working electrode, counter electrode, and reference electrode may be applied to the microneedle array and/or functionalized, etc. using suitable processes such as those described below.

In a pre-processing step for the microneedle array, the microneedle array may be plasma cleaned in an inert gas (e.g., RF-generated inert gas such as argon) plasma environment to render the surface of the material, including the electrode material (e.g., electrode material 1612, 1622, and 1632 as described above), to be more hydrophilic and chemically reactive. This pre-processing functions to not only physically remove organic debris and contaminants, but also to clean and prepare the electrode surface to enhance adhesion of subsequently deposited films on its surface.

### Working Electrode

Anodization: To configure the working electrode after the pre-processing step, the electrode material 1612, in some variations, may undergo an anodization treatment using an amperometry approach. For example, the electrode constituent(s) assigned for the working electrode function may be subject to a fixed high anodic potential (e.g., between +1.0 V and +1.3 V vs. Ag/AgCl reference electrode) for a suitable amount of time (e.g., between about 30 sec and about 10 min) in a moderate-strength acid solution (e.g., between 0.1 M and 3M H₂SO₄). In this process, a thin, yet stable native oxide layer may be generated on the electrode surface. Owing to the low pH arising at the electrode surface, any trace contaminants may be removed as well.

In an alternative embodiment using a coulometry approach, anodization can proceed until a specified amount of charge has passed (measured in Coulombs). The anodic potential may be applied as described above; however, the duration of this might vary until the specified amount of charge has passed.

Activation: Following the anodization process, the working electrode constituents, in some variations, may be subjected to a cyclically-scanned potential waveform in an activation process using cyclic voltammetry (CV). In the activation process, which may occur in a moderate-strength acid solution (e.g. 0.1-3M H₂SO₄), the potential applied may time-varying in a suitable function (e.g., sawtooth or triangular function). For example, the voltage may be linearly scanned between a cathodic value (e.g., between −0.3 V and −0.2 V vs. Ag/AgCl reference electrode) and an anodic value (e.g., between +1.0 V and +1.3 V vs. Ag/AgCl reference electrode) in an alternating function (e.g., between 15 and 50 linear sweep segments). In one example, the working electrode constituents may be subjected to high-speed CV (at approximately 500 mV/s) followed by two rounds of slow CV (at approximately 100 mV/s), and changing the acid solution between each step. The scan rate of this waveform can take on a value between 1 mV/sec and 1000 mV/sec. It should be noted that a current peak arising during the anodic sweep (sweep to positive extreme) corresponds to the oxidation of a chemical species, while the current peak arising during the ensuing cathodic sweep (sweep to negative extreme) corresponds to the reduction of said chemical species.

Electrodeposition of gold: Following the activation and cleaning process, the working electrode constituents, in some variations, may be subjected to a cyclically-scanned potential waveform or a constant potential to electrodeposit gold metal onto the surface. In the deposition process, which may occur in dilute concentrations of gold complexes (e.g., 0.5% to 3% by weight AuCl₃ or Au(CN)₂), the potential applied may vary with time in a suitable function. For example, the voltage may be linearly scanned between an anodic value (e.g., between +0.2 V and +1.2 V vs. Ag/AgCl reference electrode) in an alternating function (e.g., 10 to 30 linear sweep segments). The scan rate of this waveform can take on a value between 100 mV/sec and 1000 mV/sec. It should be noted that a rise in current during the cathodic sweep (sweep to negative extreme) corresponds to the reduction of gold species in solution and plating to the working electrode surface. It should also be noted that a sharp increase in current during the first scan is due to the formation of nucleation sites. In some variations, the gold may be deposited through physical vapor deposition.

Activation of the gold surface: Depending on the deposition method of gold on the working electrode (provided that gold is used), the electrode, in some variations, may require electrochemical activation. Activation may begin in a basic solution (e.g., 0.1 M to 3M NaOH), and the potential applied may be time-varying in a suitable function (e.g., triangle function) such as cyclic voltammetry. For example, the voltage may be linearly scanned between a cathodic value (e.g., between −2.0 V and −1.2 V vs. Ag/AgCl reference electrode) and an anodic value (e.g., between −1.1 V and −0.5 V vs. Ag/AgCl) in an alternating function (e.g., 50 to 1000 sweep segments). The scan rate of this waveform can take on a value between 10 mV/s and 2000 mV/s. After potential cycling in alkaline media the gold electrodes must be cycled in an acidic solution (e.g., between 0.1 M and 3 M H₂SO₄). The potential may be time-varying in a suitable function (e.g., triangle function). For example, the voltage me be linearly scanned between a cathodic value (e.g., between −0.2 V and +0.2 V vs. Ag/AgCl reference electrode) and an anodic value (e.g., between +1.2 V and +1.8 V vs. Ag/AgCl reference electrode) in an alternating function (e.g., 50 to 1000 sweep segments). The scan rate of this waveform can take on a value between 10 mV/s and 2000 mV/s. It should be noted that in both basic and acidic media, a current peak arising during the anodic sweep (sweep to positive extreme) corresponds to the oxidation of a chemical species, while the current peak arising during the ensuing cathodic sweep (sweep to negative extreme) corresponds to the reduction of said chemical species.

Functionalization of the biorecognition layer: Following the activation process, the working electrode constituents may, in some variations, be functionalized with the biorecognition layer 1614 such as that described above. Assuming that the working electrode contingent of the microneedle array has undergone the aforementioned steps such as deposition and activation, the biorecognition layer may be applied in a variety of ways. The application process for the biorecognition layer may depend on various factors, including what material is used for the working electrode material 1612. Various exemplary processes are discussed herein below.

In some variations, the electrode material 1612 is electrochemically coated with a conductive polymer film through oxidation of the reactive monomer(s) at the electrode surface, either potentiostatically (e.g., chronoamperometry), potentiodynamically (e.g., cyclic voltammetry), or galvanostatically (e.g. chronopotentiometry). The conditions necessary for polymerization of the monomer(s) is dependent on chemical properties of the conductive polymer being used. By adjusting current, potential, sweep rate, and/or the duration of the electrolytic process, the final properties of the conductive polymer film may be controlled (e.g., thickness, conductivity, permselectivity). When desired, the conductive polymer film may be overoxidized with a secondary oxidation step through a potentiostatic, potentiodynamic, or galvanostatic electrochemical process. This secondary oxidation step may be used to alter electrical conductivity and barrier properties of the conductive polymer film. The conductive polymer and/or counter ion(s) incorporated within the conductive polymer may provide pendent chemical moieties for covalent attachment to aptamers functionalized with a compatible moiety. By way of example, the pendant chemical moiety in the conductive polymer film may comprise a carboxyl group and the aptamer may be functionalized on 3′ or 5′ end with a primary amine. Alternatively, the pendant chemical moiety in the conductive polymer film may be a primary amine and the aptamer may be functionalized on 3′ or 5′ end with a carboxyl group.

In some variations, by way of example where the electrode material 1612 is gold, the aptamer may be functionalized on one terminal end with a reactive organosulfur compound (by way of example thiol/mercaptan, disulfide), which spontaneously reacts and attaches to the surface of the gold working electrode material. The remaining gold surface of the working electrode may optionally be passivated with e.g., small molecule(s) containing a terminal thiol group to form a self-assembled monolayer.

In an example variation as shown in FIG. 17A, the working electrode 1710 may be functionalized with an aqueous solution comprising a monomeric precursor of a conducting polymer and optionally a counter ion. Functionalization may take place through a galvanostatic electrodeposition (e.g., chronopotentiometry) where the anodic current density through the working electrode may be between 0.05 mA/cm² and 5 mA/cm² and the duration may be from about 1 minute to about 100 minutes. In other variations, the functionalization may take place through a chronocoulometric deposition, where the deposit charge may be between about 1 mC/cm² and 10 C/cm² and the duration may be from about 1 minute to about 100 minutes. The conducting polymer, the counter ion, or both may comprise carboxyl group 1721. In this process, as shown in FIG. 17A, a thin film (e.g., between about 10 nm and about 100 nm) of biorecognition layer 1714 comprising a conducting polymer layer 1723 may be generated (e.g., electrodeposited or electropolymerized) on the surface of electrode material 1712, which may include platinum. After the conducting polymer layer 1723 is formed, analyte-sensing aptamers 1725 functionalized with a primary amine group 1727 on one end and functionalized with a redox-active molecule 1726 on the other end are introduced. The aptamers 1725 are covalently linked to the carboxyl groups 1721 comprised in the conductive polymer layer 1723 using a carbodiimide cross-linking method, by way of example EDC/NHS coupling that employs 1-ethyl-3-(3-dimethylaminopropyl) carbodiimide hydrochloride (EDC) and N-Hydroxysuccinimide (NHS). An alternative method for coupling a primary amine and carboxylic acid into an amide linkage is through DMTMM cross-linking with 4-(4,6-dimethoxy-1,3,5-triazin-2-yl)-4-methyl-morpholinium chloride. The remainder of pendent activated carboxylic acid groups on the silane layer not attached to aptamer may optionally be cross-linked to another amine containing molecule (e.g., ethanolamine, glycine, lysine, PEG-amine) to alter the working electrode surface properties and biocompatibility. The resulting functionalized working electrode 1710 is shown in FIG. 17B.

In some variations, the placement of the carboxyl group and the primary amine group for the carbodiimide cross-linking may be reversed, such that conducting polymer, the counter ion, or both may comprise the primary amine group and the aptamer may be functionalized with the carboxyl group. The two moieties may then be linked by EDC/NHS coupling or DMTMM cross-linking as noted above with respect to FIG. 17A. An example of a conducting polymer comprising a primary amine group is a p-Phenylenediamine (PPD)-based polymer.

Whereas FIGS. 17A and 17B show aptamers tethered via an amide linker formed between a carboxyl group and a primary amine group, the present disclosure provides for the use of other moieties. By way of example, the carboxyl group may be replaced with NHS-ester, isocyanate, isothiocyanate, or benzoyl fluoride. The present disclosure also provides for aptamers tethered via other linkers, such as a triazole linker, or a thioether linker. The thioether linker may be based on a combination of a maleimide moiety on the conductive polymer and a thiol moiety on an end of the aptamer, a combination of a vinyl surface attached to the conductive polymer layer and a thiol moiety on an end of the aptamer, or a combination of epoxide moiety on the conductive polymer and a thiol moiety on an end of the aptamer.

In an example variation shown in FIG. 17D, a working electrode 3710 comprising a silicon electrode material 3712 may be functionalized with an aptamer 3725 via silane chemistry. The silicon electrode material may comprise doped silicon. First, the surface of the silicon electrode material 3712 is activated by exposure to oxygen plasma at a pressure between about 200 mTorr and about 700 mTorr for about 0.5 min to about 10 min, at an operating power of about 50 W to about 500 W. The activated electrode material surface is then moved to a vacuum chamber at a pressure of about 1 Torr to about 100 Torr containing a small volume of a suitable reactive silane 3741 comprising a terminal primary amine (by way of example 4-aminobutyltriemethoxysilane as shown in FIG. 17D) for 10 to 90 min. Afterwards, the working electrodes are removed and rinsed with an organic solvent(s) (by way of example methanol, ethanol, or isopropanol) then baked at about 80° C. to about 140° C. for a duration of about 10 min to about 60 min to cure the adhered silane 3741. FIG. 17D schematically shows a silicon electrode material 3712 with a monolayer of adhered silanes 3741. Also as shown in FIG. 17D, the electrode material is treated with a solution containing an aptamer 3725 terminating with a carboxyl group 3743 at one end and a redox active molecule 3726 on the other end. The solution may be an aqueous solution such as a phosphate buffered solution, with the aptamers at a concentration of between about 0.1 μM and about 2 μM. The carboxyl group 3743 forms an amide linker with the pendent amine groups of the adhered silanes 3741, resulting in the aptamer 3725 being tethered to the electrode material 3712. The resulting biorecognition layer 3714 comprising the tethered aptamers 3725 and silanes 3741 that is formed on the silicon electrode material 3712 is shown in FIG. 17E. The amide linker may be formed via an EDC/NHS cross-linking chemistry, or a DMTMM cross-linking chemistry. The remainder of pendent amine groups (not shown) on the silane layer not attached to an aptamer may optionally be cross-linked to another carboxyl group-containing molecule (e.g. glycolic acid, oxalic acid, glycine, PEG-carboxylic acid) to alter the working electrode surface properties and biocompatibility.

In an example variation shown in FIG. 17F, a working electrode 4710 comprising a glassy carbon electrode material 4712 may be functionalized with an aptamer via activation of the carbon surface and formation of an amide bond that tethers the aptamer to the carbon surface. In a first step, the surface of the glassy carbon electrode material 4712 is activated electrochemically by a potentiostatic hold at 1.2 V to 2.0 V for 1 to 60 min in an acidic solution (by way of example a 0.1 M to 3 M H₂SO₄ solution). The activation step forms terminal carboxyl groups 4745 on the surface of the carbon electrode material 4712. The activated surface of the electrode material 4712 is then functionalized with an aptamer 4725 having a primary amine group 4727 at its 3′ or 5′ end and a redox active molecule 4726 on the other end. The carboxyl groups 4745 form an amide linker with the amine groups 4727, resulting in the aptamers 4725 being tethered to the electrode material 4712. The resulting biorecognition layer 4714 comprising the tethered aptamers 4725 that is formed on the carbon electrode material 4712 is shown in FIG. 17G. The amide linker may be formed via an EDC/NHS cross-linking chemistry, or a DMTMM cross-linking chemistry.

Whereas FIGS. 17D-17G show aptamers tethered via an amide linker formed between a carboxyl group and a primary amine group, the present disclosure provides for the use of other moieties. By way of example, the carboxyl group may be replaced with NHS-ester, isocyanate, isothiocyanate, or benzoyl fluoride. The present disclosure also provides for aptamers tethered via other linkers, such as a triazole linker, or a thioether linker. The thioether linker may be based on a combination of a maleimide moiety on the conductive polymer and a thiol moiety on an end of the aptamer, a combination of a vinyl surface attached to the conductive polymer layer and a thiol moiety on an end of the aptamer, or a combination of epoxide moiety on the conductive polymer and a thiol moiety on an end of the aptamer.

With reference to FIG. 17C, FIG. 17J, and FIG. 17K, functionalization of the biorecognition layer 2714, 6714, 7714 may include deposition of each of an aptamer 2725, 6725, 7725 and a passivation element comprising at least one thiol-based small molecule 2729, 6730, 6731, 7730, 7731 and/or a zwitterion 7732 (which may in some variations be a zwitterionic peptide or a thiol-based small molecule with a zwitterionic phosphorylcholine head group). In this way, a portion of a surface of electrode material 2712, 6712, 7712 comprises aptamer and another portion comprises the passivation element, which may include thiol-based small molecules and/or zwitterions (e.g., zwitterionic peptides or thiol-based small molecules with a zwitterionic phosphorylcholine head group) in order to passivate the surface and/or add biocompatible functionality. For instance, as shown in FIG. 17C, a small molecule thiol 2729 fills a surface of an electrode material 2712 not occupied by an aptamer 2725.

To this end, FIG. 18A and FIG. 18B provide a flow diagram of methods of depositing a passivation element comprising at least one thiol-based small molecule to a surface of an electrode material. Under either method 1810A or method 1810B, after completion of pre-processing, anodization, and activation of the electrode material, which may each be performed as described above, the working electrode constituents may be functionalized with the biorecognition layer. Though described below in one exemplary order, it should be appreciated that a biorecognition element (e.g., an aptamer) and a passivation element may be deposited concurrently or sequentially. For instance, the biorecognition element may deposited before the passivation element, as described below, or the passivation element may be deposited before the biorecognition element, as desired.

It should also be appreciated that the functionalization described herein may include polymerization of a polymer that entraps a biorecognition element therein. In some cases, when the biorecognition layer comprises a conducting polymer, a voltage may be linearly scanned between a cathodic value (e.g., between −0.5 V to 0.0 V vs. Ag/AgCl reference electrode) and an anodic value (e.g., between 0.5 V+1.5 V vs Ag/AgCl reference electrode) in an alternating function (e.g., 10 linear sweep segments). In an example variation, the scan rate of this waveform can take on a value between about 1 mV/sec and about 1,000 mV/sec in an aqueous solution comprised of a monomeric precursor to the entrapment conducting polymer and a biorecognition element (e.g., aptamer). The biorecognition element may be present in the aqueous solution at a concentration of between 0.1 μM and about 2 μM. In this process, a thin film (e.g., between about 10 nm and about 1000 nm) of a biorecognition element-laden polymer may be generated (e.g., electrodeposited or electropolymerized) on the working electrode surface.

Methods 1810A and 1810B, however, will be described with reference to a biorecognition layer lacking a polymerized polymer network. To this end, in variations where the biorecognition layer does not comprise a polymer, depositing the biorecognition element (e.g., aptamer) at step 1820A of method 1810A and step 1820B of method 1810B comprises submerging the electrode material in a solution comprising aptamer. The electrode material may be submerged for a pre-determined period of time of between about 3 hours and about 30 hours and the concentration of the aptamer in the solution may be between about 0.1 μM and about 2 μM, between about 0.2 μM and about 1 μM, between about 0.3 μM and about 0.75 μM, or between about 0.4 μM and about 0.6 μM.

With reference now to FIG. 18A, and following deposition of the biorecognition element at step 1810A, a first thiol-based small molecule may be deposited (i.e., applied) at step 1830A of method 1810A. The first thiol-based small molecule may be any one of the thiol-based small molecules described herein and may be deposited according to any suitable method, including but not limited to drop casting, printing, spray coating, soaking, spin coating, and chemical deposition. For instance, the first thiol-based small molecule may be deposited by soaking, or submerging, the electrode in a first solution. In some variations, the first solution may comprise the first thiol-based small molecule in a first solvent. The first solvent may comprise an organic solvent or an inorganic solvent. For instance, the first solvent may be one or more solvents selected from the group consisting of ethanol, methanol, propanol, isopropanol, butanol, acetone, acetonitrile, dimethylsulfoxide, dimethylformamide, dimethylacetamide, propylene carbonate, ethylene carbonate, deionized water, phosphate buffered saline (PBS), and a buffered salt solution. In an example, the first solvent may be ethanol. In an example, the first solvent may be a mixture of methanol or ethanol and deionized water, PBS, or a buffered salt solution. For instance, the mixture comprise methanol and deionized water, methanol and PBS, methanol and buffered salt solution, ethanol and deionized water, ethanol and PBS, or ethanol buffered salt solution. The first thiol-based small molecule may be present in the first solvent at a concentration of between about 0.1 mM and about 50 mM, between about 0.5 mM and about 40 mM, between about 1 mM and about 30 mM, between about 2 mM and about 20 mM, or between about 5 mM and about 15 mM. In some variations, the electrode may be submerged in the first solution for a pre-determined period of time. The pre-determined time period may be between about 4 hours and about 48 hours, between about 12 hours and about 24 hours, or between about 16 hours and about 20 hours. In some variations, depositing the first thiol-based small molecule can be performed at a pre-determined temperature. The pre-determined temperature may be adjusted to control packing density of the first thiol-based small molecule. Decreasing the temperature may increase the packing density. The pre-determined temperature may be between about −20° C. and about 37° C., between about 1° C. and about 30° C., or between about 3° C. and about 23° C.

Following deposition of the first thiol-based small molecule at step 1830A, a second, different thiol-based small molecule may be deposited (i.e., applied) on the biorecognition layer at step 1840A of method 1810A. The second, different thiol-based small molecule may be any one of the thiol-based small molecules described herein and may be deposited according to any suitable method, including but not limited to drop casting, printing, spray coating, soaking, spin coating, and chemical deposition. For instance, the second, different thiol-based small molecule may be deposited by soaking, or submerging, the electrode in a second solution. In some variations, the second solution may comprise the second, different thiol-based small molecule in a second solvent. The second solvent may comprise an organic solvent or an inorganic solvent. For instance, the second solvent may be one or more solvents selected from the group consisting of: ethanol, methanol, propanol, isopropanol, butanol, acetone, acetonitrile, dimethylsulfoxide, dimethylformamide, dimethylacetamide, propylene carbonate, ethylene carbonate, deionized water, PBS, and a buffered salt solution. In an example, the second solvent may be ethanol. In an example, the second solvent may be a mixture of methanol or ethanol and deionized water, PBS, or a buffered salt solution. The second, different thiol-based small molecule may be present in the second solvent at a concentration of between about 0.1 mM and about 50 mM, between about 0.5 mM and about 40 mM, between about 1 mM and about 30 mM, between about 2 mM and about 20 mM, or between about 5 mM and about 15 mM. In some variations, the electrode may be submerged in the second solution for a pre-determined period of time. The pre-determined time period may be between about 4 hours and about 48 hours, between about 12 hours and about 24 hours, or between about 16 hours and about 20 hours. In some variations, depositing the second, different thiol-based small molecule can be performed at a pre-determined temperature. The pre-determined temperature may be adjusted to control packing density of the second, different thiol-based small molecule. Decreasing the temperature may increase the packing density. The pre-determined temperature may be between about −20° C. and about 37° C., between about 1° C. and about 30° C., or between about 3° C. and about 23° C.

In some variations, subsequent, different thiol-based small molecules can be deposited at step 1850A of method 1810A. Any number of thiol-based small molecules can be deployed in order to achieve the passivating qualities desired.

Referring now to FIG. 18B, a variation of method 1810A will be described wherein each of a first thiol-based small molecule and a second, different thiol-based small molecule are deposited concurrently. Following deposition of the biorecognition element at step 1820B, as described above, the first thiol-based small molecule and the second, different thiol-based small molecule may be deposited at step 1860B of method 1810B according to any suitable method, including but not limited to drop casting, printing, spray coating, soaking, spin coating, and chemical deposition. For instance, the thiol-based small molecules may be deposited by soaking, or submerging, the electrode in a solution. In some variations, the solution may comprise the first thiol-based small molecule in a first solvent and the second, different thiol-based small molecule in a second solvent. The first solvent and the second solvent may comprise an organic solvent or an inorganic solvent. For instance, the first solvent and the second solvent may be one selected from the group consisting of ethanol, methanol, propanol, isopropanol, butanol, acetone, acetonitrile, dimethylsulfoxide, dimethylformamide, dimethylacetamide, propylene carbonate, ethylene carbonate, deionized water, phosphate buffered saline, and a buffered salt solution. In some variations, the first solvent and the second solvent may be the same. In other variations, the first solvent and the second solvent may be different.

In some variations, the first thiol-based small molecule may be present in the first solvent at a concentration of between about 0.1 mM and about 50 mM, between about 0.5 mM and about 40 mM, between about 1 mM and about 30 mM, between about 2 mM and about 20 mM, or between about 5 mM and about 15 mM. The second thiol-based small molecule may be present in the second solvent at a concentration of between about 0.1 mM and about 50 mM, between about 0.5 mM and about 40 mM, between about 1 mM and about 30 mM, between about 2 mM and about 20 mM, or between about 5 mM and about 15 mM.

In some variations, the electrode may be submerged in solution combining the first solution and the second solution for a pre-determined period of time. The pre-determined time period may be between about 4 hours and about 48 hours, between about 12 hours and about 24 hours, or between about 16 hours and about 20 hours. In some variations, the first thiol-based small molecule and the second, different thiol-based small molecule can be deposited at a pre-determined temperature. The pre-determined temperature may be between about −20° C. and about 37° C., between about 1° C. and about 30° C., or between about 3° C. and about 23° C.

In some variations, the combined solution of the first thiol-based small molecule and the second, different thiol-based small molecule comprises an organic solvent in water at a concentration of at least about 5% v/v, at least about 10% v/v, at least about 15% v/v, at least about 20% v/v, at least about 25% v/v, at least about 30% v/v, at least about 35% v/v, at least about 40% v/v, at least about 45% v/v, at least about 50% v/v, at least about 55% v/v, at least about 60% v/v, at least about 65% v/v, at least about 70% v/v, at least about 75% v/v, at least about 80% v/v, at least about 85% v/v, at least about 90% v/v, or at least about 95% v/v.

In some variations, the combined solution of the first thiol-based small molecule and the second, different thiol-based small molecule comprises the first thiol-based passivation molecule and the second, different thiol-based passivation molecule at a ratio of between about 1 mM and about 30 mM to between about 2.5 mM and about 30 mM. For instance, when the first thiol-based small molecule is MCH and the second, different thiol-based small molecule is PC, the combined solution comprises the first thiol-based passivation molecule and the second, different thiol-based passivation molecule at a ratio of between about 1 mM and about 30 mM MCH to between about 2.5 mM and about 30 mM PC. In an example, the combined solution comprises the first thiol-based passivation molecule and the second, different thiol-based passivation molecule at a ratio of MCH to PC of 15 mM MCH to 10 mM PC.

In some variations, the composition of the passivation element may be selected according to the desired functionality of the resulting device. For instance, it is shown herein that a sensor with only MCH as the passivation element has improved signal strength but reduced long term stability. It is also shown herein that a sensor with only PC as the passivation element has excellent long term stability but reduced signal strength. Accordingly, in some variations, it may be beneficial to utilize a passivation element comprising both MCH and PC. In these variations, the desired composition, or ratio of MCH to PC, depends upon the desired functional properties. The composition of the passivation element can be tailored to provide different sensor characteristics, such as, for example, different levels of long term stability, different levels of signal strength, and the like, depending upon the passivation molecules selected.

In some variations, the passivation element can be deposited until a predetermined density of the passivation element is achieved. In some variations, subsequent thiol-based small molecules can be deposited together with the first and second, different thiol-based small molecules or may be deposited in a subsequent step as shown in FIG. 18A. Any number of thiol-based small molecules can be deployed in order to achieve the passivating qualities desired.

In some variations, method 1810A and method 1810B may further comprise depositing zwitterions (e.g., zwitterionic peptides or thiol-based small molecules with a zwitterionic phosphorylcholine head group) as part of the passivation element.

In some variations, at least one of the first thiol-based passivation molecule and the second thiol-based passivation molecule within the solution have enhanced solubility. Enhanced solubility may be provided by the inclusion of surfactants and/or emulsifiers in the solution or by the use of passivation molecules having these properties. For instance, some of the above-described thiol-based small molecules may be sufficiently amphiphilic that they demonstrate the same properties as surfactants and/or emulsifiers. This is the case, for example, with MCH and PC.

In some variations, the passivation element may be deposited on a surface of the electrode material at concentration of between about 1 mM and about 30 mM, between about 1 mM and about 15 mM, between about 5 mM and about 20 mM, between about 5 mM and about 10 mM, about 2 mM, about 4 mM, about 5 mM, about 6 mM, about 7 mM, about 8 mM, about 10 mM, about 15 mM and about 20 mM. In some variations, a mixture of the biorecognition element and the passivation element may be applied onto the electrode material surfaces. The biorecognition element and passivation elements may be mixed at appropriate relative concentrations in order to achieve a desired surface density of the biorecognition elements. The appropriate relative concentrations may depend on the specific biorecognition elements and passivation elements being mixed, but the biorecognition element to passivation elements ratio may be between about 1:100 to about 1:10.

In some variations, the working electrode surface may be electrochemically roughened in order to enhance adhesion of the biorecognition layer to the electrode material 1612 surface (and/or Pt black layer). The roughening process may involve a cathodization treatment (e.g., cathodic deposition, a subset of amperometry) wherein the electrode is subject to a fixed cathodic potential (e.g., between −0.4 V and +0.2 V vs. Ag/AgCl reference electrode) for a certain amount of time (e.g., 5 sec to 10 min) in an acid solution containing the desired metal cation dissolved therein (e.g., 0.01 mM to 100 mM H₂PtCl₆). Alternatively, the electrode is subject to a fixed cathodic potential (e.g., between about −0.4 V to about +0.2 V vs. Ag/AgCl reference electrode) until a certain amount of charge has passed (e.g., 0.1 mC-100 mC) in an acid solution containing the desired metal cation dissolved therein (e.g., 0.01 mM to 100 mM H₂PtCl₆). In this process, a thin, yet highly porous layer of the metal may be generated on the electrode surface, thereby augmenting the electrode surface area dramatically. Additionally or alternatively, in some variations as described above, elemental platinum metal may be deposited on the electrode to form or deposit a platinum black layer 1613.

### Counter Electrode

Anodization: In some variations, the counter electrode material may undergo an anodization treatment using an amperometry approach in which the electrode constituent(s) assigned for the count electrode function is subject to a fixed high anodic potential or a suitable amount of time in a moderate-strength acid solution. Exemplary parameters and other specifics of the anodization process for the counter electrode may be similar to that described above for the working electrode. Similarly, anodization for the counter electrode may alternatively use a coulometry approach as described above.

Activation: In some variations, following the anodization process, the counter electrode constituents may be subjected to a cyclically-scanned potential waveform in an activation process using cyclic voltammetry. In some variations, the activation process may be similar to that described above for the working electrode.

Roughening: Furthermore, in some variations, the counter electrode surface may be electrochemically roughened in order to enhance the current-sinking or current-sourcing capacity of this electrode contingent. The electrochemical roughening process may be similar to that described above for the working electrode. Additionally or alternatively, in some variations as described above, elemental platinum metal may be deposited on the electrode to form or deposit a platinum black layer 1624.

### Reference Electrode

Anodization: Like the working and counter electrodes as described above, the reference electrode, in some variations, may undergo an anodization treatment using an amperometry approach in which the electrode constituent(s) assigned for the counter electrode function is subject to a fixed high anodic potential or a suitable amount of time in a moderate-strength acid solution. Exemplary parameters and other specifics of the anodization process for the counter electrode may be similar to that described above for the working electrode. Similarly, anodization for the counter electrode may

[symbol]

Activation: Following the anodization process, the reference electrode constituents may, in some variations, be subjected to a cyclically-scanned potential waveform in an activation process using cyclic voltammetry. In some variations, the activation process may be similar to that described above for the working electrode.

Functionalization: Following the activation process, the reference electrode constituents may, in some variations, be functionalized. In some variations, the reference electrode may be functionalized with silver plating, by way of example with AgNO3, followed by oxidation in chloride containing media, by way of example KCl. Assuming that the reference electrode contingent of the microneedle array has undergone the aforementioned steps, a fixed anodic potential (e.g., between +0.4-+1.0 V vs. Ag/AgCl reference electrode) may be applied for a certain suitable duration (e.g., between about 10 sec and about 10 min) in an aqueous solution. Alternatively, the reference electrode is subject to a fixed anodic potential (e.g., between about +0.4 V to about +1.0 V vs. Ag/AgCl reference electrode) until a certain amount of charge has passed (e.g., 0.01 mC-10 mC) in an aqueous solution. In some variations, the aqueous solution may include a monomeric precursor to a conducting polymer and a charged dopant counter ion or material (e.g., poly(styrene sulfonate)) carrying an opposing charge. In this process, a thin film (e.g., between about 10 nm and about 10,000 nm) of a conducting polymer with a dispersed counter ion or material may be generated on the reference electrode surface. This creates a surface-immobilized, solid-state redox coupled with a stable thermodynamic potential. In some variations, the conducting polymer may include one or more of aniline, pyrrole, acetylene, phenylene, phenylene vinylene, phenylene diamine, thiophene, 3,4-ethylenedioxythiophene, and aminophenylboronic acid.

In some alternative embodiments, a native iridium oxide film (e.g., IrO₂ or Ir₂O₃ or IrO₄) may be electrochemically grown on an iridium electrode surface in an oxidative process. This also creates a stable redox couple, as discussed above.

Furthermore, in some variations the reference electrode surface may be electrochemically roughened in order to enhance adhesion of the surface-immobilized redox couple. The electrochemical roughening process may be similar to that described above for the working electrode. Additionally or alternatively, in some variations as described above, elemental platinum metal may be deposited on the electrode to form or deposit a platinum black layer 1633.

### Sensor Warm-Up

Warm-up: Many implanted electrochemical sensors require a “warm-up” time, or time for the sensor to attain a stable signal value following implantation. This process has origins in both physiology and sensor dynamics. However, various aspects of analyte monitoring devices described herein are configured to mitigate factors contributing to warm-up time. For example, the analyte monitoring devices described herein may have a warm-up time of about 30 minutes or less (e.g., between about 10 minutes and about 30 minutes, between about 15 minutes and about 30 minutes, between about 20 minutes and about 30 minutes, between about 25 minutes and about 30 minutes), about 45 minutes or less, about 60 minutes or less, about 90 minutes or less, or about 120 minutes or less. In some variations, following a warm-up period, the analyte monitoring device may calibrate during a calibration period.

Wound response: For example, the implantation of a sensor creates a wound response due to the localization disruption, displacement, and destruction of tissue. The larger the sensor, or the deeper the implant, the more prolific the wound response. Accordingly, there is a compelling rationale to miniaturize the sensor as well as cover one or more electrodes (and/or a sensor, sensor array, microneedle, microneedle array, or device comprising the electrodes) with a biocompatible layer to elicit an attenuated wound response, which would result in a more rapid warm-up.

Protein adsorption: Additionally, following implantation of a sensor, the foreign body response is immediately instigated. The foreign body response includes a complex biochemical cascade that aims to encapsulate the foreign material with cellular matter. Hydrophobic surfaces tend to be subject to adsorption of endogenous proteins very rapidly following implant; this is referred to as biofouling. Hydrophilic surfaces, on the other hand, resist biofouling due to high water content. Human serum albumin (HSA) is the predominant protein in the dermal interstitial fluid, constituting about 60% of total protein, and maintains a negative charge at physiological pH. When the sensor is polarized with a positive potential (as in some variation of the analyte monitoring device), endogenous HSA is subject to electric drift and charge attraction to the positive (working) electrode of the sensor. This can give rise to an increased propensity for the sensor surface to become biofouled. This is a rationale behind the implementation of a biocompatible layer to effectively conceal the sensor from being recognized as a foreign body, as described in further detail above.

As described herein, the analyte monitoring device reduces the influence of the above physiological factors on warm-up time due to, for example, the shallow nature of the implant, the minimal volume of tissue displaced, the minimal amount of trauma to said tissue during implantation, and the lack of permeation of the vasculature deeper in the reticular dermis, which, when perturbed, can instigate a more prolific wound response that will engender an accelerated effort to encapsulate the implant.

Attainment of equilibrium: One example of the effect of sensor dynamics on warm-up time relates to the attainment of equilibrium. An electrochemical sensor requires a finite amount of time to achieve equilibrium when used in a new environment. This is typically associated with the establishment of thermodynamic equilibrium due to an adsorbed surface layer of ions at the electrodes. As the reference electrode in most implantable electrochemical sensors does not employ an internal filling solution with a redox couple that is sealed from the rest of the electrochemical cell, this reference electrode must attain equilibrium with its surroundings in order to establish a stable reference potential.

Hydration of sensor layers: The electrode sensor layers must be immersed in an aqueous environment to function properly. The resulting hydration process may activate the electrode's polymer layer(s) such as the biocompatible layer, as well as biorecognition element(s), linkers, and/or passivation element(s) in the biorecognition layer, and allows them to rearrange and return to their native active tertiary structure, which is primarily responsible for their activity or unique properties. This process is often known as sensor ‘wetting’ and allows the medium in which the sensing operation occurs to intercalate the sensor layers to a sufficient extent. Thus, a rationale behind the implementation of a biocompatible layer is to minimize or mitigate the degree of rearrangement and return to the native tertiary structures of one or more components of the biorecognition layers during the hydration process.

### Electronics System

As shown in the schematic of FIG. 2A of an analyte monitoring device 110, the electronics system 120 may be integrated within the housing 112, such that the electronics system 120 may be combined with sensing elements (e.g., microneedle array) as part of a single unit. Further details of an example variation of an electronics system 120 are described below.

### Analog Front End

In some variations, the electronics system of the analyte monitoring device may include an analog front end. The analog front end may include sensor circuitry (e.g., sensor circuitry 124 as shown in FIG. 2A) that converts analog current measurements to digital values that can be processed by the microcontroller. The analog front end may, for example, include a programmable analog front end that is suitable for use with electrochemical sensors. For example, the analog front end may include a MAX30131, MAX30132, or MAX30134 component (which have 1, 2, and 4 channel, respectively), available from Maxim Integrated (San Jose, CA), which are ultra-low power programmable analog front ends for use with electrochemical sensors. The analog front end may include an EmStat® Pico Module or an EmStat® Pico Core from PalmSens (Houten, Netherlands) for running SWV. The analog front end may also include an AD5940, ADuCM355 or AD5941 component, available from Analog Devices (Norwood, MA), which are high precision, impedance and electrochemical front ends. Similarly, the analog front end may also include an LMP91000, available from Texas Instruments (Dallas, TX), which is a configurable analog front end potentiostat for low-power chemical sensing applications. The analog front end may provide biasing and a complete measurement path, including the analog to digital converters (ADCs). Ultra-low power may allow for the continuous biasing of the sensor to maintain accuracy and fast response when measurement is required for an extended duration (e.g. 7 days) using a body-worn, battery-operated device.

In some variations, the analog front-end device may be compatible with both two and three terminal electrochemical sensors, such as to enable both DC current measurement, AC current measurement, and electrochemical impedance spectroscopy (EIS) measurement capabilities. Furthermore, the analog front end may include an internal temperature sensor and programmable voltage reference, support external temperature monitoring and an external reference source and integrate voltage monitoring of bias and supply voltages for safety and compliance.

In some variations, the analog front end may include a multi-channel potentiostat to multiplex sensor inputs and handle multiple signal channels.

In some variations, the analog front end and peripheral electronics may be integrated into an application-specific integrated circuit (ASIC), which may help reduce cost, for example. This integrated solution may include the microcontroller described below, in some variations.

### Microcontroller

In some variations, the electronics system of the analyte monitoring device may include at least one microcontroller (e.g., controller 122 as shown in FIG. 2A). The microcontroller may include, for example, a processor with integrated flash memory. In some variations, the microcontroller in the analyte monitoring device may be configured to perform analysis to correlate sensor signals to an analyte measurement. For example, the microcontroller may execute a programmed routine in firmware to interpret the digital signal (e.g., from the analog front end), perform any relevant algorithms and/or other analysis, and route processed data to and/or from the communication module. Keeping the analysis on-board the analyte monitoring device may, for example, enable the analyte monitoring device to broadcast analyte measurement(s) to multiple devices (e.g., mobile computing devices such as a smartphone or smartwatch, therapeutic delivery systems such as insulin pens or pumps, etc.) in parallel, while ensuring that each connected device has the same information.

In some variations, the microcontroller may be configured to activate and/or inactivate the analyte monitoring device on one or more detected conditions. For example, the device may be configured to power on the analyte monitoring device upon insertion of the microneedle array into skin. This may, for example, enable a power-saving feature in which the battery is disconnected until the microneedle array is placed in skin, at which time the device may begin broadcasting sensor data. Such a feature may, for example, help improve the shelf life of the analyte monitoring device and/or simplify the analyte monitoring device-external device pairing process for the user.

Additionally or alternatively, the microcontroller may be configured to actively confirm the insertion of the microneedle array into skin based on sensor measurements performed with the microneedle array. For example, after two or more microneedles in the microneedle array are presumed to have been inserted into skin, a fixed or time-varying electrical potential or current may be applied to those microneedles. A measurement result (e.g., electrical potential or current value) of a signal generated between the electrodes of the inserted microneedles is measured, and then compared to a known reference value to corroborate successful insertion of the microneedle array into the skin. The reference value may, for example, include a voltage, a current, a resistant, a conductance, a capacitance, an inductance and/or an impedance.

In some variations, the microcontroller may utilize an 8-bit, 16-bit, 32-bit, or 64-bit data structure. Suitable microcontroller architectures include ARM® and RISC® architectures, and flash memory may be embedded or external to the microcontroller for suitable data storage. In some variations the microcontroller may be a single core microcontroller, while in some variations the microcontroller may be a multi-core (e.g., dual core) microcontroller which may enable flexible architectures for optimizing power and/or performance within the system. For example, the cores in the microcontroller may include similar or differing architectures. For example, in an example variation, the microcontroller may be a dual core microcontroller including a first core with a high performance and high-power architecture, and a second core with a low performance and low power architecture. The first core may function as a “workhorse” in that it may be used to process higher performance functions (e.g., sensor measurements, algorithmic calculations, etc.), while the second core may be used to perform lower performance functions (e.g., background routines, data transmission, etc.). Accordingly, the different cores of the microcontroller may be run at different duty cycles (e.g., the second core for lower performance functions may be run at a higher duty cycles) optimized for their respective functions, thereby improving overall power efficiency. Additionally or alternatively, in some variations the microcontroller may include embedded analog circuitry, such as for interfacing with additional sensor(s) and/or the microneedle array. In some variations, the microcontroller may be configured to operate using a 0.8V-5V power source, such as a 1.2V-3V power source.

### Methods of Manufacturing and Drying Biocompatible Layer

As shown by way of example in FIGS. 16A-16M, the biorecognition layer 1614 may have a relatively complex molecular structure with various macromolecular components, such as aptamers, arranged in an ordered fashion. As such, the layer is susceptible to degradation and damage. Moreover, the biorecognition layer 1614 may be configured to perform its function in an aqueous environment, and exposure to air, for example during dry storage, may accelerate degradation due to removal of the component molecules from an aqueous environment. It was found that, unexpectedly and advantageously, a partially or fully desiccated hydrogel biocompatible layer 1616 disposed on the biorecognition layer 1614 protects the biorecognition layer 1614 from degradation during dry storage.

### Methods of Applying a Hydrogel Biocompatible Layer

In some variations, the biocompatible layer 1616 may be applied over the biorecognition layer 1614 by means of at least one of spray coating, dip coating, drop casting, chemical vapor deposition, plasma vapor deposition, and electro-deposition. In some variations, in which a hydrogel biocompatible layer is by way of example applied by drop casting or spray coating, the hydrogel may be drop casted on the biorecognition layer a temperature of between about 45° C. and about 75° C.

### Methods of Drying the Hydrogel Biocompatible Layer

A working electrode (e.g. working electrode 1610A) comprising a biorecognition layer (e.g. biorecognition layer 1614) may be prepared for dry storage by applying a hydrogel biocompatible layer (e.g. biocompatible layer 1616) on the biorecognition layer, and drying the hydrogel to a desired level of desiccation (for example, to an at least partially desiccated state). The hydrogel biocompatible layer may be applied using a variety of deposition techniques and may be dried under various conditions such as, for example, a variety of temperatures and relative humidities, to achieve a variety of desiccation levels.

The hydrogel, once applied, may be dried under controlled conditions until the hydrogel achieves a desired level of desiccation. In some variations, the hydrogel may be dried at an ambient temperature of between about 15 degC and about 30 degC, and an ambient humidity of between about 10% and about 80% relative humidity. In some variations, the ambient temperature may between about 15 degC and about 25 degC, between about 18 degC and about 22 degC, between about 15 degC and about 20 degC, or at room temperature. In some variations, the ambient humidity may between about 50% and about 80% relative humidity, between about 70% and about 80% relative humidity, between about 20% and about 60% relative humidity, between about 30% and about 70% relative humidity, about 50% relative humidity, about 60% relative humidity, about 70% relative humidity and about 80% relative humidity.

As mentioned above, the hydrogel may be dried under controlled conditions until that the hydrogel achieves a desired level of desiccation. In some variations, the hydrogel may be dried until the at least partially desiccated hydrogel may comprise about 10%, about 20%, about 30%, about 40%, about 50%, about 60%, about 70%, about 80%, or about 90% of water compared to a fully hydrated state of the hydrogel. In some variations, the at least partially desiccated hydrogel may be fully desiccated.

In some variation, after the drying step, one or more electrodes (e.g., the working electrode and/or a sensor, sensor array, microneedle, microneedle array, or device comprising the working electrode) may be stored in exposure to air. The duration of time of the storage may be between about 1 day and about 1 week, between about 1 day and 1 month, between about 3 days and 1 week, about 1 day, about 2 days, about three days, about 4 days, about 5 days, about 6 days, or about 7 days. In a case where the hydrogel biocompatible layer is disposed on the biorecognition layer, the rate biorecognition layer degrades at a rate of between about 1% per day and about 5% per day, not more than about 3% per day, not more than about 2% per day, not more than about 1% per day, and not more than about 5% per day. The storage condition may be or both of an ambient temperature of between about 15 degC and about 30 degC, and an ambient humidity of between about 10% and about 80% relative humidity, between about 10% and about 50% relative humidity between about 30% and about 80% relative humidity, or between about 20% and about 50% relative humidity.

In some variations, after the drying step, the one or more electrodes (e.g., the working electrode and/or a sensor, sensor array, microneedle, microneedle array, or device comprising the working electrode) may be packaged for storage. For storage, the one or more electrodes (e.g., the working electrode and/or a sensor, sensor array, microneedle, microneedle array, or device comprising the working electrode) may be packaged in a sealed disposable package, wherein the interior of the package has an ambient humidity between about 50% and about 80% relative humidity. In some variations, the interior of the package may have about the same ambient humidity as the drying step. In some variations, the interior of the package may have about the same ambient humidity that is about 10% higher, 20% higher, or 30% higher than the ambient humidity of the drying step. The interior humidity may be controlled or maintained in a particular range with the inclusion of silica packets.

Protection of Biorecognition from Degradation During Sterilization with Hydrogel Biorecognition Layer

As described in detail herein, one or more electrodes (e.g., the working electrode 1610A, counter electrode 1620A, reference 1630A as show in FIGS. 16A-16C) may be used to monitor an analyte in a physiological fluid in a subject, and thus may be placed inside the subject, by way of example, in viable epidermis or dermis. As such, the one or more electrodes (e.g., the working electrode and/or a sensor, sensor array, microneedle, microneedle array, or device comprising the working electrode) may be sterilized prior to storage and/or use. During sterilization, the one or more electrodes (e.g., the working electrode and/or a sensor, sensor array, microneedle, microneedle array, or device comprising the working electrode) may be exposed to radiation. However, as shown by way of example in FIGS. 6A-6D, the biorecognition layer of the working electrode has a relatively complex molecular structure with various macromolecules such as aptamers arranged in an ordered fashion. Radiation exposure is well known to modify or degrade macromolecules and arrangements thereof (for example but not limited to small molecule thiols in the biorecognition layer). As such, the biorecognition layer may be susceptible to degradation and damage through radiation exposure. As shown in Example 2 herein below, presence of a hydrogel biocompatible layer unexpectedly and advantageously reduces degradation of the biorecognition layer due to radiation exposure.

In some variations, the working electrode (and/or a sensor, sensor array, microneedle, microneedle array, or device comprising the working electrode) comprising a biorecognition layer and a hydrogel biocompatible layer applied over said biorecognition layer may be sterilized with exposure to a radiation. The present disclosure provides for methods of sterilizing a sensor configured to generate a signal that is indicative of a concentration of an analyte in a fluid. The method may include the steps of: providing a working electrode comprising a biorecognition layer disposed on an electrode material, applying a hydrogel biocompatible layer on the biorecognition layer, and sterilizing the working electrode with exposure to a radiation.

The radiation may comprise ultraviolet radiation, gamma radiation, X-ray radiation, or electron beam radiation (E-Beam). In some variations in which E-Beam radiation is used, the E-Beam radiation may be applied to the working electrode (and/or a sensor, sensor array, microneedle, microneedle array, or device comprising the working electrode) at a dose of between about 2 megarads and about 4 megarads, between about 2.5 megarads and about 3.5 megarads, or between about 2.7 megarads and about 3.1 megarads.

As mentioned above, the biocompatible layer may protect the biorecognition layer from degradation, such as, for example, from radiation exposure during sterilization. In some variations, the biorecognition layer, when provided with the hydrogel biocompatible layer 1616 disposed over the biorecognition layer, may be degraded by not more than about 2%, not more than about 5%, not more than about 10%, not more than about 15%, or not more than about 20% after radiation exposure (e.g., during sterilization). The degradation of the biorecognition layer may be determined by placing the working electrode in a fluid comprising an analyte, gathering a signal from the working electrode indicative of a concentration of the analyte in the fluid, and comparing the strength of the signal to a reference signal strength. In some variations, the reference signal strength may be based on an initial signal measured by the same or equivalent working electrode within a certain time after being produced (which may be when the hydrogel is applied to the working electrode), that has not been sterilized, and placed in an equivalent measuring environment, for example in the same fluid. The certain period of time may be between about 1 hour and about 10 hours, between about 1 hour and about 5 hours, about 1 hour, about 2 hours, about 4 hours, about 5 hours and about 10 hours. In some variations, the reference signal strength may be based on a comparative signal measured by an equivalent working electrode stored for an equivalent time period and placed in an equivalent fluid, wherein the equivalent sensor lacks a hydrogel disposed on the biorecognition layer.

In some variations, the degradation of the biorecognition layer may be detected or quantified as loss of passivation. The loss of passivation may be detected as a change in one or more electrical or electrochemical properties, such as one or more square wave voltammetry properties, of the working electrode to more closely resemble a bare electrode surface with little or no passivation elements.

### Use of Analyte Monitoring System

Described below is an overview of various aspects of a method of use and operation of the analyte monitoring system, including the analyte monitoring device and peripheral devices, etc.

### Application of Analyte Monitoring Device

As described above, the analyte monitoring device is applied to the skin of a user such that the microneedle array in the device penetrates the skin and the microneedle array's electrodes are positioned in the upper dermis for access to dermal interstitial fluid. For example, in some variations, the microneedle array may be geometrically configured to penetrate the outer layer of the skin, the stratum corneum, bore through the epidermis, and come to rest within the papillary or upper reticular dermis. The sensing region, confined to the electrode at the distal extent of each microneedle constituent of the array (as described above) may be configured to rest and remain seated in the papillary or upper reticular dermis following application in order to ensure adequate exposure to circulating dermal interstitial fluid (ISF) without the risk of bleeding or undue influence with nerve endings.

In some variations, the analyte monitoring device may include a wearable housing or patch with an adhesive layer configured to adhere to the skin and fix the microneedle array in position. While the analyte monitoring device may be applied manually (e.g., removing a protective film on the adhesive layer, and manually pressing the patch onto the skin on a desired wear site), in some variations the analyte monitoring device may be applied to the skin using a suitable applicator.

The analyte monitoring device may be applied in any suitable location, though in some variations it may be desirable to avoid anatomical areas of thick or calloused skin (e.g., palmar and plantar regions), or areas undergoing significant flexion (e.g., olecranon or patella). Suitable wear sites may include, for example, on the arm (e.g., upper arm, lower arm), shoulder (e.g., over the deltoid), back of hands, neck, face, scalp, torso (e.g., on the back such as in the thoracic region, lumbar region, sacral region, etc. or on the chest or abdomen), buttocks, legs (e.g., upper legs, lower legs, etc.), and/or top of feet, etc.

As described above, in some variations the analyte monitoring device may be configured to automatically activate upon insertion, and/or confirm correct insertion into skin. Details of these features are described in further detail above.

### Pairing to Peripheral Device

In some variations, the analyte monitoring device may be paired to at least one peripheral device such that the peripheral device receives broadcasted or otherwise transmitted data from the analyte monitoring device, including measurement data. Suitable peripheral devices include, for example a mobile computing device (e.g., smartphone, smartwatch) which may be executing a mobile application. Additionally alternatively, an analyte monitoring device may be paired (or otherwise combined) with a therapeutic delivery device.

As described above, the pairing may be accomplished through suitable wireless communication modules (e.g., implementing Bluetooth). In some variations, the pairing may occur after the analyte monitoring device is applied and inserted into the skin of a user (e.g., after the analyte monitoring device is activated). Additionally or alternatively, the pairing may occur prior to the analyte monitoring device being applied and inserted into the skin of a user.

Thus, the paired mobile or other device may receive the broadcasted or transmitted data from the analyte monitoring device. The peripheral device may display, store, and/or transmit the measurement data to the user and/or healthcare provider and/or support network. Furthermore, in some variations, the said paired mobile or wearable device performs algorithmic treatment to the data to improve the signal fidelity, accuracy, and/or calibration, etc. In some variations, measurement data and/or other user info may additionally or alternatively be communicated and/or stored via network (e.g., cloud network).

By way of illustration, in some variations a mobile computing device or other computing device (e.g., smartphones, smartwatches, tablets, etc.) may be configured to execute a mobile application that provides an interface to display estimated analyte-based values, trend information and historical data, etc.

In some variations, the mobile application may use the mobile computing device's Bluetooth framework to scan for the analyte monitoring device. As shown in FIG. 20, the analyte monitoring device may power on or initialize as soon as it is applied to the skin, and the analyte monitoring device may begin the advertising process. The mobile application may then connect to the analyte monitoring device and begin priming the sensor for measurement. In case the mobile application detects multiple analyte monitoring devices, the mobile application may detect the analyte monitoring device that is closest in proximity to itself and/or may request the user (e.g., via the user interface on the mobile device) to confirm disambiguation. In some variations, the mobile application may also be capable of connecting to multiple analyte monitoring devices simultaneously. This may be useful, for example, to replace sensors that are reaching the end of their lifetime.

In some variations, the Bluetooth® Low Energy™ (BLE) protocol may be used for connectivity. For example, the sensor implements a custom BLE peripheral profile for the analyte monitoring system. Data may be exchanged after establishing a standard secure BLE connection between the analyte monitoring device and the smartphone, smartwatch, or tablet running the mobile application. The BLE connection may be maintained permanently for the life of the sensor. If the connection is broken due to any reasons (e.g., weak signal) the analyte monitoring device may start advertising itself again, and the mobile application may re-establish the connection at the earliest opportunity, for example, when in range based on physical proximity.

In some variations, there may be one or more additional layers of security implemented on top of the BLE connection to ensure authorized access consisting of a combination of one or more techniques such as passcode-protection, shared-secrets, encryption and multi-factor authentication.

The mobile application may guide the user through initiating a new analyte monitoring device. Once this process completes, the mobile application is not required for the analyte monitoring device to operate and record measurements. A secondary display device like a smartwatch can be authorized from the mobile application to receive analyte readings from the sensor directly.

Furthermore, in some variations the mobile application may additionally or alternatively help calibrate the analyte monitoring device. For example, the analyte monitoring device may indicate a request for calibration to the mobile application, and the mobile application may request calibration input from the user to calibrate the sensor.

### Sensor Measurements

Once the analyte monitoring device is inserted and warm-up and any calibration has completed, the analyte monitoring device may be ready for providing sensor measurements of analyte. The analyte from the biological milieu, through the biocompatible and diffusion-limiting layers on the working electrode, and to the biorecognition layer including the biorecognition element.

In some embodiments, a bias potential may be applied between the working and reference electrodes of the analyte monitoring device, and an electrical current may flow from the counter electrode to maintain the fixed potential relationship between the working and reference electrodes. This causes the oxidation or reduction of the electroactive product, causing a current to flow between the working electrodes and counter electrodes. The current value is proportional to the proximity of the redox reporter molecule functionalized to the analyte-binding aptamers to electrode material of the working electrode and, specifically, to the concentration of analyte in the dermal interstitial fluid according to the Cottrell relation, or some derivative thereof, as described in further detail above.

The electrical current may be converted to a voltage signal by a transimpedance amplifier and quantized to a digital bitstream by means of an analog-to-digital converter (ADC). Alternatively, the electrical current may be directly quantized to a digital bitstream by means of a current-mode ADC. The digital representation of the electrical current may be processed in the embedded microcontroller(s) in the analyte monitoring device and relayed to the wireless communication module for broadcast or transmission (e.g., to one or more peripheral devices). In some variations, the microcontroller may perform additional algorithmic treatment to the data to improve the signal fidelity, accuracy, and/or calibration, etc.

In some variations, the digital representation of the electrical current, or sensor signal, may be correlated to analyte measurement by the analyte monitoring device. For example, the microcontroller may execute a programmed routine in firmware to interpret the digital signal and perform any relevant algorithms and/or other analysis. The interpretation of the digital signal may include conversion of the digital signal into user status based on analyte measurements that is relevant to a user or a care provider. Examples of user status include: analyte concentration in a bodily fluid, by way of example dermal interstitial fluid or blood; a % change in analyte concentration; whether or not the analyte concentration is above, within, or below a threshold; and a psychological state of the user, by way of example a degree of stress and/or whether the stress is acute or chronic or to track diurnal variation in analyte levels. Keeping the analysis on-board the analyte monitoring device may, for example, enable the analyte monitoring device to broadcast analyte measurement(s) to multiple devices in parallel, while ensuring that each connected device has the same information. Thus, generally, the user's analyte-based values may be estimated and stored in the analyte monitoring device and communicated to one or more peripheral devices.

Data exchange can be initiated by either the mobile application or by the analyte monitoring device. For example, the analyte monitoring device may notify the mobile application of new analyte data as it becomes available. The frequency of updates may vary, for example, between about 5 seconds and about 5 minutes, and may depend on the type of data. Additionally or alternatively, the mobile application may request data from the analyte monitoring device (e.g., if the mobile application identifies gaps in the data it has collected, such as due to disconnections).

If the mobile application is not connected to the analyte monitoring device, the mobile application may not receive data from the sensor electronics. However, the electronics in the analyte monitoring device may store each actual and/or estimated analyte data point. When the mobile application is reconnected to the analyte monitoring device, it may request data that it has missed during the period of disconnection and the electronics on the analyte monitoring device may transmit that set of data as well (e.g., backfill).

Generally, the mobile application may be configured to provide display of real-time or near real-time analyte measurement data, such as on the display of the mobile computing device executing the mobile application. In some variations, the mobile application may communicate through a user interface regarding analysis of the analyte measurement, such as alerts, alarms, insights on trends, etc. such as to notify the user of analyte measurements requiring attention or follow-up action (e.g., high analyte measurements, low analyte measurements, high rates of change, analyte measurements outside of a pre-set range, etc.). In some variations, the mobile application may additionally or alternatively facilitate communication of the measurement data to the cloud for storage and/or archive for later retrieval.

### Interpreting Analyte Monitoring Device User Interface

In some variations, information relating to analyte measurement data and/or the analyte monitoring device may be communicated via a user interface of the analyte monitoring device. In some variations, the user interface of the analyte monitoring device may be used to communicate information to a user in addition to, or as an alternative to, communicating such information via a peripheral device such as through a mobile application on a computing device. Accordingly, a user and/or those around the user may easily and intuitively view the analyte monitoring device itself for an assessment of analyte measurement data (e.g., analyte measurement status such as current and/or trending analyte measurement levels) and/or device status, without the need to view a separate device (e.g., peripheral device or other device remote from, and in communication with, the analyte monitoring device). Availability of such information directly on the analyte monitoring device itself may also enable a user and/or those around the user to more promptly be alerted of any concerns (e.g., analyte measurements that are above or below target range, and/or analyte measurements that are increasing or decreasing at an alarming rate), thereby enabling a user to take appropriate corrective action more quickly.

### Numbered Embodiments of the Invention

Notwithstanding the appended claims, the disclosure sets forth the following numbered embodiments:

Embodiment I-1. A working electrode, comprising:
an electrode material;
a biorecognition element disposed on the electrode material and configured to selectively and reversibly bind to an analyte in a fluid;
a first thiol-based passivation molecule disposed on the electrode material; and
a second, different thiol-based passivation molecule disposed on the electrode material.

Embodiment I-2. The working electrode of embodiment I-1, wherein the first thiol-based passivation molecule and the second, different thiol-based passivation molecule are selected from the group consisting of: 1-hexanethiol, 6-mercapto-1-hexanamine, 6-mercapto-1-phosphatidylcholine hexane, 6-mercapto-1-hexanol (MCH), 7-mercapto-1-heptanol, 8-mercapto-1-octanol (MCO), 9-mercapto-1-nonanol, 10-mercapto-1-decanol, 11-mercapto-1-undecanol, 6-amino-1-hexanethiol, 7-amino-1-heptanethiol, 8-amino-1-octanethiol, 9-amino-1-nonanethiol, 10-amino-1-decanethiol, 11-amino-1-undecanethiol, (6-mercaptohexyl)-N,N,N-trimethylammonium bromide, (7-mercaptoheptyl)-N,N,N-trimethylammonium bromide, (8-mercaptooctyl)-N,N,N-trimethylammonium bromide, (9-mercaptononyl)-N,N,N-trimethylammonium bromide, (10-mercaptodecyl)-N,N,N-trimethylammonium bromide, (11-mercaptoundecyl)-N,N,N-trimethylammonium bromide, 6-mercaptohexylphosphoric acid, 7-mercaptoheptylphosphoric acid, 8-mercaptooctylphosphoric acid, 9-mercaptononylphosphoric acid, 10-mercaptodecylphosphoric acid, 11-mercaptoundecylphosphoric acid, 6-mercaptohexanoic acid, 7-mercaptoheptanoic acid, 8-mercaptooctanoic acid, 9-mercaptononanoic acid, 10-mercaptodecanoic acid, 11-mercaptoundecanoic acid, 6-mercaptohexanesulfonate, 7-mercaptoheptanesulfonate, 8-mercaptooctanesulfonate, 9-mercaptononanesulfonate, 10-mercaptodecanesulfonate, 11-mercaptoundecanesulfonate, poly(ethylene glycol) dithiol, 1,6-hexanedithiol, 1,7-heptanedithiol, 1,8-octanedithiol, 1,9-nonanedithiol, 1,10-decanedithiol, 1,11-undecanedithiol, 2-((3-((6-mercaptohexyl)thio)-2-methylpropanoyl)oxy)ethyl (2-trimethylammonium)ethyl)phosphate (PC), thiolated oligoethylene glycol (OEG), thiolated polyethylene glycol, (3-((6-mercaptohexyl)thio)-2-methylpropanoate-(2-ethyltrimethylammonium) chloride (AC), (3-((6-mercaptohexyl)thio)-2-methylpropanoate-(2-ethylsulfonate) potassium (SP), and (3-((6-mercaptohexyl)thio)-2-methylpropanoate-(2-ethyldimethylammonio-(3-propanesulfonate)) (AP), 1,2-bis-(11-sulfanyloundecanoyl)-sn-glycero-3-phosphocholine, methacrylate alkyl thiol ester, methacrylate PEG thiol ester, acrylate alkyl thiol ester, acrylate PEG thiol ester, vinyl terminated alkyl thiol, vinyl terminated PEG thiol, acetylene terminated alkyl thiol, acetylene terminated PEG thiol, benzophenone terminated alkyl thiol, benzophenone terminated PEG thiol, azide terminated alkyl thiol, azide terminated PEG thiol, N-hydroxysuccinimide terminated alkyl thiol, N-hydroxysuccinimide terminated PEG thiol, ferrocene terminated alkyl thiol, ferrocene terminated PEG thiol, methylene blue terminated alkyl thiol, methylene blue terminated PEG thiol, anthraquinone terminated alkyl thiol, anthraquinone terminated PEG thiol, hydroquinone terminated alkyl thiol, hydroquinone terminated PEG thiol, RGD peptide terminated thiol, and YIGSR peptide terminated thiol.

Embodiment I-3. The working electrode of embodiment I-2, wherein the first thiol-based passivation molecule is MCH and the second, different thiol-based passivation molecule is PC.

Embodiment I-4. The working electrode of embodiment I-1, wherein the working electrode comprises a third, different thiol-based passivation molecule.

Embodiment I-5. The working electrode of embodiment I-1, wherein each of the first thiol-based passivation molecule and the second, different thiol-based passivation molecule comprise a plurality of passivation molecules.

Embodiment I-6. The working electrode of embodiment I-5, wherein a distal end of the first thiol-based passivation molecule, the second, different thiol-based passivation molecule, or both, comprises one or more of a hydrophilic moiety, a hydrophobic moiety, a charged moiety, and a zwitterionic moiety.

Embodiment I-7. The working electrode of embodiment I-6, wherein the distal end of the first thiol-based passivation molecule, the second, different thiol-based passivation molecule, or both, comprises a zwitterionic moiety and the zwitterionic moiety is a zwitterionic phosphorylcholine head group.

Embodiment I-8. The working electrode of embodiment I-5, wherein the first thiol-based passivation molecule, the second, different thiol-based passivation molecule, or both, is a zwitterionic peptide.

Embodiment I-9. The working electrode of embodiment I-8, wherein the zwitterionic peptide comprises a cysteine residue.

Embodiment I-10. The working electrode of embodiment I-9, wherein the zwitterionic peptide comprises a thiol group as a side chain of the cysteine residue.

Embodiment I-11. The working electrode of embodiment I-1, wherein the electrode material comprises platinum, silver, palladium, iridium, rhodium, gold, ruthenium, titanium, nickel, carbon, glassy carbon, pyrolytic carbon, doped diamond, boron-doped diamond, or combinations thereof.

Embodiment I-12. The working electrode of embodiment I-1, wherein a pseudo-film formed by the first thiol-based passivation molecule and the second, different thiol-based passivation molecule on the electrode material has a thickness of between about 0.001 nm and about 1000 nm, between about 0.01 nm and about 100 nm, between about 0.1 nm and about 10 nm, between about 0.05 nm and about 500 nm, between about 0.1 nm and about 200 nm, between about 1 nm and about 1000 nm, between about 1 μm and about 500 μm, between about 2 μm and about 400 μm, between about 3 μm and about 300 μm, between about 4 μm and about 200 μm, between about 5 μm and about 100 μm, between about 10 μm and about 50 μm, or between about 20 μm and about 40 μm.

Embodiment I-13. The working electrode of embodiment I-1, wherein the biorecognition element is functionalized with a redox-active molecule and is configured to change conformation upon binding the analyte to move the redox-active molecule closer to or further from the electrode material.

Embodiment I-14. The working electrode of embodiment I-13, wherein the redox-active molecule comprises one selected from the group consisting of: methylene blue, ferrocene, pentamethyl ferrocene, C5-ferrocene, Nile blue, thionine, anthraquinone, hydroquinone, C5-anthraquinone, gallocyanine, indophenol, neutral red, dabcyl, carboxy-X-rhodamine, and π extended tetrathiafulvalene (exTTF).

Embodiment I-15. The working electrode of embodiment I-13, wherein the biorecognition element is an aptamer.

Embodiment I-16. The working electrode of embodiment I-15, wherein the aptamer comprises a thiol group at 3′ or 5′ terminal.

Embodiment I-17. The working electrode of embodiment I-1, wherein the sensor further comprises a microelectrode array, and wherein the working electrode is part of the microelectrode array.

Embodiment I-18. An analyte sensor configured to generate a signal indicative of a concentration of an analyte in a fluid, the analyte sensor comprising the working electrode of embodiment I-1.

Embodiment I-19. The sensor of embodiment I-18, further comprising a microneedle, wherein the working electrode is coupled to the microneedle.

Embodiment I-20. The sensor of embodiment I-19 further comprising a reference electrode and a counter electrode.

Embodiment I-21. A method of manufacturing a working electrode for an analyte sensor configured to generate a signal indicative of a concentration of an analyte in a fluid, the method comprising:
providing a working electrode comprising an electrode material and a biorecognition element deposited on the electrode material, wherein the biorecognition element is configured to selectively and reversibly bind to the analyte;
applying, for a first predetermined time period, a first thiol-based passivation molecule to the electrode material; and
applying, for a second predetermined time period, a second, different thiol-based passivation molecule to the electrode material.

Embodiment I-22. The method of embodiment I-21, wherein applying comprises spray coating one or more of the first thiol-based passivation molecule and the second, different thiol-based passivation molecule on the electrode material.

Embodiment I-23. The method of embodiment I-21, wherein applying comprises submerging the electrode material in a solution comprising one or more of the first thiol-based passivation molecule and the second, different thiol-based passivation molecule.

Embodiment I-24. The method of embodiment I-21, wherein the first thiol-based passivation molecule and the second, different thiol-based passivation molecule are each selected from the group consisting of: 1-hexanethiol, 6-mercapto-1-hexanamine, 6-mercapto-1-phosphatidylcholine hexane, 6-mercapto-1-hexanol (MCH), 7-mercapto-1-heptanol, 8-mercapto-1-octanol (MCO), 9-mercapto-1-nonanol, 10-mercapto-1-decanol, 11-mercapto-1-undecanol, 6-amino-1-hexanethiol, 7-amino-1-heptanethiol, 8-amino-1-octanethiol, 9-amino-1-nonanethiol, 10-amino-1-decanethiol, 11-amino-1-undecanethiol, (6-mercaptohexyl)-N,N,N-trimethylammonium bromide, (7-mercaptoheptyl)-N,N,N-trimethylammonium bromide, (8-mercaptooctyl)-N,N,N-trimethylammonium bromide, (9-mercaptononyl)-N,N,N-trimethylammonium bromide, (10-mercaptodecyl)-N,N,N-trimethylammonium bromide, (11-mercaptoundecyl)-N,N,N-trimethylammonium bromide, 6-mercaptohexylphosphoric acid, 7-mercaptoheptylphosphoric acid, 8-mercaptooctylphosphoric acid, 9-mercaptononylphosphoric acid, 10-mercaptodecylphosphoric acid, 11-mercaptoundecylphosphoric acid, 6-mercaptohexanoic acid, 7-mercaptoheptanoic acid, 8-mercaptooctanoic acid, 9-mercaptononanoic acid, 10-mercaptodecanoic acid, 11-mercaptoundecanoic acid, 6-mercaptohexanesulfonate, 7-mercaptoheptanesulfonate, 8-mercaptooctanesulfonate, 9-mercaptononanesulfonate, 10-mercaptodecanesulfonate, 11-mercaptoundecanesulfonate, poly(ethylene glycol) dithiol, 1,6-hexanedithiol, 1,7-heptanedithiol, 1,8-octanedithiol, 1,9-nonanedithiol, 1,10-decanedithiol, 1,11-undecanedithiol, 2-((3-((6-mercaptohexyl)thio)-2-methylpropanoyl)oxy)ethyl (2-trimethylammonium)ethyl)phosphate (PC), thiolated oligoethylene glycol (OEG), thiolated polyethylene glycol, (3-((6-mercaptohexyl)thio)-2-methylpropanoate-(2-ethyltrimethylammonium) chloride (AC), (3-((6-mercaptohexyl)thio)-2-methylpropanoate-(2-ethylsulfonate) potassium (SP), and (3-((6-mercaptohexyl)thio)-2-methylpropanoate-(2-ethyldimethylammonio-(3-propanesulfonate)) (AP), 1,2-bis-(11-sulfanyloundecanoyl)-sn-glycero-3-phosphocholine, methacrylate alkyl thiol ester, methacrylate PEG thiol ester, acrylate alkyl thiol ester, acrylate PEG thiol ester, vinyl terminated alkyl thiol, vinyl terminated PEG thiol, acetylene terminated alkyl thiol, acetylene terminated PEG thiol, benzophenone terminated alkyl thiol, benzophenone terminated PEG thiol, azide terminated alkyl thiol, azide terminated PEG thiol, N-hydroxysuccinimide terminated alkyl thiol, N-hydroxysuccinimide terminated PEG thiol, ferrocene terminated alkyl thiol, ferrocene terminated PEG thiol, methylene blue terminated alkyl thiol, methylene blue terminated PEG thiol, anthraquinone terminated alkyl thiol, anthraquinone terminated PEG thiol, hydroquinone terminated alkyl thiol, hydroquinone terminated PEG thiol, RGD peptide terminated thiol, and YIGSR peptide terminated thiol.

Embodiment I-25. The method of embodiment I-24, wherein the first thiol-based passivation molecule is MCH and the second, different thiol-based passivation molecule is PC.

Embodiment I-26. The method of embodiment I-25, wherein applying the first thiol-based passivation molecule and the second thiol-based passivation molecule comprises contacting the electrode material with a solution comprising the first thiol-based passivation molecule and the second, different thiol-based passivation molecule at a ratio of MCH to PC of between about 1 mM and about 30 mM MCH to between about 2.5 mM and about 30 mM PC.

Embodiment I-27. The method of embodiment I-25, wherein applying the first thiol-based passivation molecule and the second thiol-based passivation molecule comprises contacting the electrode material with a solution comprising the first thiol-based passivation molecule and the second, different thiol-based passivation molecule at a ratio of MCH to PC of about 15 mM MCH to about 10 mM PC.

Embodiment I-28. The method of embodiment I-21, wherein at least one of the first predetermined time period and the second predetermined time period is between about 4 hours and about 48 hours, between about 12 hours and about 24 hours, or between about 16 hours and about 20 hours.

Embodiment I-29. The method of embodiment I-21, wherein the first thiol-based passivation molecule and the second, different thiol-based passivation molecule are applied to the electrode material until a predetermined density of the passivation layer.

Embodiment I-30. The method of embodiment I-21, wherein applying the first thiol-based passivation molecule and applying the second, different thiol-based passivation molecule are performed at a temperature between about −20° C. and about 37° C., between about 1° C. and about 30° C., or between about 3° C. and about 23° C.

Embodiment I-31. The method of embodiment I-21, wherein the biorecognition element is functionalized with a redox-active molecule and is configured to change conformation upon binding the analyte to move the redox-active molecule closer to or further from the electrode material.

Embodiment I-32. The method of embodiment I-31, wherein the biorecognition element is an aptamer.

Embodiment I-33. The method of embodiment I-31, wherein the redox-active molecule comprises one selected from the group consisting of: methylene blue, ferrocene, pentamethyl ferrocene, C5-ferrocene, Nile blue, thionine, anthraquinone, hydroquinone, C5-anthraquinone, gallocyanine, indophenol, neutral red, dabcyl, carboxy-X-rhodamine, and π extended tetrathiafulvalene (exTTF).

Embodiment I-34. The method of embodiment I-21 further comprising depositing, by chemisorption, a cysteine residue onto the electrode material.

Embodiment I-35. The method of embodiment I-21 further comprising dissolving the first thiol-based passivation molecule in a first solvent; and dissolving the second, different thiol-based passivation molecule in a second solvent.

Embodiment I-36. The method of embodiment I-35, wherein the first solvent and the second solvent are each independently an organic solvent or an inorganic solvent.

Embodiment I-37. The method of embodiment I-35, wherein the first solvent and the second solvent are each independently selected from the group consisting of: ethanol, methanol, propanol, isopropanol, butanol, acetone, acetonitrile, dimethylsulfoxide, dimethylformamide, dimethylacetamide, propylene carbonate, ethylene carbonate, deionized water, phosphate buffered saline, and a buffered salt solution.

Embodiment I-38. The method of embodiment I-35, wherein at least one of the first solvent and the second solvent comprise an organic solvent in water at a concentration of at least about 5% v/v, at least about 10% v/v, at least about 15% v/v, at least about 20% v/v, at least about 25% v/v, at least about 30% v/v, at least about 35% v/v, at least about 40% v/v, at least about 45% v/v, at least about 50% v/v, at least about 55% v/v, at least about 60% v/v, at least about 65% v/v, at least about 70% v/v, at least about 75% v/v, at least about 80% v/v, at least about 85% v/v, at least about 90% v/v, or at least about 95% v/v.

Embodiment I-39. The method of embodiment I-35, wherein the first solvent and the second solvent are the same.

Embodiment I-40. The method of embodiment I-21, wherein the first thiol-based passivation molecule and the second, different thiol-based passivation molecule are applied concurrently.

Embodiment I-41. The method of embodiment I-40, wherein the first thiol-based passivation molecule and the second, different thiol-based passivation molecule are concurrently applied within the same solution.

Embodiment I-42. The method of embodiment I-21, wherein applying the first thiol-based passivation molecule and the second thiol-based passivation molecule comprises contacting the electrode material with a solution comprising the first thiol-based passivation molecule and the second, different thiol-based passivation molecule.

Embodiment I-43. The method of embodiment I-42, wherein at least one of the first thiol-based passivation molecule and the second thiol-based passivation molecule within the solution have enhanced solubility.

Embodiment I-44. A wearable device comprising:
an electrochemical, aptamer-based sensor comprising a first thiol-based passivation molecule and a second, different thiol-based passivation molecule on the electrode material, the first thiol-based passivation molecule comprising 6-mercapto-1-hexanol (MCH) and the second, different thiol-based passivation molecule comprising 2-((3-((6-mercaptohexyl)thio)-2-methylpropanoyl)oxy)ethyl (2-trimethylammonium)ethyl)phosphate (PC).

Embodiment I-45. The wearable device of embodiment I-44, wherein the MCH and the PC are applied to the electrode material at a ratio of MCH to PC of between about 1 mM and about 30 mM MCH to between about 2.5 mM and about 30 mM PC.

Embodiment I-46. The wearable device of embodiment I-44, wherein the MCH and the PC are applied to the electrode material at a ratio of MCH to PC of 15 mM MCH to 10 mM PC.

Embodiment I-47. The wearable device of embodiment I-44, wherein the first thiol-based passivation molecule and the second, different thiol-based passivation molecule on the electrode material maintain stability of the sensor for greater than 150 hours, wherein stability is determined by a comparison of an oxygen reduction reaction-based electrical current and a predefined threshold.

Embodiment I-48. A sensor configured to generate a signal that is indicative of a concentration of an analyte in a fluid, the sensor comprising a working electrode comprising:
an electrode material;
a biorecognition layer disposed at least partially on the electrode material and comprising a biorecognition element that selectively and reversibly binds to an analyte; and
an at least partially desiccated hydrogel disposed on the biorecognition layer.

Embodiment I-49. The sensor of embodiment I-48, wherein the hydrogel comprises about 10%, about 20%, about 30%, about 40%, about 50%, about 60%, about 70%, about 80%, or about 90% of water compared to a fully hydrated state of the hydrogel.

Embodiment I-50. The sensor of embodiment I-48, wherein the hydrogel is fully desiccated.

Embodiment I-51. The sensor of embodiment I-48, wherein the hydrogel comprises one or a combination of two or more hydrophilic polymers selected from the group consisting of: agarose, poly(urethane), poly(N-vinylpyrrolidone), poly(acrylamide), poly(acrylic acid), poly(methacrylic acid), poly(2-hydroxyethyl methacrylate), poly(acrylic acid-co-acrylamide), poly(N-isopropyl acrylamide), poly(2-acrylamido-2-methylpropane sulfonic acid), poly(ethylene glycol), poly(vinyl alcohol), poly(lactic acid), poly(glycolic acid), poly(glycolic acid-co-lactic acid), collagen, alginate, hyaluronic acid, heparin, glycosaminoglycans, chitosan, Nafion, carboxymethylcellulose, and cellulose acetate.

Embodiment I-52. The sensor of embodiment I-48, wherein the hydrogel has a thickness of between about 0.001 μm and about 1000 μm, between about 0.01 μm and about 100 μm, between about 0.1 μm and about 10 μm, between about 0.05 μm and about 500 μm, between about 0.1 μm and about 200 μm, or between about 1 μm and about 1000 μm.

Embodiment I-53. The sensor of embodiment I-48, wherein the working electrode is configured as a planar microelectrode.

Embodiment I-54. The sensor of embodiment I-53, wherein the sensor further comprises a microelectrode array, and wherein the working electrode is part of the microelectrode array.

Embodiment I-55. The sensor of embodiment I-48, wherein the sensor further comprises a microneedle, and wherein the working electrode is coupled to the microneedle.

Embodiment I-56. The sensor of embodiment I-55, wherein the sensor further comprises a microneedle array, and wherein the microneedle is part of the microneedle array.

Embodiment I-57. The sensor of embodiment I-48, wherein the biorecognition element is an aptamer.

Embodiment I-58. A method of manufacturing a sensor configured to generate a signal that is indicative of a concentration of an analyte in a fluid, the method comprising:
(a) providing a working electrode comprising an electrode material and a biorecognition layer disposed at least partially on the electrode material, wherein the biorecognition layer comprises a biorecognition element that selectively and reversibly binds to an analyte;
(b) applying a hydrogel on the biorecognition layer; and
(c) drying the hydrogel to an at least partially desiccated state.

Embodiment I-59. The method of embodiment I-58, wherein the biorecognition layer further comprises a passivation element disposed at least partially on the electrode material.

Embodiment I-60. The method of embodiment I-58 wherein, the hydrogel in the at least partially desiccated state comprises about 10%, about 20%, about 30%, about 40%, about 50%, about 60%, about 70%, about 80%, or about 90% of water compared to a fully hydrated state of the hydrogel.

Embodiment I-61. The method of embodiment I-58, wherein the hydrogel is fully desiccated.

Embodiment I-62. The method of embodiment I-58, wherein the hydrogel comprises one or a combination of two or more of hydrophilic polymers selected from the group consisting of: an agarose, a poly(urethane), a poly(N-vinylpyrrolidone), a poly(acrylamide), a poly(acrylic acid), a poly(methacrylic acid), a poly(2-hydroxyethyl methacrylate), a poly(acrylic acid-co-acrylamide), a poly(N-isopropyl acrylamide), a poly(2-acrylamido-2-methylpropane sulfonic acid), a poly(ethylene glycol), a poly(vinyl alcohol), a poly(lactic acid), a poly(glycolic acid), a poly(glycolic acid-co-lactic acid), a collagen, an alginate, a hyaluronic acid, a heparin, a glycosaminoglycan, a chitosan, Nafion, a carboxymethylcellulose, and a cellulose acetate.

Embodiment I-63. The method of embodiment I-58, wherein the working electrode is configured as a planar microelectrode.

Embodiment I-64. The method of embodiment I-63, wherein the sensor further comprises a microelectrode array, and wherein the working electrode is part of the microelectrode array.

Embodiment I-65. The method of embodiment I-58, wherein the working electrode is coupled to the microneedle.

Embodiment I-66. The method of embodiment I-65, wherein the sensor further comprises a microneedle array, and wherein the microneedle is part of the microneedle array.

Embodiment I-67. The method of embodiment I-58, wherein the hydrogel in the at least partially desiccated state has a thickness of between about 0.001 μm and about 1000 μm, between about 0.01 μm and about 100 μm, between about 0.1 μm and about 10 μm, between about 0.05 μm and about 500 μm, between about 0.1 μm and about 200 μm, or between about 1 μm and about 1000 μm.

Embodiment I-68. The method of embodiment I-58, wherein the hydrogel is applied by drop casting, spin coating, spray coating, chemical vapor deposition (CVD), or dip coating.

Embodiment I-69. The method of embodiment I-58, wherein the hydrogel comprises an agarose.

Embodiment I-70. The method of embodiment I-58, wherein the agarose is a low electroendosmosis agarose characterized by an electroendosmosis of between about 0.09 and about 0.14.

Embodiment I-71. The method of embodiment I-58, wherein, at the time the hydrogel is applied on the biorecognition layer, the hydrogel comprising the agarose at a concentration between about 0.5% w/w and about 4% w/w in a buffered saline solution.

Embodiment I-72. The method of embodiment I-71, wherein the buffered saline is a phosphate buffered saline.

Embodiment I-73. The method of embodiment I-72, wherein the buffered saline is an acetate buffer, a Tris buffer, a citric acid buffer, a Mcllvaine buffer, a Tris-Acetate-EDTA buffer or a Tris-EDTA buffer.

Embodiment I-74. The method of embodiment I-58, wherein the hydrogel is drop casted on the biorecognition layer at a temperature of between about 45 degrees Celsius and about 75 degrees Celsius.

Embodiment I-75. The method of embodiment I-74, wherein the agarose has a melting point of between about 75 degrees Celsius and about 97 degrees Celsius.

Embodiment I-76. The method of embodiment I-75, wherein the hydrogel is applied by dip coating or spin coating.

Embodiment I-77. The method of embodiment I-76, wherein the agarose has a sulfate content of less than about 0.20% w/w.

Embodiment I-78. The method of embodiment I-58, wherein the hydrogel is dried for at least 10 hours at an ambient temperature of between about 15 degrees Celsius and about 30 degrees Celsius, and an ambient humidity of between about 10% and about 80% relative humidity.

Embodiment I-79. The method of embodiment I-78, wherein the ambient humidity is between about 50% and about 80% relative humidity.

Embodiment I-80. The method of embodiment I-79 further comprising packaging the sensor in a sealed disposable package, wherein the interior of the package has an ambient humidity between about 50% and about 80% relative humidity.

Embodiment I-81. The method of embodiment I-58, wherein the biorecognition element is an aptamer.

Embodiment I-82. A sensor comprising:
a working electrode comprising an electrode material, a biorecognition layer disposed at least partially on the electrode material and comprising a biorecognition element that selectively and reversibly binds to an analyte, and a hydrogel disposed on the biorecognition layer,
wherein the sensor is sterilized with exposure to a radiation, and the sterilized sensor is configured to generate a signal that is indicative of a concentration of an analyte in a fluid.

Embodiment I-83. The sensor of embodiment I-82, wherein the biorecognition layer further comprises a passivation element disposed at least partially on the electrode material.

Embodiment I-84. The sensor of embodiment I-82, wherein the radiation is an ultraviolet radiation, a gamma radiation, an X-ray radiation, or an electron beam radiation.

Embodiment I-85. The sensor of embodiment I-84, wherein the radiation is electron beam radiation.

Embodiment I-86. The sensor of embodiment I-85, wherein the electron beam radiation is applied to the working electrode at a dose of between about 2 megarads and about 4 megarads, between about 2.5 megarads and about 3.5 megarads, or between about 2.7 megarads and about 3.1 megarads.

Embodiment I-87. The sensor of embodiment I-82, wherein the hydrogel comprises one or a combination of two or more of hydrophilic polymers selected from the group consisting of: an agarose, a poly(urethane), a poly(N-vinylpyrrolidone), a poly(acrylamide), a poly(acrylic acid), a poly(methacrylic acid), a poly(2-hydroxyethyl methacrylate), a poly(acrylic acid-co-acrylamide), a poly(N-isopropyl acrylamide), a poly(2-acrylamido-2-methylpropane sulfonic acid), a poly(ethylene glycol), a poly(vinyl alcohol), a poly(lactic acid), a poly(glycolic acid), a poly(glycolic acid-co-lactic acid), a collagen, an alginate, a hyaluronic acid, a heparin, a glycosaminoglycan, a chitosan, Nafion, a carboxymethylcellulose, and a cellulose acetate.

Embodiment I-88. The sensor of embodiment I-82, wherein the working electrode is a planar microelectrode.

Embodiment I-89. The sensor of embodiment I-88, wherein the sensor further comprises a microelectrode array, and wherein the working electrode is part of the microelectrode array.

Embodiment I-90. The sensor of embodiment I-82, wherein the sensor comprises a microneedle, and the working electrode is coupled to the microneedle.

Embodiment I-91. The sensor of embodiment I-90, wherein the sensor comprises a microneedle array, and the microneedle is part of the microneedle array.

Embodiment I-92. The sensor of embodiment I-82, wherein the hydrogel has a thickness of between about 0.001 μm and about 1000 μm, between about 0.01 μm and about 100 μm, between about 0.1 μm and about 10 μm, between about 0.05 μm and about 500 μm, between about 0.1 μm and about 200 μm, or between about 1 μm and about 1000 μm.

Embodiment I-93. The sensor of embodiment I-82, wherein the biorecognition layer is degraded by the radiation exposure by not more than about 2%, not more than about 5%, not more than about 10%, not more than about 15%, or not more than about 20%.

Embodiment I-94. The sensor of embodiment I-93, wherein degradation of the biorecognition layer is determined by:
placing the working electrode in a fluid comprising an analyte and gathering a signal from the working electrode indicative of a concentration of the analyte in the fluid; and
comparing the strength of the signal to a reference signal strength.

Embodiment I-95. The sensor of embodiment I-94, wherein the reference signal strength is based on an initial signal measured by the same or equivalent working electrode placed in an equivalent fluid, wherein the same or equivalent working electrode has not been sterilized with exposure to a radiation.

Embodiment I-96. The method of embodiment I-82, wherein the biorecognition element is an aptamer.

Embodiment I-97. A method of sterilizing a sensor configured to generate a signal that is indicative of a concentration of an analyte in a fluid, the method comprising:
(a) providing a working electrode comprising an electrode material and a biorecognition layer comprising a biorecognition element that selectively and reversibly binds to an analyte, wherein the biorecognition layer is disposed at least partially on the electrode material;
(b) applying a hydrogel on the biorecognition layer; and
(c) sterilizing the working electrode with exposure to a radiation.

Embodiment I-98. The method of embodiment I-97, wherein the biorecognition layer further comprises a passivation element disposed at least partially on the electrode material.

Embodiment I-99. The method of embodiment I-97, wherein the radiation is ultraviolet radiation, gamma radiation, X-ray radiation, or electron beam radiation.

Embodiment I-100. The method of embodiment I-99, wherein the radiation is electron beam radiation.

Embodiment I-101. The method of embodiment I-100, wherein the electron beam radiation is applied to the working electrode at a dose of between about 2 megarads and about 4 megarads, between about 2.5 megarads and about 3.5 megarads, or between about 2.7 megarads and about 3.1 megarads.

Embodiment I-102. The method of embodiment I-97, wherein the hydrogel comprises one or a combination of two or more of hydrophilic polymers selected from the group consisting of: an agarose, a poly(urethane), a poly(N-vinylpyrrolidone), a poly(acrylamide), a poly(acrylic acid), a poly(methacrylic acid), a poly(2-hydroxyethyl methacrylate), a poly(acrylic acid-co-acrylamide), a poly(N-isopropyl acrylamide), a poly(2-acrylamido-2-methylpropane sulfonic acid), a poly(ethylene glycol), a poly(vinyl alcohol), a poly(lactic acid), a poly(glycolic acid), a poly(glycolic acid-co-lactic acid), a collagen, an alginate, a hyaluronic acid, a heparin, a glycosaminoglycan, a chitosan, Nafion, a carboxymethylcellulose, and a cellulose acetate.

Embodiment I-103. The method of embodiment I-97, wherein the working electrode is a planar microelectrode.

Embodiment I-104. The method of embodiment I-103, wherein the sensor further comprises a microelectrode array, and wherein the working electrode is part of the microelectrode array.

Embodiment I-105. The method of embodiment I-97, wherein the working electrode is coupled to a microneedle.

Embodiment I-106. The method of embodiment I-105, wherein the microneedle is part of a microneedle array.

Embodiment I-107. The method of embodiment I-97, wherein the hydrogel has a thickness of between about 0.001 μm and about 1000 μm, between about 0.01 μm and about 100 μm, between about 0.1 μm and about 10 μm, between about 0.05 μm and about 500 μm, between about 0.1 μm and about 200 μm, or between about 1 μm and about 1000 μm.

Embodiment I-108. The method of embodiment I-97, wherein the biorecognition layer is degraded by the radiation exposure by not more than about 2%, not more than about 5%, not more than about 10%, not more than about 15%, or not more than about 20%.

Embodiment I-109. The method of embodiment I-108, wherein degradation of the biorecognition layer is determined by:
placing the working electrode in a fluid comprising an analyte and gathering a signal from the working electrode indicative of a concentration of the analyte in the fluid; and
comparing the strength of the signal to a reference signal strength.

Embodiment I-110. The method of embodiment I-109, wherein the reference signal strength is based on an initial signal measured by the same or equivalent working electrode placed in an equivalent fluid, wherein the same or equivalent working electrode has not been sterilized with exposure to a radiation.

Embodiment I-111. The method of embodiment I-97, wherein the biorecognition element is an aptamer.

Embodiment I-112. A sensor configured to generate a signal that is indicative of a concentration of an analyte in a fluid, the sensor comprising a working electrode comprising:
an electrode material;
a biorecognition layer disposed at least partially on the electrode material and comprising a biorecognition element that selectively and reversibly binds to an analyte; and
a hydrogel disposed on the biorecognition layer, wherein
the biorecognition layer degrades at a rate of not more than about 3% per day when stored at an ambient temperature of between about 15 degrees Celsius and about 30 degrees Celsius, and an ambient humidity of between about 10% and about 80% relative humidity.

Embodiment I-113. The sensor of embodiment I-112, wherein the biorecognition layer further comprises a passivation element disposed at least partially on the electrode material.

Embodiment I-114. The sensor of embodiment I-112, wherein the hydrogel is in an at least partially desiccated state.

Embodiment I-115. The sensor of embodiment I-114, wherein the hydrogel in the at least partially desiccated state comprises about 10%, about 20%, about 30%, about 40%, about 50%, about 60%, about 70%, about 80%, or about 90% of water compared to a fully hydrated state of the hydrogel.

Embodiment I-116. The sensor of embodiment I-114, wherein the hydrogel is in a fully desiccated state.

Embodiment I-117. The sensor of embodiment I-115, wherein degradation of the biorecognition layer is measured based on a reduction of a signal produced by the working electrode when placed in a liquid comprising the analyte compared to a reference signal strength.

Embodiment I-118. The sensor of embodiment I-117, wherein the reference signal strength is based on:
an initial signal measured by the same or an equivalent working electrode placed in an equivalent fluid within 4 hours of the hydrogel being applied on the biorecognition layer; or
a comparative signal measured by an equivalent working electrode stored for an equivalent time period and placed in an equivalent fluid, wherein the equivalent sensor lacks a hydrogel disposed on the biorecognition layer.

Embodiment I-119. The sensor of embodiment I-112, wherein the hydrogel comprises one or a combination of two or more of hydrophilic polymers selected from the group consisting of: agarose, poly(urethane), poly(N-vinylpyrrolidone), poly(acrylamide), poly(acrylic acid), poly(methacrylic acid), poly(2-hydroxyethyl methacrylate), poly(acrylic acid-co-acrylamide), poly(N-isopropyl acrylamide), poly(2-acrylamido-2-methylpropane sulfonic acid), poly(ethylene glycol), poly(vinyl alcohol), poly(lactic acid), poly(glycolic acid), poly(glycolic acid-co-lactic acid), collagen, alginate, hyaluronic acid, heparin, glycosaminoglycans, chitosan, Nafion, carboxymethylcellulose, and cellulose acetate.

Embodiment I-120. The sensor of embodiment I-114, wherein the hydrogel in the at least partially desiccated state has a thickness of between about 0.01 μm and about 100 μm, between about 0.1 μm and about 10 μm, between about 0.05 μm and about 500 μm, between about 0.1 μm and about 200 μm, or between about 1 μm and about 1000 μm.

Embodiment I-121. The sensor of embodiment I-118, wherein the working electrode is a planar microelectrode.

Embodiment I-122. The sensor of embodiment I-121, wherein the sensor further comprises a microelectrode array, and wherein the working electrode is part of the microelectrode array.

Embodiment I-123. The sensor of embodiment I-112, wherein the sensor further comprises a microneedle, and wherein the working electrode is coupled to the microneedle.

Embodiment I-124. The sensor of embodiment I-123, wherein the sensor further comprises a microneedle array, and wherein the microneedle is part of the microneedle array.

Embodiment I-125. The sensor of embodiment I-112, wherein the biorecognition element is an aptamer.

Embodiment I-126. A working electrode configured to generate a signal indicative of a concentration of an analyte in a fluid, the working electrode comprising:
an electrode material;
a biorecognition element disposed on the electrode material that selectively binds to the analyte, wherein the biorecognition element is functionalized with a redox-active molecule and is configured to change conformation upon binding the analyte to move the redox-active molecule, closer to, or further from, the electrode material; and
a passivation element comprising a zwitterionic peptide disposed on the electrode material.

Embodiment I-127. The working electrode of embodiment I-126, wherein the zwitterionic peptide comprises at least one amino acid with a carboxyl group as a side chain.

Embodiment I-128. The working electrode of embodiment I-127, wherein at least one amino acid with a carboxyl group as a side chain comprises a glutamate residue, an aspartate residue, or a combination thereof.

Embodiment I-129. The working electrode of any one of embodiments I-126 to I-128, wherein the zwitterionic peptide comprises at least one amino acid with a positively charged side chain.

Embodiment I-130. The working electrode of any one of embodiments I-126 to I-129, wherein the positively charged side chain is an amine group, a guanidino group, or an imidazole group.

Embodiment I-131. The working electrode of embodiment I-129 or I-130, wherein the at least one amino acid with a positively charged side chain is one of or a combination of two or more of a lysine residue, a histidine residue, an ornithine residue, and an arginine residue.

Embodiment I-132. The working electrode of any one of embodiments I-126 to I-131, wherein the zwitterionic peptide comprises a thiol group.

Embodiment I-133. The working electrode of embodiment I-132, wherein the zwitterionic peptide comprises a cysteine residue, and the thiol group is a side chain of the cysteine residue.

Embodiment I-134. The working electrode of embodiment I-133, wherein the thiol group comprises an extended carbon chain.

Embodiment I-135. The working electrode of embodiment I-134, wherein the extended carbon chain is a chain of between one and 20 carbon atoms.

Embodiment I-136. The working electrode of embodiment I-133, wherein the cysteine residue is a C-terminal cysteine residue or an N-terminal cysteine.

Embodiment I-137. The working electrode of embodiment I-136, wherein the C-terminus of the C-terminal cysteine is a free carboxyl group.

Embodiment I-138. The working electrode of embodiment I-137, wherein the C-terminus of the C-terminal cysteine a modified C-terminus having a neutral charge.

Embodiment I-139. The working electrode of embodiment I-138, wherein the modified C-terminus having the neutral charge is an amide group, an ester, a methoxy ester, or a methoxide group.

Embodiment I-140. The working electrode of embodiment I-136, wherein the N-terminus of the N-terminal cysteine is a free amine group.

Embodiment I-141. The working electrode of embodiment I-140, wherein the N-terminus of the N-terminal cysteine is a modified N-terminus having a neutral charge.

Embodiment I-142. The working electrode of embodiment I-141, wherein the modified N-terminus having the neutral charge is an amide group or an alkylamine group.

Embodiment I-143. The working electrode of embodiment I-142, wherein the amide group is an acetamide group.

Embodiment I-144. The working electrode of any one of embodiments I-126 to I-143, wherein the zwitterionic peptide comprises at least one non-canonical amino acid.

Embodiment I-145. The working electrode of embodiment I-144, wherein the at least one non-canonical amino acid is selection from: ornithine, Beta-alanine, γ-aminobutyric acid (GABA), 4-aminobenzoic acid, taurine, or a combination thereof.

Embodiment I-146. The working electrode of any one of embodiments I-126 to I-143, wherein the zwitterionic peptide consists of a peptide sequence of X-(KX)ₓ-P_{y}C or CP_{y}-(KX)ₓ-K, wherein x is between 1 and 5, X is a glutamic acid residue or an aspartic acid residue, K is a lysine residue, P is a proline residue, and C is a cysteine residue.

Embodiment I-147. The working electrode of embodiment I-146, wherein y is 5 or less.

Embodiment I-148. The working electrode of embodiment I-146, wherein y is 3 or less.

Embodiment I-149. The working electrode of any one of embodiments I-133 to I-148, wherein the cysteine residue is chemisorbed to the electrode material.

Embodiment I-150. The working electrode of any one of embodiments I-126 to I-149, wherein the biorecognition element is an aptamer.

Embodiment I-151. The working electrode of embodiment I-150, wherein the aptamer comprises a thiol group at 3′ or 5′ terminal and the thiol group is chemisorbed to the electrode material.

Embodiment I-152. The working electrode of any one of embodiments I-126 to I-151, wherein the electrode material is gold or glassy carbon.

Embodiment I-153. The working electrode of any one of embodiments I-126 to I-152, wherein the redox-active molecule is selected from the group consisting of: methylene blue, ferrocene, pentamethyl ferrocene, C5-ferrocene, Nile blue, thionine, anthraquinone, C5-anthraquinone, gallocyanine, indophenol, neutral red, dabcyl, carboxy-X-rhodamine, and π extended tetrathiafulvalene (exTTF).

Embodiment I-154. The working electrode of any one of embodiments I-126 to I-153, wherein the zwitterionic peptide is deposited on a surface of the electrode material at a concentration of between about 1 mM and about 30 mM.

Embodiment I-155. An analyte sensor comprising the working electrode of any one of embodiments I-126 to I-154, a counter electrode, and a reference electrode.

Embodiment I-156. The analyte sensor of embodiment I-155, wherein the electrode material is coupled to a microneedle.

Embodiment I-157. The analyte sensor of embodiment I-156, wherein the analyte sensor further comprises a microneedle array, wherein the microneedle is a first microneedle of the microneedle array.

Embodiment I-158. The analyte sensor of embodiment I-157, wherein the counter electrode is coupled to a second microneedle of the microneedle array and the reference electrode is coupled to a third microneedle of the microneedle array.

Embodiment I-159. The analyte sensor of any one of embodiments I-126 to I-158, wherein the first microneedle comprises a tapered distal portion having an insulated distal apex and the working electrode is located on a surface of the tapered distal portion that is proximal to the insulated distal apex.

Embodiment I-160. The analyte sensor of embodiment I-159, wherein the working electrode is an annular electrode comprising a proximal edge and a distal edge, and the distal edge of the annular working electrode is proximate a proximal edge of the insulated distal apex.

Embodiment I-161. A working electrode configured to generate a signal indicative of a concentration of an analyte in a fluid, the working electrode comprising:
an electrode material;
a biorecognition element disposed on the electrode material that selectively binds to the analyte; and
a passivation element comprising a zwitterionic peptide disposed on the electrode material that comprises an amino acid sequence of X-(KX)ₓ-P_{y}C or CP_{y}-(KX)ₓ-K, wherein x is 0, 1 or 2, X is a glutamic acid residue or an aspartic acid residue, K is a lysine residue, P is a proline residue, and C is a cysteine residue.

Embodiment I-162. The working electrode of embodiment I-161, wherein cysteine residue comprises a thiol group comprising an extended carbon chain.

Embodiment I-163. The working electrode of embodiment I-162, wherein the extended carbon chain is a chain of between one and 20 carbon atoms.

Embodiment I-164. The working electrode of any one of embodiments I-161 to I-163 wherein the amino acid sequence of the zwitterionic peptide is X-(KX)ₓ-P_{y}C comprising a C-terminal cysteine, and the C-terminus of the C-terminal cysteine is a free carboxyl group.

Embodiment I-165. The working electrode of any one of embodiments I-161 to I-163, wherein the C-terminus of the C-terminal cysteine is a modified C-terminus having a neutral charge.

Embodiment I-166. The working electrode of embodiment I-165, wherein the modified C-terminus having the neutral charge is an amide group, an ester, a methoxy ester, or a methoxide group.

Embodiment I-167. The working electrode of any one of embodiments I-161 to I-163, wherein the amino acid sequence of the zwitterionic peptide is CP_{y}-(KX)ₓ-K comprising an N-terminal cysteine, and the N-terminus of the N-terminal cysteine residue is a free amine group.

Embodiment I-168. The working electrode of any one of embodiments I-161 to I-163, wherein the amino acid sequence of the zwitterionic peptide is CP_{y}-(KX)ₓ-K comprising an N-terminal cysteine, and the N-terminus of the N-terminal cysteine is a modified N-terminus having a neutral charge.

Embodiment I-169. The working electrode of embodiment I-168, wherein the modified N-terminus having the neutral charge is an amide group or an alkylamine group.

Embodiment I-170. The working electrode of embodiment I-169, wherein the amide group is an acetamide group.

Embodiment I-171. The working electrode of any one of embodiments I-161 to I-170, wherein y is 3 or less.

Embodiment I-172. The working electrode of embodiment I-171, wherein the zwitterionic peptide consists of the amino acid sequence of XKXKXPPC.

Embodiment I-173. The working electrode of embodiment I-171, wherein the zwitterionic peptide consists of the amino acid sequence of XKXPPC.

Embodiment I-174. The working electrode of embodiment I-161, wherein the zwitterion peptide consists of the amino acid sequence of any one of SEQ ID NO: 2-33.

Embodiment I-175. The working electrode of any one of embodiments I-168 to I-174, wherein the cysteine residue is chemisorbed to the electrode material.

Embodiment I-176. The working electrode of any one of embodiments I-168 to I-175, wherein the electrode material is gold or glassy carbon.

Embodiment I-177. The working electrode of any one of embodiments I-168 to I-176, wherein the biorecognition element is an aptamer.

Embodiment I-178. The working electrode of embodiment I-177, wherein the aptamer comprises a thiol group at 3′ or 5′ terminal and the thiol group is chemisorbed to the electrode material.

Embodiment I-179. The working electrode of embodiment I-178, wherein the biorecognition layer is substantially free of 6-mercaptohexanol.

Embodiment I-180. The working electrode of any one of embodiments I-161 to I-179, wherein the biorecognition element is functionalized with a redox-active molecule and is configured to experience a conformational change upon binding the analyte such that the redox-active molecule moves closer to, or further from, the electrode material.

Embodiment I-181. The working electrode of embodiment I-180, wherein the redox-active molecule is selected from the group consisting of: methylene blue, ferrocene, pentamethyl ferrocene, C5-ferrocene, Nile blue, thionine, anthraquinone, C5-anthraquinone, gallocyanine, indophenol, neutral red, dabcyl, carboxy-X-rhodamine, and π extended tetrathiafulvalene (exTTF).

Embodiment I-182. The working electrode of any one of embodiments I-161 to I-181, wherein the zwitterionic peptide is deposited on a surface of the electrode material at concentration of between about 1 mM and about 30 mM.

Embodiment I-183. An analyte sensor comprising the working electrode of any one of embodiments I-161 to I-182, a counter electrode, and a reference electrode.

Embodiment I-184. The analyte sensor of embodiment I-183, further comprising a microneedle, wherein the working electrode is coupled to the microneedle.

Embodiment I-185. The analyte sensor of embodiment I-183, wherein the sensor further comprises a microneedle array, wherein the microneedle is a first microneedle of the microneedle array.

Embodiment I-186. The analyte sensor of embodiment I-185, wherein the counter electrode is coupled to a second microneedle of the microneedle array and the reference electrode is coupled to a third microneedle of the microneedle array.

Embodiment I-187. The analyte sensor of any one of embodiments I-183 to I-186, wherein the first microneedle comprises a tapered distal portion having an insulated distal apex, and wherein the working electrode is located on a surface of the tapered distal portion that is proximal to the insulated distal apex.

Embodiment I-188. The analyte sensor of embodiment I-187, wherein the working electrode is an annular electrode comprising a proximal edge and a distal edge, wherein the distal edge of the annular working electrode is proximate a proximal edge of the insulated distal apex.

Embodiment I-189. A zwitterionic peptide comprising an amino acid sequence of X-(KX)ₓ-P_{y}C or CP_{y}-(KX)ₓ-K, wherein x is 0, 1 or 2, X is a glutamic acid residue or an aspartic acid residue, K is a lysine residue, P is a proline residue, and C is a cysteine residue.

Embodiment I-190. The zwitterionic peptide of embodiment I-189, wherein cysteine residue comprises a thiol group comprising an extended carbon chain.

Embodiment I-191. The zwitterionic peptide of embodiment I-190, wherein the extended carbon chain is a chain of between one and 20 carbon atoms.

Embodiment I-192. The zwitterionic peptide of any one of embodiments I-189 to I-191, wherein the amino acid sequence of the zwitterionic peptide is X-(KX)ₓ-P_{y}C comprising a C-terminal cysteine, and the C-terminus of the C-terminal cysteine is a free carboxyl group.

Embodiment I-193. The zwitterionic peptide of any one of embodiments I-189 to I-191, wherein the amino acid sequence of the zwitterionic peptide is X-(KX)ₓ-P_{y}C comprising a C-terminal cysteine, and the C-terminus of the C-terminal cysteine is a modified C-terminus having a neutral charge.

Embodiment I-194. The zwitterionic peptide of embodiment I-193, wherein the modified C-terminus having the neutral charge is an amide group, an ester, a methoxy ester, or a methoxide group.

Embodiment I-195. The zwitterionic peptide of any one of embodiments I-189 to I-191, wherein the amino acid sequence of the zwitterionic peptide is CP_{y}-(KX)ₓ-K comprising an N-terminal cysteine, and the N-terminus of the N-terminal cysteine is a free amine group.

Embodiment I-196. The zwitterionic peptide of any one of embodiments I-190 to I-191, wherein the amino acid sequence of the zwitterionic peptide is CP_{y}-(KX)ₓ-K comprising an N-terminal cysteine, and the N-terminus of the N-terminal cysteine is a modified N-terminus having a neutral charge.

Embodiment I-197. The zwitterionic peptide of embodiment I-196, wherein the modified N-terminus having the neutral charge is an amide group or an alkylamine group.

Embodiment I-198. The zwitterionic peptide of embodiment I-197, wherein the amide group is an acetamide group.

Embodiment I-199. The zwitterionic peptide of any one of embodiment I-189 to I-198, wherein y is 3 or less.

Embodiment I-200. The zwitterionic peptide of embodiment I-199, wherein the zwitterionic peptide consists of the amino acid sequence of XKXKXPPC or XKXPPC.

Embodiment I-201. The zwitterionic peptide of embodiment I-199, wherein the zwitterionic peptide consists of the amino acid sequence of XKXPPC.

Embodiment I-202. The zwitterionic peptide of embodiment I-189, wherein the zwitterion peptide consists of the amino acid sequence of any one of SEQ ID NO: 2-33.

Embodiment I-203. The zwitterionic peptide of any one of embodiments I-189 to I-193 and I-195 to I-202, wherein the cysteine residue is modified with an amide group, an ester, a methoxy ester, or a methoxide group.

Embodiment I-204. A method for manufacturing a working electrode, comprising:
depositing gold on a substrate to create a gold surface;
depositing a biorecognition element on the gold surface, wherein the biorecognition element is functionalized with a redox-active molecule and is configured to change conformation upon binding the analyte to move the redox-active molecule, closer to, or further from, the gold surface; and
depositing a passivation element comprising a zwitterionic peptide on the gold surface.

Embodiment I-205. The method of embodiment I-204, wherein the gold surface is not polished prior to the depositing of the zwitterionic peptide and the biorecognition element.

Embodiment I-206. The method of embodiment I-204 further comprising polishing the gold surface prior depositing the passivation element and the biorecognition element.

Embodiment I-207. The method of embodiment I-206, wherein the polishing comprises electropolishing.

Embodiment I-208. The method of embodiment I-204, wherein the gold is deposited through electrodeposition, chemical vapor deposition, electroless plating, or physical vapor deposition.

### EXAMPLES

### Example 1—Effect of Hydrogel Biocompatible Layer on Sensor Degradation Due to Dry Storage

It was found that a hydrogel biocompatible layer applied on a biorecognition layer of a working electrode comprising an analyte-binding aptamer and configured to be sensitive to analyte concentration did not meaningfully impede the analyte sensitivity of the working electrode. In addition, the presence of the hydrogel biocompatible layer slowed the degradation of the function of the biorecognition layer that typically occurs when the working electrode is stored in air.

As shown by way of example in FIG. 21A, planar gold electrodes 2112 designated to be cortisol-sensitive working electrodes 2110 in a planar gold electrode array were functionalized with an aqueous solution containing an analyte-binding aptamer 2125 with a terminal thiol group (not shown) on one end and a methylene blue 2126 on the other end that served as the redox reporter. Each electrode in the array had a surface area of ˜10,000 μm². The cortisol-binding aptamer 2125 had the following DNA sequence:

(SEQ ID NO: 1)
5′-GGACGACGCCAGAAGTTTACGAGGATATGGTAACATAGTCGT-3′

where G, A, C, and T represent the typical DNA nucleotides containing guanine, adenine, cytosine, and thymine, respectively. The functionalization process included a secondary step (“backfilling”) to functionalize the remainder of the surface of the gold working electrode 2610 with a small molecule thiol, 6-mercapto-1-hexanol as passivation molecules 2129, in order to passivate the surface and/or add biocompatible functionality. Upon binding cortisol 2101, cortisol-binding aptamer 2125 experiences a conformational change that moves the methylene blue closer, or further, from the planar electrode 2112. The methylene blue is held in its oxidized state, and a sweep to more negative potential reduces the redox-active molecule within range of electron transfer. This change in potential can detected as a signal corresponding to the concentration of cortisol at the working electrode 2110 using square wave voltammetry (SWV). The cortisol-binding aptamer 2125 with methylene blue 2626 together with the 6-mercapto-1-hexanol 2629 bound to the planar gold surface 2612 forms the biorecognition layer 2614.

As shown in FIG. 21B, after the formation of the biorecognition layer 2114 through the functionalization of the planar gold electrode 2112 with the cortisol-binding aptamer 2125 and 6-mercapto-1-hexanol 2129, the working electrode 2110 was coated with an agarose hydrogel to form a biocompatible layer 2116. The agarose, initially in powder form, was dissolved at about 90° C. in PBS buffer and drop casted onto to the air-dried electrode array, and allowed to dry overnight exposed to air at 23° C. and 50-55% relative humidity. FIG. 21C shows a schematic illustration of a perspective view of a microelectrode array 2100 used to obtain the results shown below, comprising a plurality of working electrodes 2110 functionalized with the cortisol-binding aptamers (not shown), and a biocompatible layer 2126 covering the working electrodes.

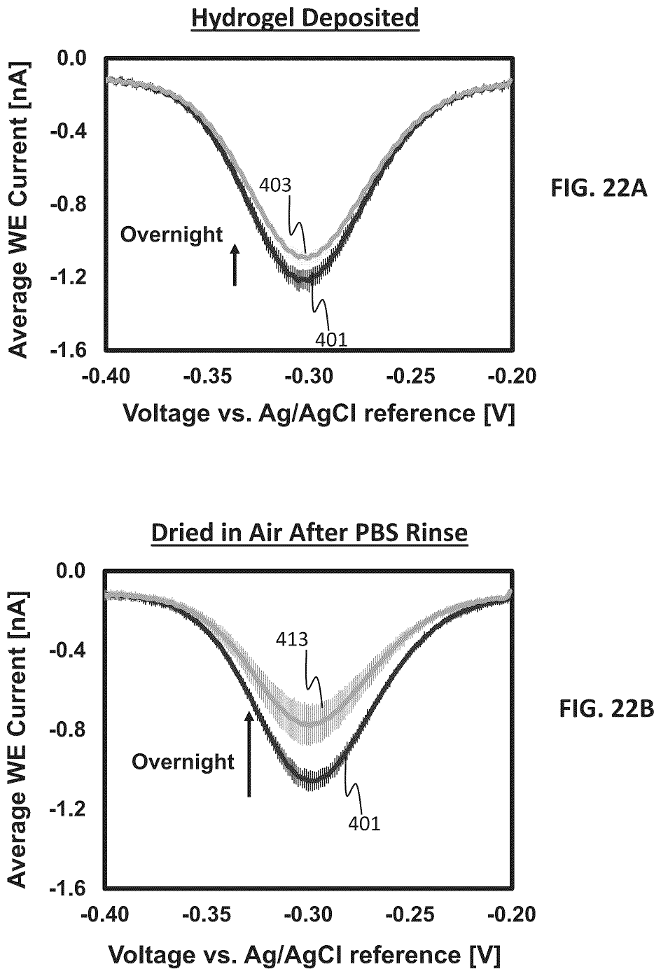

FIGS. 22A-22B show square wave voltammograms showing an average (n=5) SWV current in the presence of 1 μM cortisol measured with working electrode 2610 before and after application of the hydrogel. During the signal measurement, the sensor array was immersed in PBS with 2% dimethyl-sulfoxide (DMSO) with 1 μM cortisol. The square wave frequency used to measure the SWV current was 50 Hz. As can be seen in FIG. 22A, the reduction of the SWV current was not substantial, a signal loss of about 3% when the working electrode was covered with the hydrogel biocompatible layer. Line 401 shows the SWV current generated by the bare working electrode 2110 without hydrogel, and line 403 shows the same signal generated by the hydrogel-covered working electrode after hydrogel application and overnight storage. By contrast, as shown in FIG. 22B, when the air-dried electrode array was left bare and stored overnight without application and drying of the hydrogel biocompatible layer 2116, the peak SWV current in the presence of to 1 μM cortisol was reduced by 25%. Line 411 shows the SWV current in the presence of cortisol generated by the bare working electrode 2110 without hydrogel, and line 413 shows the same signal generated by the bare working electrode after overnight storage.

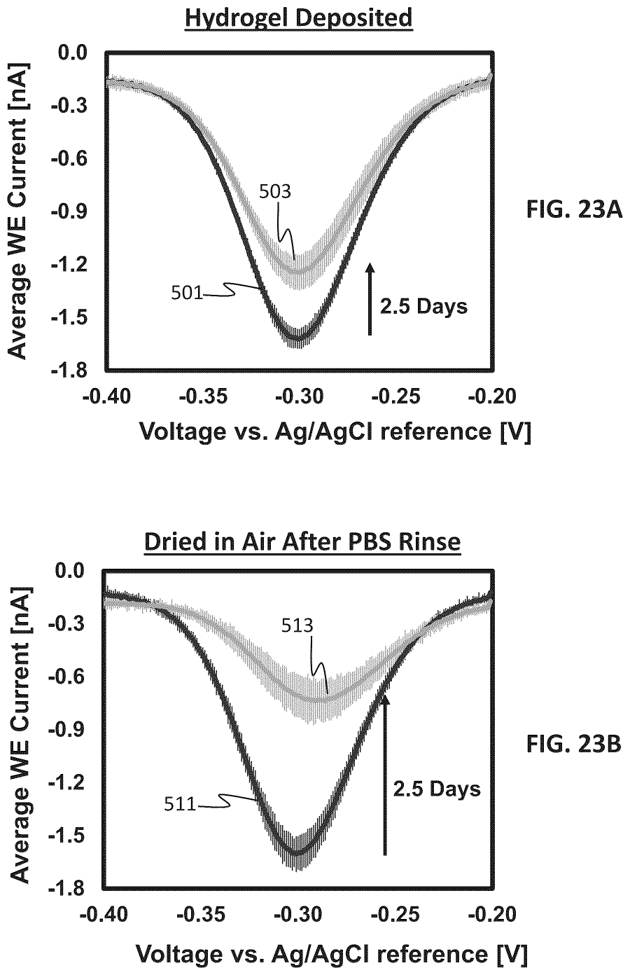

FIGS. 23A-23B show square wave voltammograms demonstrating the degradation of SWV current at in the presence of 1 μM cortisol measured from working electrodes with and without the hydrogel, after dry storage for 2.5 days. During the signal measurement, the sensor array was immersed in PBS with 2% dimethyl-sulfoxide (DMSO) with 1 μM cortisol. The square wave frequency used to measure the peak SWV current was 50 Hz. The peak SWV current from the hydrogel-covered sensor (FIG. 23A) degraded at an average rate (n=5) of 8%/day (compare line 501 showing the SWV current in the presence of cortisol generated by a bare working electrode prior to storage, with line 503 showing the peak SWV current in the presence of cortisol generated by the working electrode after hydrogel application and 2.5 days of dry storage). By contrast, the signal from the bare sensor without hydrogel (FIG. 23B) degraded at an average rate of 25%/day (compare line 511 showing the peak SWV current in the presence of cortisol generated by a bare working electrode prior to storage, with line 513 showing the peak SWV current in the presence of cortisol generated by the bare working electrode without hydrogel application and after 2.5 days of dry storage). Without being bound by a particular theory, it appears the partially desiccated hydrogel retains moisture that assists in stabilizing the molecular components (e.g., the aptamers and/or small molecule thiols) of the biorecognition layer during the dry storage conditions. In addition, the hydrophilic polymer itself may impart some protection to the biorecognition layer.

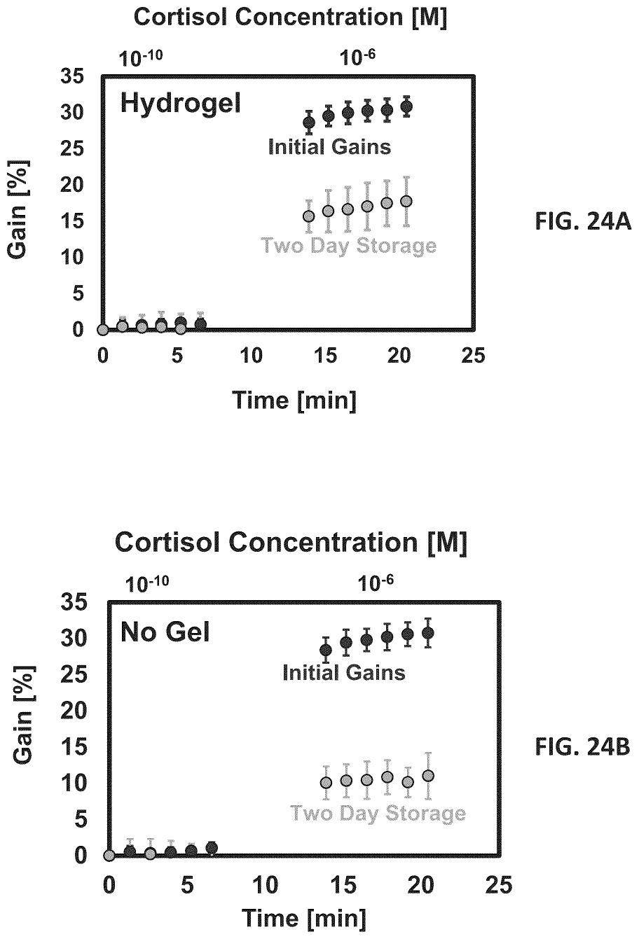

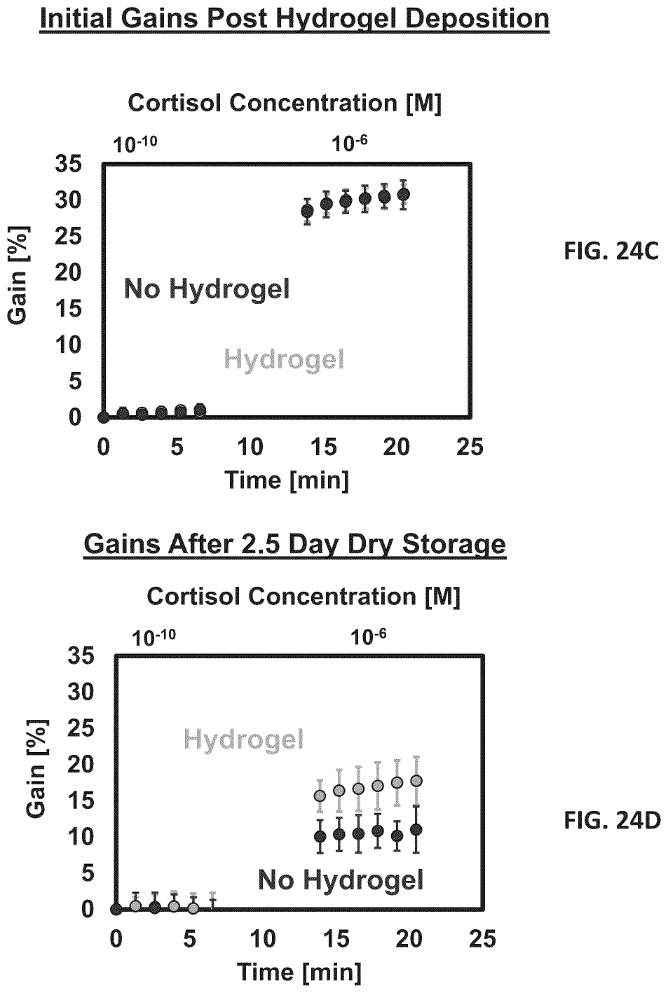

FIGS. 24A-24B shows a difference plot time series showing % changes (“% gain”) in peak SWV current generated in response to cortisol exposure by aptamer-based cortisol sensor arrays in one of four conditions. For the cortisol exposure, sensor arrays were immersed in PBS with 2% DMSO and cortisol at one of two concentrations—a baseline concentration of 100 pM (10⁻¹⁰ M) cortisol followed by A test concentration of 1 μM (10⁻⁶ M) cortisol. For each run, the sensor array was first presented with the baseline cortisol concentration for 7 minutes during which a peak SWV current was measured six times, followed by a change in the immersion medium from the baseline concentration to the test concentration over a second seven-minute session, followed by third seven-minute session when the SWV peak current was measured again for another six times. Error bars represent a single standard deviation across the plurality of working electrodes in the electrode array. The square wave frequency used to measure the peak SWV current was 50 Hz. This peak was background-corrected with a non-cortisol-sensitive background peak that was measured with a secondary square wave frequency of 5 Hz. The % gain at each peak current measurement is the % gain of the peak current measured at the test concentration compared to the baseline concentration, and was calculated as follows, with Aₘₑₐₛᵤᵣₑₘₑₙₜ being the peak SWV current of a given measurement:

$$Gain [ \% ] = \frac{{A}_{measurement} - {A}_{0}}{{A}_{0}} \times 100$$

With Ao being the initial peak SWV currents measure at time=0 minutes, with the sensor array being presented with cortisol at the baseline cortisol concentration. As such, the % gain of each of the baseline measurements centers, as expected, at around 0%. Note that representing the cortisol response as % gain between two cortisol concentrations (e.g. 1 μM cortisol compared to 100 pM cortisol as shown in FIGS. 24A-24B), rather than the raw Aₘₑₐₛᵤᵣₑₘₑₙₜ at a given cortisol concentration, better demonstrates the sensor's ability to detect changes in cortisol concentration given the state of the sensor array.

The four sensor conditions were as follows: (1) newly prepared sensors after 6 hours of functionalization of the working electrodes with cortisol-sensing aptamers, without hydrogel (“bare”); (2) newly prepared sensors with hydrogel application, after approximately 6 hours of hydrogel curing; (3) the bare aptamer-functionalized sensors after 2.5 days of dry storage; and (4) the aptamer-functionalized sensors with hydrogel application after 2.5 days of dry storage.

FIG. 24A overlays the % gain obtained with sensor conditions (1) and (3), comparing the % gain of the cortisol signal between newly prepared sensors without hydrogel and the same sensors after 2.5 days of dry storage. FIG. 24B overlays the % gain obtained with sensor conditions (2) and (4), comparing the % gain of the cortisol signal between newly prepared sensors with the hydrogel coating and the same sensors after 2.5 days of dry storage. FIG. 24A and FIG. 24B show that 2.5 days of dry storage results in sensor performance being reduced in both hydrogel-coated and uncoated sensors.

FIG. 24D overlays the % gain obtained with sensor conditions (3) and (4), comparing the % gain of the cortisol signal between sensors with hydrogel and sensors without hydrogel after 2.5 days of dry storage. FIG. 24B shows that, after the 2.5-day dry storage, the % gain measured with the hydrogel-coated sensor (approximately 17% gain) is more robust than with the sensor without the hydrogel (approximately 11% gain) thus demonstrating the advantage of the hydrogel coating for retaining sensor performance during dry storage.

FIG. 24C overlays the % gain obtained with sensor conditions (1) and (2), comparing the % gain of the cortisol signal between newly prepared sensors with hydrogel and newly prepared sensors without hydrogel. FIG. 24A shows that there is initially no difference in the % gain between the two sensor conditions (the % gain being about 30% in both conditions), thus demonstrating that application of the hydrogel does not impede cortisol diffusion or the cortisol-dependent structural switching of the aptamer, which is key for signal transduction.

### Example 2—Effect of Hydrogel Biocompatible Layer on Sensor Degradation Due to Electron Beam Sterilization

It was found that the presence of a hydrogel biocompatible layer reduced degradation of the biorecognition layer due to radiation exposure. An analyte sensor using the planar gold array with functionalized electrodes with or without the hydrogel biocompatible layer, as described with reference to FIGS. 21A-21C were also used in this example. The sensors were processed in an e-beam sterilization facility. The e-beam exposure parameters were as follows: two passes of the e-beam at an intensity of 12 MeV, with each pass being about 2 seconds. The e-beam exposures were conducted after the sensor arrays were subject to 18 hours of dry storage. The hydrogel layer when present was partially desiccated.

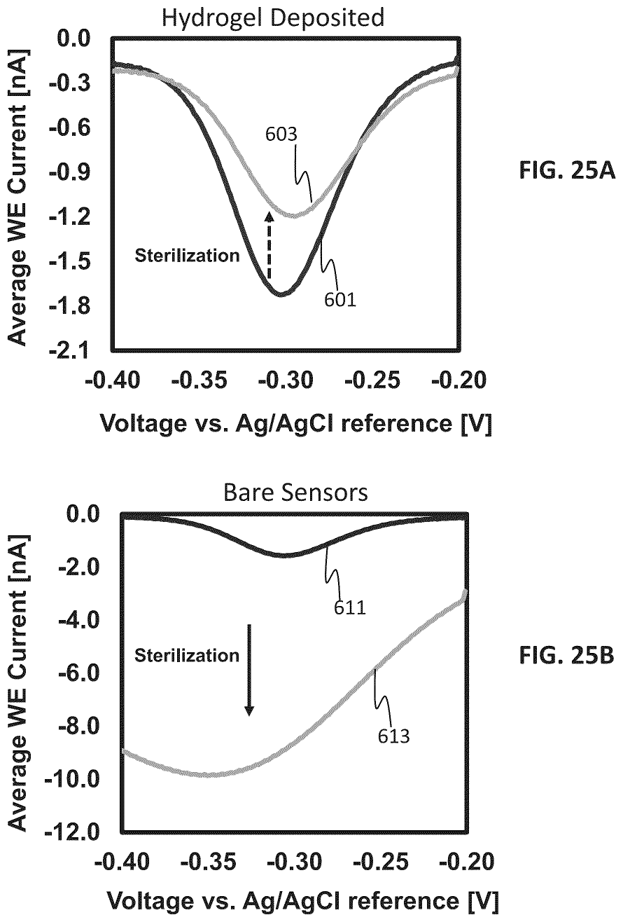

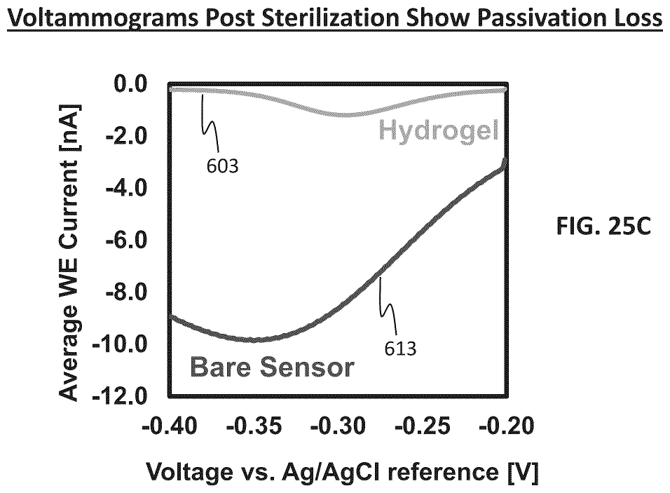

FIG. 25A shows a voltammogram showing an average (n=5) a SWV current in the presence of 1 μM cortisol measured with a hydrogel-coated working electrode 2110 before and after e-beam sterilization. During the signal measurement, the sensor array was immersed in PBS with 2% dimethyl-sulfoxide (DMSO) with 1 μM cortisol. The square wave frequency used to measure the peak SWV current was 50 Hz. Line 601 shows the SWV current generated by the hydrogel-coated working electrode before e-beam sterilization, and line 603 shows the SWV current generated by the hydrogel coated working electrode after e-beam sterilization. As can be seen in the figure, the e-beam sterilization caused the peak SWV current to be reduced by about 30%.

FIG. 25B shows a voltammogram showing an average (n=5) a SWV current in the presence of 1 μM cortisol measured with a bare working electrode 2110 before and after e-beam sterilization. By contrast, when a bare working electrode without hydrogel was e-beam sterilized under the same sterilization conditions, the biorecognition layer 2114 became highly degraded. As a result of the e-beam sterilization, as shown in FIG. 25B, the SWV current became abnormally strengthened across the range of voltages (x-axis) so that the peak current correlated to charge transfer due to aptamer-cortisol binding was no longer readily apparent (compare line 611 showing the SWV current generated by the bare working electrode before e-beam sterilization, with line 613 showing the SWV current after e-beam sterilization of the bare working electrode). The abnormally strengthened SWV current indicates preferential degradation or “stripping” of the passivation element (in this example 6-mercapto-1-hexanol) by the e-beam exposure. The ability of the hydrogel layer to prevent or mitigate passivation loss due to e-beam exposure can also be seen in FIG. 25C that overlays line 603 showing the SWV current generated by the hydrogel coated working electrode after e-beam sterilization and line 613 showing the SWV current after e-beam sterilization of the bare working electrode.

### Example 3—Effect of Zwitterionic Peptide Passivation on Aptamer-Based Sensor Sensitivity

The effect of using a different passivation element in aptamer-based cortisol sensors was studied. The control cortisol sensor was a planar gold array with electrodes functionalized with a cortisol-binding aptamer, as described in Example 1 with reference to FIGS. 21A and 21C, using 6-mercaptohexanol (MCH) as the passivation molecule 2129 tethered to the working electrode surface along with the cortisol binding aptamers 2125. The cortisol signals generated by the control cortisol sensor were compared against cortisol signals generated by a similar cortisol sensor that differed only in the passivation molecule 2129, which for that sensor was a zwitterionic peptide (“ZP”). The ZP used in this example was a synthetic peptide having an amino acid sequence of EKEKEPPC (SEQ ID NO: 2), with the C-terminal cysteine residue modified with an amide group (SEQ ID NO: 10).

The monolayer of passivation molecules 2129 self-assembled on the surface of the gold electrode material 2112 plays an important role in the function of the aptamer-based sensor. The monolayer passivates the gold electrode surface after immobilization of the aptamer probe. Without the passivating layer, exposed gold would participate in undesired charge transfer events that mask the cortisol-dependent signal from the redox reporters 2126 (e.g. methylene blue) bound to the aptamers 2125. However, since the charge transfer events between the redox reporters and the gold surface that generate the cortisol-based signal occur through the passivation molecule monolayer, it was important to ascertain that the positively charged lysine residues and the negatively charged glutamic acid residues did not interfere with signal generation.

For the control cortisol sensor, the gold electrode surface was functionalized with MCH at a concentration of 5 mM in 1×PBS. For the ZP cortisol sensor, the gold electrode surface was functionalized with the ZP at a concentration of 7 mM in 1×PBS. The ON/OFF frequencies for the ZP cortisol sensors were 200 Hz/5 Hz (ON frequency of 200 Hz and OFF frequency of 5 Hz). The ON/OFF frequencies for the control (MCH) cortisol sensors were 50 Hz/5 Hz (ON frequency of 50 Hz and OFF frequency of 5 Hz). Each of the sensors were exposed to seven different concentrations of cortisol, ranging from 10⁻¹⁰ M to 10⁻⁶ M. The sensitivity of respective sensors at each cortisol concentration was measure as a % gain. The % gain at each concentration was calculated relative to the gain of the same sensor at the lowest cortisol concentration of 10⁻¹⁰ M.

FIG. 27 shows a plot of the magnitude of the cortisol signal expressed as a percent gain (Y axis) against range of cortisol concentrations (X-axis) generated by the control cortisol sensor and the ZP cortisol sensor. The error bars represent one standard deviation for the last four voltammogram peaks before the next concentration step. The plot of the cortisol signal gain as measured by the control cortisol sensor is presented as data points connected by a dashed line 2703. The plot of the cortisol signal gain as measured by the ZP cortisol sensor is presented as data points connected by a solid line 2705.

FIG. 27 shows similar effects on signal gain between using MCH and the EKEKEPPC zwitterionic peptide. As such, the presence of the charged functional groups in the lysine and glutamic acid of the zwitterionic peptide does not appear to interfere with the charge transfer between the methylene blue redox reporter and the gold electrode material that is necessary for generation of the cortisol signal. The plot shown in FIG. 27 indicates that the use of the zwitterionic peptide as the passivation element results in a sensor that is less sensitive (has lower signal gain) at higher cortisol concentrations (above 0.2 μM), but is more sensitive (has higher signal gain compared to control) at lower cortisol concentrations (below 0.2 μM). By way of example, at a lower cortisol concentration of 0.1 μM, the % gain of the cortisol signal produced by the control cortisol sensor was 8%, while the % gain of the cortisol signal produced by the ZP cortisol sensor was 12%. By contrast, at a higher cortisol concentration of 1 μM, the % gain of the cortisol signal produced by the control cortisol sensor was 26% while the % gain of the cortisol signal produced by the ZP cortisol sensor was 20%.

### Example 4—Deposition of Multi-Component Passivation Elements

The effect of using a multi-component passivation element in aptamer-based sensors was studied. For these studies, a cortisol sensor with a planar gold array with electrodes functionalized with a cortisol-binding aptamer, as described in Example 1 with reference to FIGS. 21A and 21C, was used as a starting point. Unlike the aptamer-based sensor of FIG. 21A, which deployed a single component passivation element, the below aptamer-based sensors comprise multi-component passivation elements based on that which is shown in FIG. 17J.

For some of the below evaluations, cortisol signals from control sensors were compared against cortisol signals generated by cortisol sensors comprising varied compositions of single-component or multi-component passivation elements. The varied compositions of single-component or multi-component passivation elements of the below examples deploy 6-mercapto-1-hexanol (MCH), 2-((3-((6-mercaptohexyl)thio)-2-methylpropanoyl)oxy)ethyl (2-trimethylammonium)ethyl)phosphate (PC) or a combination thereof, at varying concentrations. MCH, though compatible with aptamer structural switching and electron transfer, provides, on its own, sub-optimal passivation. PC comprises a zwitterionic phosphorylcholine head group, which may have antifouling properties in vivo, and may allow for improvements in stability of aptamer-based sensors, but in isolation provides weakened electron transfer reduced sensor gains (i.e., % change in peak current in response to the target analyte). In other words, MCH improves sensor signal while PC extends sensor lifetime. Control sensors were fabricated with single thiol passivation elements comprising MCH. The examples below explore a multi-component passivation element comprising MCH and PC that manages the disadvantages each contributes while improving overall performance.

### Example 4a

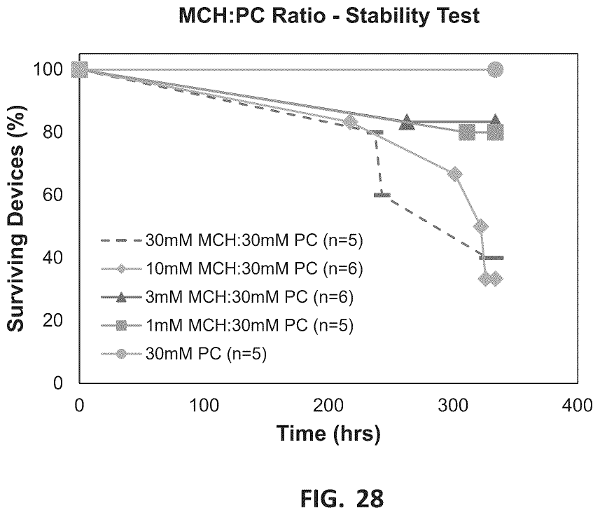

FIGS. 28-30 compare the stability of aptamer-based sensors, prepared in accordance with the methods of FIGS. 16J-18B, with different passivation element compositions, where the stability is determined by interrogating the sensors every 5 minutes at multiple SWV frequencies (5 Hz to 50 Hz) in PBS. Failure of the sensors was determined when current due to the oxygen reduction reaction passed a defined threshold, indicating passivation had dropped below an acceptable level. First, as shown in FIG. 28, the concentration of PC was held constant at 30 mM while the concentration of MCH was varied between 1 mM and 30 mM. Increasing the relative amount of MCH in the passivation element reduced stability in the sensors. In other words, increasing the relative amount of PC in the MCH and PC mixture increases sensor lifetime.

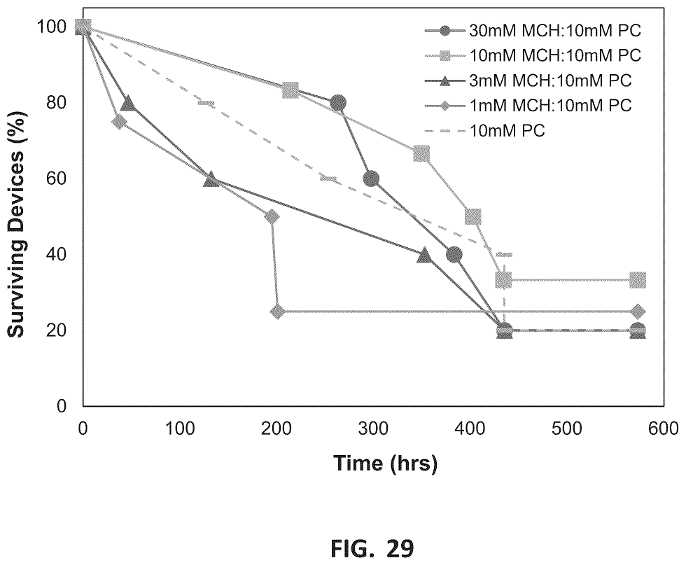

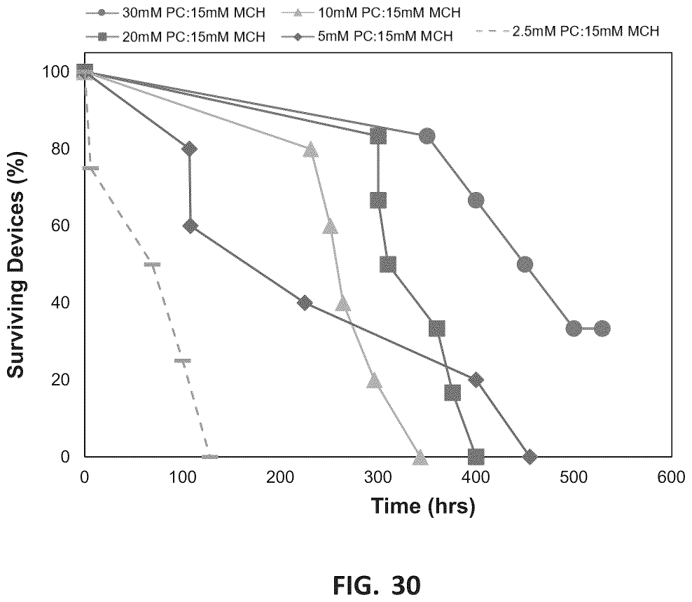

Next, as shown in FIG. 29, the concentration of PC was held constant at 10 mM while the concentration of MCH was varied between 1 mM and 30 mM. Increasing the relative amount of MCH in the passivation element increased stability in the sensors. Adding increasing amounts of MCH improved stability. Without being bound to any particular theory, this may be attributable to filling of defects that exists at relative low concentrations (10 mM) of a single-component PC-based passivation element. Next, as shown in FIG. 30, the concentration of MCH was held constant at 15 mM while the concentration of PC was varied. As depicted, there was a monotonic relationship with increasing concentration of PC and extended lifetime of sensors. In particular, using half-life, or the time at which half of the devices have failed, as a surrogate for lifetime, FIG. 30 shows the half-life of the devices as dependent upon relative PC concentration in the passivation element. As PC is increased from 2.5 mM to 5 mM to 10 mM to 20 mM to 30 mM, the resulting half-life increases from 69 hours to 167 hours to 258 hours to 310 hours to 450 hours, respectively.

### Example 4b

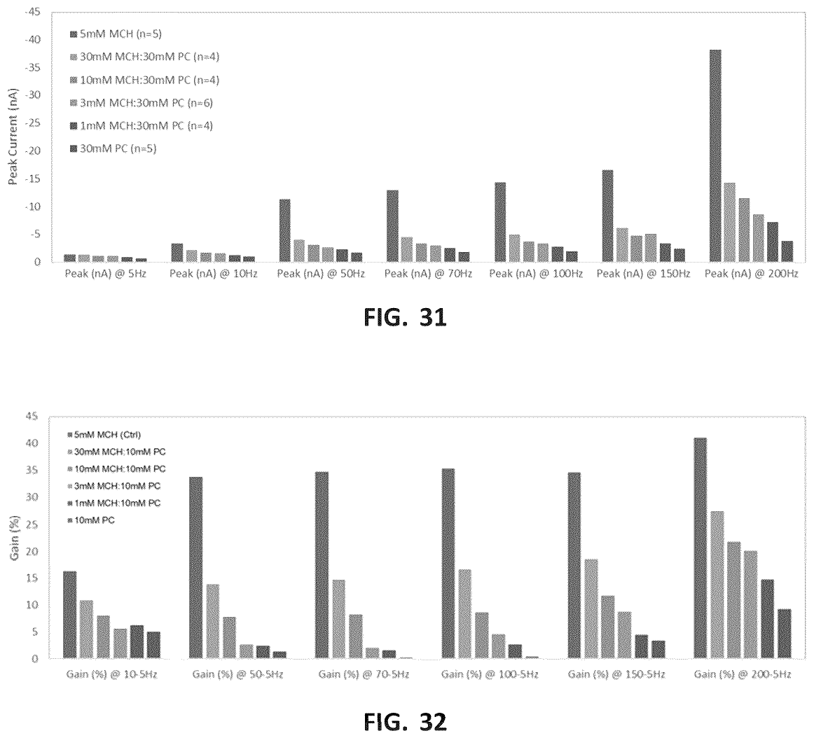

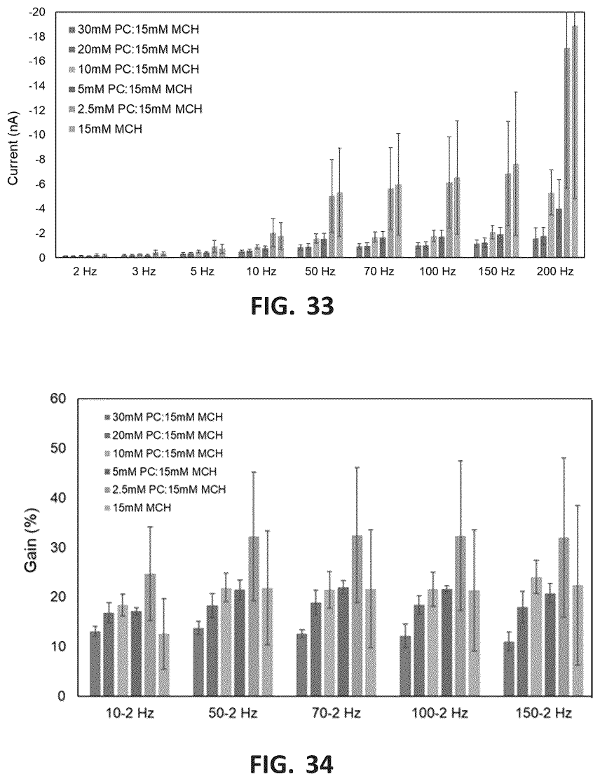

FIG. 31 and FIG. 33 compare peak currents for aptamer-based sensors having varying compositions of passivation element. Peak currents were measured by interrogating the sensors at multiple SWV frequencies (5 Hz up to 200 Hz). In FIG. 31, data is displayed in each group from left to right, with the far left being 5 mM MCH and the far right being 30 mM PC. In FIG. 33, data is displayed in each group from left to right, with the far left being 30 mM PC to 15 mM MCH and the far right being 15 mM MCH. The sensors were maintained in PBS in the absence of cortisol. The compositions of passivation element in FIG. 31 vary from single-component passivation elements (MCH only or PC only) to multi-component passivation elements (MCH and PC) while the compositions in FIG. 31 vary from MCH only to MCH and PC at varying ratios. As depicted in each of FIG. 31 and FIG. 33, peak currents increase as the ratio of MCH to PC increases. This can likely be attributed to MCH being a smaller molecule than PC and thus allowing electrons to be more easily tunnel to the gold electrode therethrough. Higher peak currents are beneficial to improve signal to noise (SNR) ratio and to reduce uncertainty in measurements.

### Example 4c

FIG. 32 and FIG. 34 compare kinetic differential gain measurements (i.e., positive gain frequency minus negative gain frequency) for aptamer-based sensors having varying compositions of passivation element. Gains were measured by interrogating the sensors at multiple SWV frequencies (10-5 Hz up to 200-5 Hz for FIGS. 32 and 10-2 Hz up to 150-2 Hz for FIG. 34). In FIG. 32, data is displayed in each group from left to right, with the far left being 5 mM MCH and the far right being 10 mM PC. In FIG. 34, data is displayed in each group from left to right, with the far left being 30 mM PC to 15 mM MCH and the far right being 15 mM MCH. FIG. 32 depicts sensor gain improving as the ratio of MCH to PC increases. FIG. 34 generally depicts the same trend, excepting pure MCH, where sensor gain improves as the ratio of MCH to PC increases.

### Example 4d

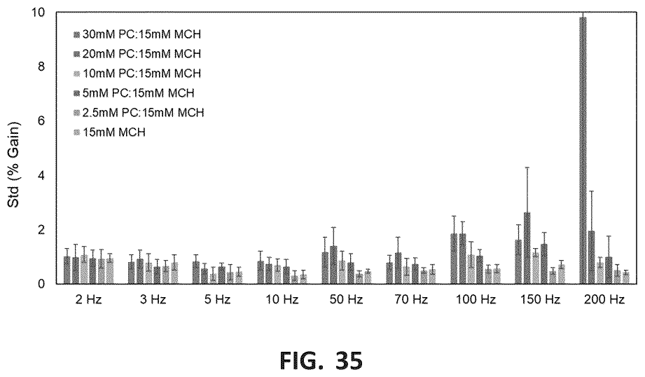

FIG. 35 compares standard deviations calculated across six replicate measurements of sensor gain at different frequencies (2 Hz up to 200 Hz). Measurements were obtained by interrogating aptamer-based sensors having varying compositions of passivation element. The sensors were interrogated at intervals one minute apart, thereby estimating noise level in the sensor gain. Data is displayed in each group from left to right, with the far left being 30 mM PC to 15 mM MCH and the far right being 15 mM MCH. Generally, as the ratio of MCH to PC increases, the noise level decreases. This can be appreciated in view of the results of Example 4a through Example 4c, where the sensors with higher percentages of MCH have higher peak currents and thus improved SNR (or lower noise levels), thus resulting in more stable gain calculations.

### Example 5—Exemplary Multi-Component Passivation Elements

A variety of multi-component passivation elements have been prepared. In each variation, deposition of the passivation element is preceded by deposition of a thiolated aptamer by exposing an electrode to a solution comprising the thiolated aptamer at 0.5 μM in PBS for between 12 hours and 48 hours.

In a first example, deposition of the multi-component passivation element includes exposing an electrode to a solution of 10 mM PC and 15 mM MCH in PBS.

In a second example, deposition of the multi-component passivation element includes exposing an electrode to a solution of 10 mM PC and 15 mM MCH with 2 mM methacrylate alkyl thiol ester in PBS. Methacrylate terminated alkyl thiol is copolymerized in situ with methacrylate-based monomers to form a protective membrane (aka biocompatible coating) over the sensor surface and simultaneously bond it to the surface.

In a third example, deposition of the multi-component passivation element includes exposing an electrode to a solution of 10 mM PC and 15 mM MCH with 5 mM thiolated polyethylene glycol in PBS. Addition of a PEG terminated thiol improves biocompatibility and macromolecular adsorption and biofouling.

In a fourth example, deposition of the multi-component passivation element includes exposing an electrode to a solution of 10 mM PC and 15 mM MCH with 1 mM 11-mercapto-undecanoic acid in PBS. In another example, 11-amino-1-undecanethiol was used in place of 11-mercapto-undecanoic acid. Addition of a carboxylic acid or primary amine terminated thiol provides an attachment point through carbodiimide/EDC/NHS type conjugation pathways for biocompatible promoting moieties, such as the peptide sequence RGD.

In a fifth example, deposition of the multi-component passivation element includes exposing an electrode to a solution of 10 mM PC and 15 mM MCH with 1 mM 11-mercapto-undecanoic acid conjugated to an RGD peptide in PBS. In another example, 11-amino-1-undecanethiol was used in place of 11-mercapto-undecanoic acid. In another example, a YIGSR peptide was used in place of an RGD peptide.

In a sixth example, deposition of the multi-component passivation element includes exposing an electrode to a solution of 10 mM PC and 15 mM MCH with 1 mM RGD peptide terminated thiol in PBS. Addition of a redox probe bound thiol provides an internal electrochemical potential indicator.

In a seventh example, deposition of the multi-component passivation element includes exposing an electrode to a solution of 10 mM PC and 15 mM MCH with 1 mM ferrocene terminated alkyl thiol in PBS.

In an eight example, deposition of the multi-component passivation element includes exposing an electrode to a solution of 10 mM 10-mercaptodecanesulfonate and 10 mM MCO in PBS. This combination combines a permanent anion thiol and permanent cation thiol in equal ratios, thereby creating a surface with equal positive and negative charges.

In a ninth example, deposition of the multi-component passivation element includes exposing an electrode to a solution of 5 mM 9-mercaptodecanesulfonate and 5 mM (9-mercaptononyl)-N,N,N-trimethylammonium bromide with 10 mM MCH in PBS.

### Example 6—Exemplary Hydrogel Additions

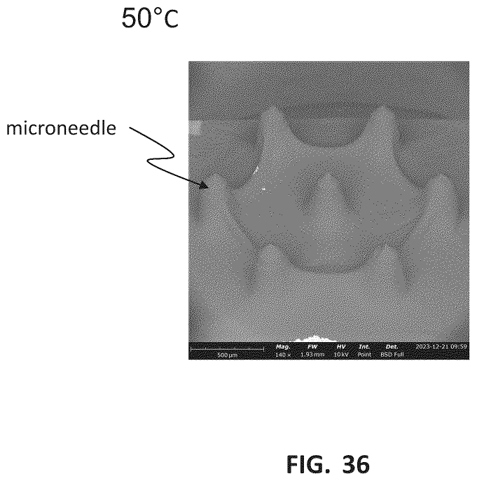

As described above in Example 1, the presence of the hydrogel biocompatible layer slowed the degradation of the function of the biorecognition layer that typically occurs when the working electrode is stored in air. FIG. 36 depicts a scanning electron micrograph of a sensor surface coated with a hydrogel biocompatible layer, described previously, and demonstrates hydrogel deposition coverage of microneedle bodies and respective electrodes.

As before, planar gold electrodes in a planar gold electrode array were utilized. Each planar gold electrode was an annular electrode on a distal portion of a microneedle body. The distal portion of the microneedle body was tapered. In optimizing hydrogel deposition and sensor coverage, temperature and hydrogel concentration were evaluated. FIG. 36 depicts an exemplary drop casted 2% agarose hydrogel at 50° C. The scale bar in the image is 500 μm. The hydrogel appears as darker gray in the images. Important for evaluating deposition coverage is the exposure of each sensor electrode surface and microneedle body as a result of a lack of coverage by the hydrogel. As can be observed upon visual inspection, drop casting of a 2% agarose hydrogel at 50° C. results in complete deposition coverage. This visual analysis was confirmed quantitatively by image processing, whereby the presence of sensor electrode surface would indicate a lack of coverage on a particular microneedle.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the inventions.

1. A sensor configured to generate a signal that is indicative of a concentration of an analyte in a fluid, the sensor comprising a working electrode comprising:
an electrode material;
a biorecognition layer disposed at least partially on the electrode material and comprising a biorecognition element that selectively and reversibly binds to an analyte; and
an at least partially desiccated hydrogel disposed on the biorecognition layer.

2. The sensor of claim 1, wherein the at least partially desiccated hydrogel has a moisture content of about 10%, about 20%, about 30%, about 40%, about 50%, about 60%, about 70%, about 80%, or about 90%.

3. The sensor of claim 1, wherein the at least partially desiccated hydrogel has a moisture content of about 0%.

4. The sensor of claim 1, wherein the hydrogel comprises one or a combination of two or more hydrophilic polymers selected from the group consisting of: agarose, poly(urethane), poly(N-vinylpyrrolidone), poly(acrylamide), poly(acrylic acid), poly(methacrylic acid), poly(2-hydroxyethyl methacrylate), poly(acrylic acid-co-acrylamide), poly(N-isopropyl acrylamide), poly(2-acrylamido-2-methylpropane sulfonic acid), poly(ethylene glycol), poly(vinyl alcohol), poly(lactic acid), poly(glycolic acid), poly(glycolic acid-co-lactic acid), collagen, alginate, hyaluronic acid, heparin, glycosaminoglycans, chitosan, Nafion, carboxymethylcellulose, and cellulose acetate.

5. The sensor of claim 1, wherein the hydrogel has a thickness of between about 0.001 μm and about 1000 μm, between about 0.01 μm and about 100 μm, between about 0.1 μm and about 10 μm, between about 0.05 μm and about 500 μm, between about 0.1 μm and about 200 μm, or between about 1 μm and about 1000 μm.

6. (canceled)

7. (canceled)

8. The sensor of claim 1, wherein the sensor further comprises a microneedle, and wherein the working electrode is coupled to the microneedle.

9. The sensor of claim 8, wherein the sensor further comprises a microneedle array, and wherein the microneedle is part of the microneedle array.

10. The sensor of claim 1, wherein the biorecognition element comprises an aptamer.

11. A method of manufacturing a sensor configured to generate a signal that is indicative of a concentration of an analyte in a fluid, the method comprising:
(a) providing a working electrode comprising an electrode material and a biorecognition layer disposed at least partially on the electrode material, wherein the biorecognition layer comprises a biorecognition element that selectively and reversibly binds to an analyte;
(b) applying a hydrogel on the biorecognition layer; and
(c) drying the hydrogel to an at least partially desiccated state.

12-64. (canceled)

65. A sensor configured to generate a signal that is indicative of a concentration of an analyte in a fluid, the sensor comprising a working electrode comprising:
an electrode material;
a biorecognition layer disposed at least partially on the electrode material and comprising a biorecognition element that selectively and reversibly binds to an analyte; and
a hydrogel disposed on the biorecognition layer, wherein
the biorecognition layer degrades at a rate of not more than about 3% per day when stored at an ambient temperature of between about 15 degrees Celsius and about 30 degrees Celsius, and an ambient humidity of between about 10% and about 80% relative humidity.

66-78. (canceled)

79. The sensor of claim 1, wherein the at least partially desiccated hydrogel comprises agarose.

80. The sensor of claim 79, wherein the agarose has an electroendosmosis of between about 0.09 and about 0.14.

81. The sensor of claim 79, wherein the agarose has a melting point of between about 75 degrees Celsius and about 97 degrees Celsius.

82. The sensor of claim 79, wherein the agarose has a sulfate content of less than about 0.20% w/w/.

83. The sensor of claim 1, wherein the biorecognition layer degrades at a rate of not more than about 3% per day when stored at an ambient temperature of between about 15 degrees Celsius and about 30 degrees Celsius, and an ambient humidity of between about 10% and about 80% relative humidity.

84. The sensor of claim 83, wherein the degradation of the biorecognition layer is measured based on a reduction of a signal produced by the electrode material when placed in a liquid comprising the analyte compared to a reference signal strength.

85. The sensor of claim 10, wherein the aptamer is functionalized with a redox reporter molecule.

86. The sensor of claim 1, wherein the biorecognition layer further comprises a passivation element disposed at least partially on the electrode material.

87. The sensor of claim 86, wherein the passivation element comprises one or more of thiol-based small molecules and zwitterions.

88. The sensor of claim 2, wherein the moisture content is a percentage of buffered saline in the at least partially desiccated hydrogel.
